(19) United States
(12) Patent Application Publication
(10) Pub. No.: US 2026/0369395 A1
(43) Pub. Date: Nov. 19, 2026

(54) MENTALESE: A FRAMEWORK TO TRAIN ARTIFICIAL INTELLIGENCE (AI) MODELS TO REASON IN A COMPACT STYLE

(71) Applicant: HealthGPT, Inc. DBA Hippocratic AI, Palo Alto, CA (US)

(72) Inventors: Kriti Aggarwal, Redmond, WA (US); Subhabrata MUKHERJEE, Palo Alto, CA (US); Munjal SHAH, Palo Alto, CA (US); Vishal PARIKH, Palo Alto, CA (US); Kumar Tanmay, Palo Alto, CA (US)

(73) Assignee: HealthGPT, Inc. DBA Hippocratic AI, Palo Alto, CA (US)

(21) Appl. No.: 19/742,388

(22) Filed: Jul. 15, 2026

Related U.S. Application Data
(63) Continuation-in-part of application No. 19/400,096, filed on Nov. 25, 2025.
Application No. 19/400,096 is a continuation-in-part of application No. 18/900,289, filed on Sep. 27, 2024.
Application No. 18/900,289 is a continuation of application No. 18/592,441, filed on Feb. 29, 2024, now Pat. No. 12,142,371.
(60) Provisional application No. 63/466,712, filed on May 15, 2023.
(60) Provisional application No. 63/611,762, filed on Dec. 18, 2023.
(60) Provisional application No. 63/961,878, filed on Jan. 16, 2026.

(51) Int. Cl. G16H 40/20; G10L 15/16; G10L 15/18; G10L 15/183; G10L 15/19; G10L 15/22; G10L 25/66

(52) U.S. Cl. CPC G16H 40/20; G10L 15/183; G10L 15/22; G10L 25/66; G10L 15/16; G10L 15/1822; G10L 15/19

(57) ABSTRACT

A computer-implemented method receives an input query and generates, by a machine learning model, a compressed reasoning trace comprising a sequence of symbolic reasoning steps. Individual symbolic reasoning steps comprise an operator token selected from a set of predefined reasoning operators and a symbolic expression token encoding a logical operation without natural language words. The compressed reasoning trace has a compressed token count that is less than a natural language token count of a natural language reasoning trace produced by a natural language model in response to the same input query, reducing inference-time computational load. The method identifies an answer operator in the compressed reasoning trace indicating that the compressed reasoning trace has concluded and extracts a final answer from an answer expression corresponding to the answer operator. A multi-stage training process comprising supervised fine-tuning followed by reinforcement learning trains the machine learning model to generate compressed reasoning traces.

### PRIORITY DATA & CROSS-REFERENCE TO RELATED APPLICATIONS

This application claims the benefit of U.S. Provisional Application No. 63/961,878, filed Jan. 16, 2026, and is a continuation-in-part of U.S. application Ser. No. 19/400,096, filed Nov. 25, 2025, which is a continuation-in-part of U.S. application Ser. No. 18/900,289, filed Sep. 27, 2024, now U.S. Pat. No. 12,142,371, issued Nov. 12, 2024, which is a continuation of U.S. application Ser. No. 18/592,441, filed Feb. 29, 2024, which claims the benefit of U.S. Provisional Application No. 63/611,762, filed Dec. 18, 2023, and U.S. Provisional Application No. 63/466,712, filed May 15, 2023. The disclosure of each of the foregoing applications is hereby incorporated by reference herein in its entirety.

### BACKGROUND OF THE TECHNOLOGY

### Field of the Technology

The present disclosure relates to machine learning (ML) model inference, and more particularly to systems and methods for generating compressed symbolic reasoning traces using a ML model trained to reason in a compact symbolic format, thereby reducing inference-time computational load while preserving reasoning accuracy. The present disclosure further relates to training methods for aligning ML models to generate such compressed symbolic reasoning traces.

### Description of the Related Art

Conventional LLM-based reasoning systems are not designed to generate reasoning outputs in a compact, token-efficient format at inference-time. Conventional LLM-based reasoning systems generate verbose natural language reasoning traces that restate the problem, enumerate intermediate steps in prose, and hedge before producing a final answer. This results in reasoning traces that are incompatible with real-time applications and resource-constrained deployment environments. Existing approaches to reducing reasoning trace length impose external token budgets that over-constrain difficult queries and under-constrain easy ones, rely on external length signals that do not capture the complexity of individual queries, or apply length-based penalties regardless of whether a long response is the only correct one in a training group.

### BRIEF DESCRIPTION OF THE DRAWINGS

A more complete understanding of the present disclosure may be obtained by reference to the following Detailed Description when taken in conjunction with the accompanying Drawings. In the figures, the left-most digit(s) of a reference number identifies the figure in which the reference number first appears. The same reference numbers in different figures indicate similar or identical items.

FIG. 1 is a block diagram of a system illustrating large language models (LLMs) to perform specialized healthcare-related functions, according to some implementations.

FIG. 2 is a block diagram illustrating an artificial intelligence (AI) architecture, according to some implementations.

FIG. 3 is a block diagram of a system that includes a low-latency conversational artificial intelligence (AI) architecture with a parallelized in-depth analysis and feedback loop, according to some implementations.

FIG. 4 is a block diagram of a system 400 illustrating a machine learning (ML) model generating a compressed reasoning trace based on an input query, according to some implementations.

FIG. 5 is a block diagram of a system 500 illustrating a structure of a sequence of symbolic reasoning steps in a compressed reasoning trace, according to some implementations.

FIG. 6 is a diagram of a system 600 illustrating a side-by-side comparison of a compressed reasoning trace generated by a machine learning (ML) model and a natural language reasoning trace generated by a natural language model in response to the same input query, according to some implementations.

FIG. 7 is a block diagram of a system 700 illustrating autoregressive generation of a sequence of symbolic reasoning steps by a machine learning model across successive time steps, according to some implementations.

FIG. 8 is a block diagram of a system 800 illustrating a reinforcement learning training process for a machine learning model using training prompt-response pairs, according to some implementations.

FIG. 9 is a block diagram of a system 900 illustrating a first training stage of a multi-stage training process in which a base model is trained by supervised fine-tuning to generate compressed reasoning traces, according to some implementations.

FIG. 10 is a block diagram of a system 1000 illustrating a second training stage of a multi-stage training process in which a fine-tuned machine learning model is further trained using reinforcement learning, according to some implementations.

FIG. 11 is a block diagram of a system 1100 illustrating a shorter length preference optimization (SLPO) reward structure for computing rewards of correct responses during reinforcement learning training of a machine learning model, according to some implementations.

FIG. 12 is a block diagram of a system 1200 illustrating a group relative policy optimization (GRPO) reward structure for computing normalized rewards during reinforcement learning training of a machine learning model, according to some implementations.

FIG. 13 is a table 1300 illustrating in-domain benchmark performance results of the machine learning model and baseline models across mathematical reasoning benchmarks, according to some implementations.

FIG. 14 is a table 1400 illustrating out-of-domain benchmark performance results of the machine learning model and baseline models across non-mathematical reasoning benchmarks, according to some implementations.

FIG. 15 is a chart 1500 illustrating a performance-efficiency frontier of the machine learning model and baseline models across compression rates and accuracy levels on mathematical reasoning benchmarks, according to some implementations.

FIG. 16 is a set of charts 1600 illustrating training dynamics of the multi-stage training process compared against direct reinforcement learning across response length, clip ratio, entropy loss, training time per step, and test performance metrics.

FIG. 17 is a flowchart of a method 1700 illustrating receiving an input query, generating a compressed reasoning trace, identifying an answer operator, and extracting a final answer, according to some implementations.

FIG. 18 is a block diagram of a system 1800 illustrating a machine learning model routing an input query to an LLM via application programming interfaces, databases, and agents to generate an output, according to some implementations.

FIG. 19 is a flowchart of a process 1900 illustrating training of a machine learning model configured to generate compressed reasoning traces, according to some implementations.

FIG. 20 illustrates an example configuration of a computing device that can be used to implement the systems and techniques described herein.

FIG. 21 is a diagram 2100 illustrating a comparison between responses generated by a large language model (LLM) when provided with a basic prompt and when provided with a more sophisticated, engineered prompt, according to some implementations.

FIG. 22 is a diagram 2200 illustrating a Retrieval-Augmented Generation (RAG) workflow, according to some implementations.

FIG. 23 is a diagram 2300 illustrating an overview of artificial intelligence (AI) agents, according to some implementations.

FIG. 24 is a block diagram of a system 2400 illustrating an inference hardware structure used to perform inferencing operations in an example artificial intelligence system, according to some implementations.

FIG. 25 is a block diagram of a system 2500 illustrating an edge-device inference system, according to some implementations.

FIG. 26 is a block diagram of a system 2600 illustrating a data center that can execute inference operations, according to some implementations.

FIG. 27 is a block diagram of a system 2700 illustrating a Model Context Protocol (MCP) architecture, according to some implementations.

FIG. 28 is a block diagram of a system 2800 illustrating a Model Context Protocol (MCP) workflow, according to some implementations.

FIG. 29 is a block diagram of a system 2900 illustrating an artificial intelligence system architecture that integrates multiple technological components to enhance language-model performance, according to some implementations.

### DETAILED DESCRIPTION

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed, or elements thereof can be implemented in the form of a computer product, including a non-transitory computer readable storage medium with computer usable program code for performing the method steps indicated. Furthermore, one or more implementations and clauses of the technology disclosed, or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

The systems and techniques described herein are designed to generate reasoning outputs in a compact, token-efficient format at inference-time, by training a large language model to generate reasoning outputs in a compact (“compressed”) symbolic format as a native output, without relying on post-processing, external length constraints, or translation from natural language, while maintaining the accuracy benefits that verbose natural language reasoning provides.

The systems and methods described herein train a machine learning (ML) model to generate a compressed reasoning trace in response to an input query. Thus, the verbose natural language chain-of-thought reasoning format used by conventional large language models (LLMs) is replaced with a compact symbolic reasoning format. The ML model trained by the disclosed systems and methods is (i) configured to reason through a sequence of symbolic reasoning steps rather than natural language text, with each step comprising an operator token and a symbolic expression token that together encode a complete reasoning step without natural language words, (ii) configured to produce a compressed reasoning trace whose token count is substantially less than the token count of a natural language reasoning trace produced by a natural language model on the same input query, thereby reducing inference-time computational load, (iii) trained in a first stage by supervised fine-tuning (SFT) that aligns the ML model to the symbolic reasoning format, and (iv) trained in a second stage by reinforcement learning (RL) that recovers the accuracy lost during the first stage while preserving the compact compressed reasoning trace format.

First, the ML model disclosed herein reasons through a sequence of symbolic reasoning steps rather than natural language text, which represents a fundamental departure from how conventional LLMs generate intermediate reasoning. Rather than producing sentences and paragraphs of natural language explanation, the ML model generates each reasoning step as an operator token followed by a symbolic expression token. The operator token identifies the type of reasoning operation being performed, such as variable assignment, equation definition, case branching, solving, or calculation. The symbolic expression token specifies the variables, numerical values, or function symbols on which the operation acts. Together, the operator token and the symbolic expression token encode a complete reasoning step without any natural language words. This structural absence of natural language words is the mechanism by which the compressed reasoning trace achieves a compressed token count that is substantially less than a natural language token count of a natural language reasoning trace produced by a natural language model on the same input query. The ML model identifies the answer operator in the compressed reasoning trace, and this identification triggers the ML model to terminate generation of the compressed reasoning trace. The final answer is extracted from the answer expression that corresponds to the answer operator.

Second, the reduced token count of the compressed reasoning trace translates directly into reduced inference-time computational load on the ML model. Generating fewer tokens requires fewer steps, less occupancy in memory, and less data movement across hardware at each inference call. The result is lower inference latency, lower memory consumption per query, and lower energy cost per response, while the ML model retains at least ninety percent of the accuracy of the natural language reasoning baseline on the same input query. In experimental evaluation, the ML model trained by the disclosed systems and methods generates compressed reasoning traces with four to sixteen times fewer tokens than a natural language reasoning baseline. Moreover, the ML model achieves up to five times lower inference latency as a direct consequence of the reduced token count. These computational gains make the ML model suitable for real-time and resource-constrained deployment contexts where natural language reasoning models cannot operate.

Third, the systems and methods train the ML model through a two-stage pipeline (e.g., reinforcement learning with verifiable rewards (RLVR)) that achieves symbolic format alignment and accuracy recovery in sequence. In the first stage, SFT trains the ML model on a dataset of training prompt-response pairs in which each response is a compressed reasoning trace in a symbolic reasoning language. This establishes the symbolic reasoning language as the native output format of the ML model. SFT alignment alone, however, causes a substantial drop in accuracy relative to the natural language reasoning baseline. This accuracy drop arises because the strict symbolic format enforces compressed reasoning traces that follow a single reasoning path, thereby reducing the exploratory branching tokens, such as self-correction and verification steps, that natural language chain-of-thought reasoning uses to improve accuracy at the cost of token count. It was not expected, and would not have been obvious, that a second stage of RL applied to the SFT-aligned model could substantially recover accuracy while maintaining the compact compressed reasoning trace format. The second stage of RL achieves this recovery by rewarding the ML model for generating shorter correct responses to training prompts while preserving the symbolic output format established by SFT.

Fourth, the disclosed second training stage of RL supports at least two reward formulations: Group Relative Policy Optimization (GRPO) and Shorter Length Preference Optimization (SLPO). Under GRPO, the ML model generates a group of candidate responses to each training prompt, and the reward of each individual response is computed as a normalizing reward based on the relative position of that response within a range defined by the least advantaged and the most advantaged responses in the group. SLPO extends this framework by introducing a conditional reward function that computes a length-preference reward for each correct response based on the relative position of its length within a range defined by the shortest and the longest correct responses in the group. A correct compressed reasoning trace that is shorter than other correct responses in the same group receives a higher reward. When only one correct response exists, or when all correct responses share the same length, SLPO assigns a uniform reward and does not penalize the correct response for its length. When no correct responses exist, SLPO assigns zero reward without distorting the reward signal. This conditional structure allows the ML model to generate shorter correct compressed reasoning traces without imposing a fixed token budget. Both GRPO and SLPO reward structures operate by producing scalar reward values that are used in a policy gradient computation to update the parameters of the ML model, producing a trained model whose parameter values encode a preference for generating shorter correct compressed reasoning traces, a measurable and verifiable change in the physical state of the ML model. Of course, it should be understood that GRPO and SLPO are merely examples of reward structures that can be used and other reward structures may be used in place of GRPO and SLPO.

The disclosed technology is directed to a specific improvement in computer technology rather than to an abstract idea. The technology claims a particular machine-implemented method in which an ML model generates a compressed reasoning trace composed of operator tokens and symbolic expression tokens, identifies an answer operator within the compressed reasoning trace, and extracts a final answer from the answer expression that corresponds to the answer operator. The answer operator performs a specific computational function: its identification by the ML model terminates generation of the compressed reasoning trace, preventing the model from generating additional tokens after a solution has been reached. This causal termination behavior is a concrete operational improvement to how the ML model functions during inference. The operator token and symbolic expression token together impose a structural constraint on the ML model output space that causes the model to generate a shorter token sequence than it would generate using natural language reasoning for the same input query. This structural constraint operates during token generation: at each generation step, the ML model selects the next token from an operator vocabulary or symbolic expression vocabulary that does not include natural language words. This structural constraint is how the ML model generates tokens, and how it reduces the number of generation steps required per inference call. This shorter token sequence reduces the inference-time computational load on the ML model as a direct consequence of the structural constraint, not as a result of an external budget or post-processing step. Thus, the disclosed technology results in an improvement to the computer itself because the compressed reasoning trace uses significantly (typically at least an order of magnitude less) memory compared to conventional systems.

As a first example, consider an artificial intelligence (AI) based virtual assistant engaged in a multi-turn healthcare conversation with a patient following a post-operative discharge. During the conversation, the patient reports shortness of breath and an elevated resting heart rate. The conversational AI receives this patient response as part of the ongoing conversation and passes a clinical input query to a second opinion module implemented as an ML model trained by the disclosed systems and methods. The second opinion module generates a compressed reasoning trace in response to the clinical input query. Each symbolic reasoning step in the compressed reasoning trace encodes a clinical reasoning operation as an operator token followed by a symbolic expression token, without any natural language words. For example, the second opinion module may encode a variable assignment step identifying the reported symptoms, a case branching step evaluating differential conditions, a calculation step assessing severity against clinical thresholds, and a terminal answer step indicating that the symptoms satisfy an escalation condition. Because the compressed reasoning trace contains substantially fewer tokens than a natural language reasoning trace produced by a natural language model on the same clinical input query, the second opinion module completes its analysis within the real-time latency window of the live conversation, allowing the conversational AI to act on the answer extracted from the answer expression without introducing a perceptible delay to the patient. The conversational AI then transfers the conversation to a medical professional based on the extracted final answer, consistent with the escalation condition identified in the compressed reasoning trace.

As a second example, an ML model trained by the disclosed systems and methods receives an input query comprising a multi-step mathematical problem. The ML model generates a compressed reasoning trace comprising a sequence of symbolic reasoning steps, each encoding a mathematical operation as an operator token followed by a symbolic expression token. For a problem requiring the ML model to determine the value of an unknown variable satisfying two simultaneous equations, the compressed reasoning trace may comprise, for example, the sequence of symbolic reasoning steps, as follows: a variable assignment step that names the unknown, an equation definition step that encodes the first constraint, a second equation definition step that encodes the second constraint, a solve step that applies algebraic reduction, a calculation step that evaluates the numerical result, and a terminal answer step from which the final answer is extracted. The entire compressed reasoning trace occupies a fraction of the tokens that a natural language reasoning model would generate for the same problem, which would include restating the problem in prose, narrating each algebraic manipulation in natural language, verifying the result by substitution, and hedging before committing to the answer. The ML model identifies the answer operator in the compressed reasoning trace, the identification of the answer operator triggers termination of generation of the compressed reasoning trace by the ML model and extracts the final answer from the answer expression that corresponds to the answer operator.

As a third example, an agentic AI system comprises multiple ML models that coordinate to complete a multi-step reasoning task. Individual ML models in the system are trained by the disclosed systems and methods to generate a compressed reasoning trace in response to an input query passed to it by an orchestrating agent. Because individual ML models generate a compressed reasoning trace comprising symbolic reasoning steps rather than natural language text, the token volume transmitted between ML models across the system is substantially less than the token volume that would be transmitted if individual ML models generated a natural language reasoning trace for the same input query. A first ML model receives an input query, generates a compressed reasoning trace, extracts a final answer from the answer expression that corresponds to the answer operator in the compressed reasoning trace, and passes that final answer as a new input query to a second ML model. The second ML model similarly generates a compressed reasoning trace and extracts a final answer. Because the inference-time computational load of each ML model is reduced in proportion to the reduction in token count of its compressed reasoning trace relative to a natural language reasoning trace, the agentic system completes multi-step coordinated reasoning tasks with lower cumulative latency, lower memory consumption, and lower energy cost per task than a system in which individual ML models reason in natural language.

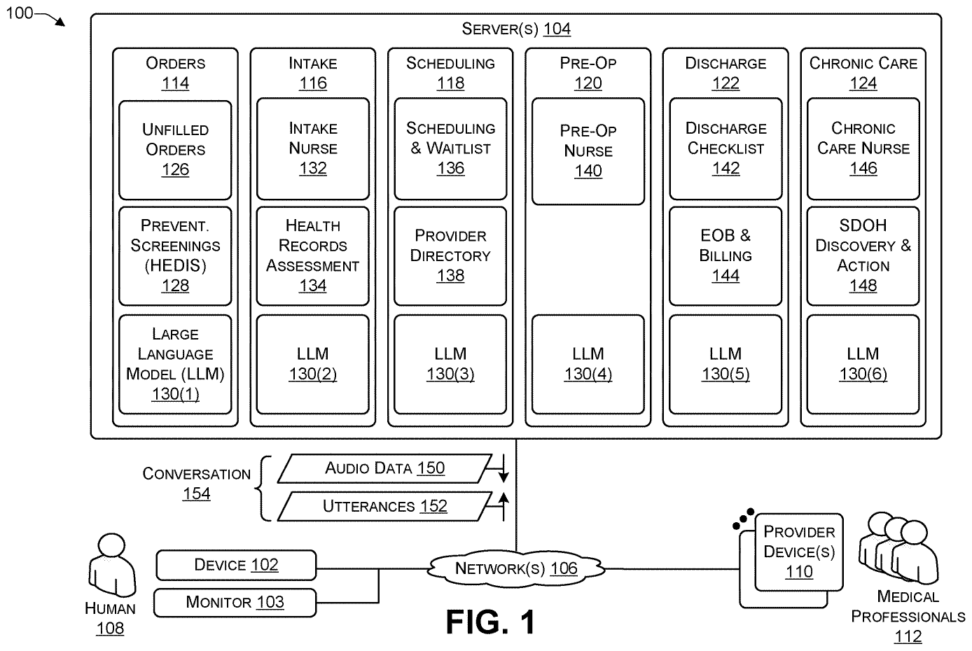

FIG. 1 is a block diagram of a system 100 illustrating large language models (LLMs) to perform specialized healthcare-related functions (e.g., roles), according to some implementations. The system 100 includes a device 102 connected to one or more servers 104 via one or more networks 106. The device 102 may be, for example, a smart phone, or another type of user device associated with a human 108. For example, the human 108 may be a current patient, a past patient, or a potential future patient. In some cases, the device 102 may be linked (“paired”) with a monitoring device (“monitor”) 103, such as a smart watch, a continuous glucose monitor (CGM), or another type of device that is capable of providing biometric readings associated with the human 108 to the device 102.

One or more provider devices 110 may be connected to the network 106. The provider devices 110 may be used by one or more medical professionals 112 (e.g., medical technicians, nurses, nurse practitioners, doctors, and the like) associated with a medical provider.

The server 104 may host multiple AI-based virtual assistants, such as the large language model (LLM) 130 virtual assistants. The virtual assistants LLM 130(1) to 130(6) shown in FIG. 1 each perform a particular healthcare-related role. Six LLMs 130 are shown purely for illustration purposes. It should be understood that the number of virtual assistants LLM 130 may vary depending on how the healthcare functions (roles) are divided. The division of the healthcare functions influences the algorithm design, training data, and other details associated with the virtual assistants 130. A different division of the healthcare functions may result in fewer or more than six virtual assistants LLM 130. For illustration purposes, the six functions (roles) include orders 114, intake 116, scheduling 118, pre-op 120, discharge 122, and chronic care 124.

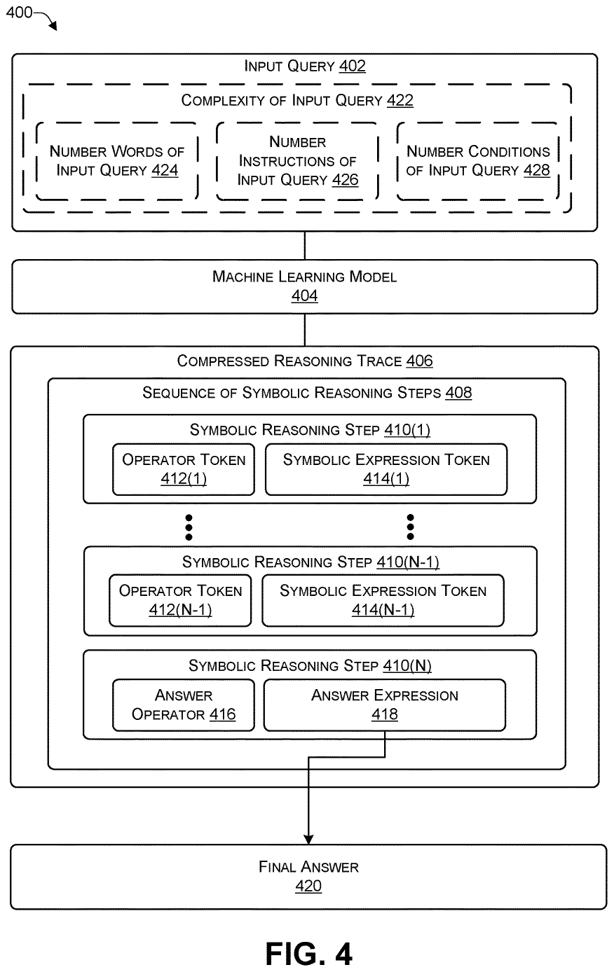

In some cases, one or more of the LLMs 130 is the machine learning model 404 of the present disclosure, as further described with reference to FIGS. 4 through 17. The machine learning model 404 generates the compressed reasoning trace 406, without natural language words, which reduces the number of tokens generated per inference relative to a natural language chain-of-thought model. This reduction in token count yields lower inference latency and lower memory consumption during inference. These are particularly meaningful advantages in a healthcare setting where multiple LLMs 130 may execute concurrently on the same server 104 and where response time directly affects patient experience.

The orders 114 role may include the LLM 130(1) engaging in a conversation 154 with the human 108 to resolve unfilled orders 126. Unfilled orders 126 refers to prescribed medications that the human 108 has not picked up, prescribed medical procedures or follow-up appointments that the human 108 has not scheduled, and so on. The orders 114 role may include the LLM 130(1) entering into a conversation 154 with the human 108 to perform preventative screenings 128, including gathering data relative to Healthcare Effectiveness Data and Information Set (HEDIS) calculations.

The conversation 154 may include audio data 150 output by one of the LLMs 130 and one or more utterances 152 from the human 108. The human 108 may speak the utterances 152 into a microphone of the device 102 and the device 102 may send the utterances 152 to the LLM 130 over the network 106. The LLM 130 may provide audio data 150 over the network 106 for playback by the device 102 to the human 108. The conversation 154 may be initiated by the LLM 130 or by the human 108.

The intake 116 role may include the LLM 130(2) engaging in the conversation 154 to perform the role of an intake nurse 132. For example, the conversation 154 may include the LLM 130(2) providing the human 108 with appointment-related information, such as a location and date and time associated with the appointment. The intake 116 role may include the LLM 130(2) engaging in the conversation 154 to perform a Health Records Assessment (HRA), determine a Risk Adjustment Factor (RAF), or the like.

The scheduling 118 role may include the LLM 130(3) engaging in the conversation 154 with the human 108 to perform scheduling and waitlisting related actions. For example, the LLM 130(3) may schedule the human 108 for an appointment and/or waitlist the human 108 for a consultation, surgery, or another type of appointment. The scheduling 118 role may include the LLM 130(3) engaging in the conversation 154 with the human 108 to access a provider directory to select a provider for the human 108. For example, if the human 108 has been asked (e.g., by a primary care physician) to have a consultation with a specialist, then the LLM 130(3) may identify a particular specialist in the provider directory 138 and schedule for the human 108 a consultation with the particular specialist.

The pre-op 120 role may include the LLM 130(4) engaging in the conversation 154 and taking on the role of a pre-op nurse 140 to go through a pre-op checklist with the human 108. For example, the pre-op checklist may include various pre-op do's and don'ts, such as what to eat, what not to eat (e.g., avoid solid foods at least 24 hours prior to a colonoscopy, avoid eating 12 hours prior to a blood glucose test, etc.), what medications to take, what medications to avoid taking (e.g., no beta blockers prior to a treadmill stress test), type of clothing to wear (e.g., loose clothing prior to a treadmill stress test), and so on.

The discharge 122 role may include the LLM 130(5) engaging in the conversation 154 to go over a discharge checklist 142. For example, the discharge checklist 142 may include describing what to do and what not do (e.g., don't perform certain action for a particular period of time after certain types of surgery), reviewing medications to take (e.g., take X every day, take Y as needed, and the like), highlighting warning signs (e.g., call medical provider immediately if the human 108 experiences particular symptoms, such as shortness of breath, dizziness, blurry vision, or the like), making follow-up appointments, and the like. The discharge 122 role may include the LLM 130(5) providing information regarding insurance coverage, explanation of benefits (EOB), and other billing-related information (e.g., amount that insurance should cover, amount that the human is responsible for paying, and so on).

The chronic care 124 role may include the LLM 130(6) performing the role of a chronic care nurse when engaging in the conversation 154 with the human 108. For example, the chronic care 124 role may include reviewing medical records and test results, and providing information regarding self-management, such as direct/nutrition suggestions, exercise suggestions, advice on managing prescription regimens, and information regarding monitoring symptoms. The chronic care 124 role may include the LLM 130(6) engaging in the conversation 154 to determine social determinants of health (SDOH), which are the non-medical factors that influence health outcomes. SDOH include the conditions in which people are born, grow, work, live, and age, and the systems shaping the conditions of daily life, such as economic policies, development agendas, social norms, social policies, and political systems.

Thus, a medical provider may deploy AI engines, in the form of an LLM or similar AI, to perform specialized health-related tasks. Each AI engine may be designed and trained for a particular purpose, such as screening, intake, scheduling, pre-op, discharge, chronic care, and the like. In some cases, one or more of the LLMs 130 is implemented as the machine learning model 404 of the present disclosure, the benefits include lower inference-time token count, reduced inference latency, and reduced memory consumption relative to a model that generates natural language chain-of-thought reasoning. The advantages of using AI engines include (i) reducing costs compared to using a human, (ii) freeing up humans to perform in-person functions, (iii) performing health-related tasks at times convenient to patients including outside regular business hours, and (iv) reducing human error when performing health-related tasks.

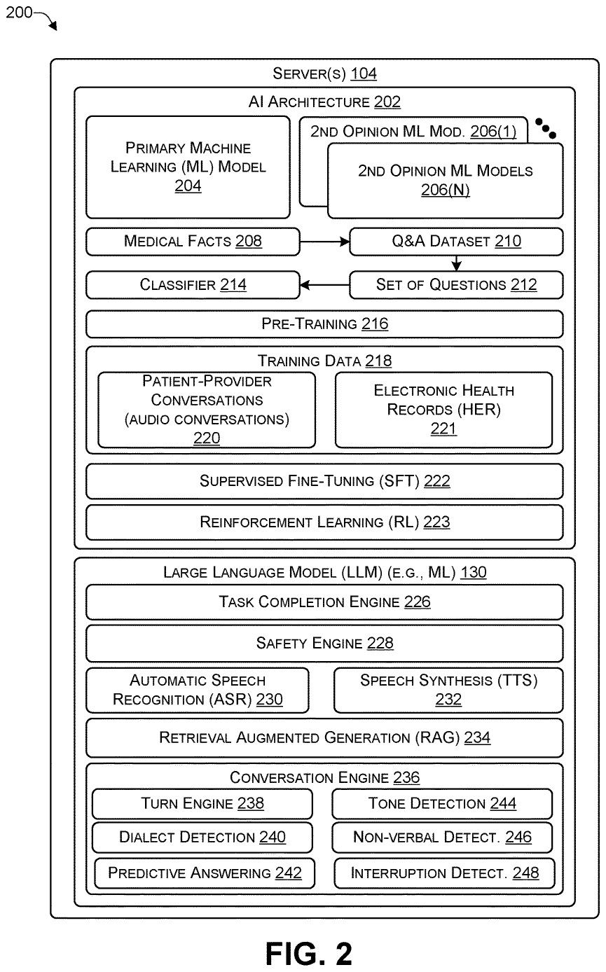

FIG. 2 is a block diagram 200 illustrating an AI architecture 202, according to some implementations. The AI architecture 202 may be used to implement one or more of the LLMs (e.g., LLM 130(1), 130(2), 130(3), 130(4), 130(5), and 130(6) of FIG. 1) described herein.

The AI architecture 202 may include a primary ML model 204 which implements a primary role, such as screening, intake, scheduling, pre-op, discharge, chronic care, or any combination thereof. One or more second opinion ML models 206(1) to 206(N) (N>0) may be associated with the primary ML model 204. The second opinion ML models 206 are AI models, such as LLMs, that are trained to perform a more in-depth analysis than the primary ML model 204. Based on information extracted from the conversation 154 of FIG. 1, one of the second opinion ML models 206 may perform a detailed analysis while the human 108 is engaged in the conversation 154 with one of the LLMs 130. For example, during a discharge call, the primary ML model 204 walks through the discharge checklist 142 while the second opinion ML model 206 concurrently reviews the patient's medication list against known contraindications. In a first case, where implemented using natural language reasoning, the primary ML model 204 operates at 50 ms latency and the second opinion ML model 206 operates at 200 ms latency, such that a contraindication warning may arrive after the primary ML model 204 has already advanced past the medication step of the checklist. In a second case, where both models are implemented as the machine learning model 404 of the present disclosure, the primary ML model 204 operates at approximately 10 ms and the second opinion ML model 206 operates at approximately 40 ms. This timing ensures the contraindication analysis completes before the primary ML model 204 advances past the medication step and allows the warning to reach the patient within the same conversational turn. If a second opinion ML model 206 determines that particular information provided by the LLM 130 during the conversation 154 can be clarified or expanded upon, the second opinion ML model 206 may provide, to the LLM 130, the particular information along with a suggestion regarding how to present the particular information to the human 108. In this way, the primary ML model 204 and the second opinion ML models 206 are part of a low-latency conversational AI architecture with a parallelized in-depth analysis feedback loop for healthcare-related applications. The purpose of reducing latency is to prevent the human 108 from hanging up in frustration or erroneously concluding that the conversation has concluded or the virtual assistant has encountered an error.

A large corpus of medical facts 208 are gathered and converted into a Question and Answer (Q&A) dataset 210. The Q&A dataset 210 may be used to create a set of questions 212. For example, medical facts 208, such as a description of an illness from a textbook, may be converted into a Q&A structure including “What is <illness>?”, “What are the symptoms of <illness>?”, “How is <illness> treated?”, and so on. Similarly, tables and other structured data included in the medical facts 208 are converted into a Q&A structure. One or more additional LLMs may be used to determine whether a particular question in the set of questions 212 is relevant. A classifier 214 (e.g., a support vector machine (SVM) or another type of classifier) is used to determine a distance between individual questions and their corresponding answers to determine if the answer is correct. In pre-training 216, a question is a prompt presented to the ML models 204, 206 and the answer is the response predicted by the ML models 204, 206. The Q&A dataset 210 may be used to fine tune the ML models 204, 206 via instruction tuning on prompt-response pairs in a supervised fashion. In some cases, the Q&A dataset 210 also serves as source material for generating training prompt-response pairs 802 in which each response is a compressed reasoning trace 406, as described in further detail with reference to FIG. 4. The AI architecture 202 uses the resulting training prompt-response pairs 802 to perform SFT 222 and/or RL 223 of the ML models 204, 206 on medical content. By training the ML models 204, 206 on medical content, the ML models 204, 206 are more suited to providing faster and clinically correct answers.

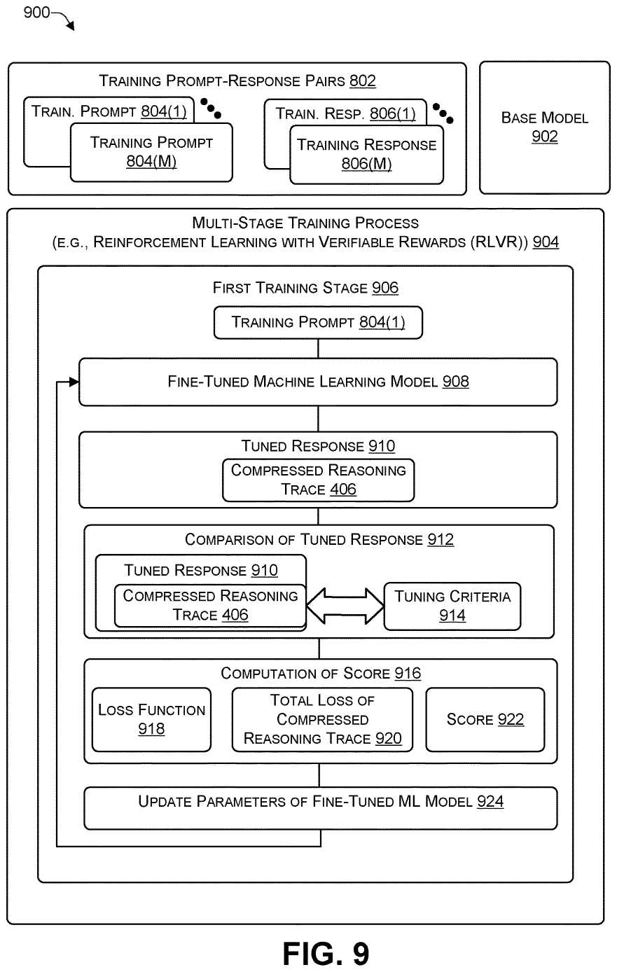

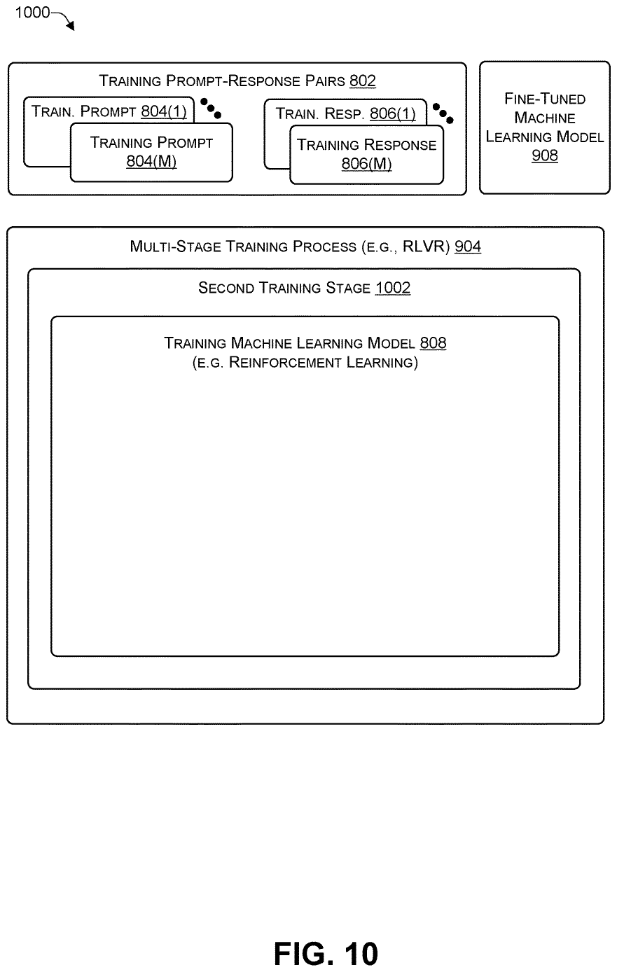

Training data 218 includes human-provider (e.g., patient-medical professional) conversations 220 in the form of audio data and electronic healthcare records (EHR) 221. The AI architecture 202 trains the ML model (e.g., LLM 130) using SFT 222, in which the ML model (e.g., LLM 130) is trained on prompt-response pairs in a supervised fashion. The AI architecture 202 further trains the ML model (e.g., LLM 130) using RL 223, in which the ML model (e.g., LLM 130) is trained using a reward function. In some cases, the reward function provides LLM 130 with a high reward for correct output and a low reward for incorrect output. In some cases, SFT 222 and RL 223 form the first and second stages, respectively, of a multi-stage training process (e.g., reinforcement learning with verifiable rewards (RLVR)) 904 described with reference to FIGS. 9 and 10. By training the ML model (e.g. LLM 130) using conversation data, the ML model (e.g., LLM 130) is more suited to engaging in the conversation 154 with the human 108 across the full range of healthcare roles described with reference to FIG. 1.

The LLM 130 (representing one of the LLMs 130(1) to 130(6) of FIG. 1) includes a task completion engine 226 that is tasked with completing a checklist during a particular conversation with the human. The task completion engine 226 enables the LLM 130 to complete tasks included in the checklist even while pursuing peripheral matters during the conversation, where such peripheral matters may help to build rapport between the virtual assistant and the human. For example, during the conversation with the human, the human may ask a question, such as a request for clarification of a particular topic (“Can I eat X prior to having the procedure?”). In response, the LLM 130 may provide information to answer the human's question. The LLM may also engage in banter about favorite foods, or the human's experiences with the foods, etc. After the LLM 130 answers the human's question and or engages in banter, the task completion engine 226 causes the LLM 130 to resume addressing items in the checklist. The LLM 130 may include a safety engine 228 to determine a medical accuracy of the information provided by the LLM 130 to the human. The safety engine 228 may determine when to perform an escalation and transfer a conversation between a human and an LLM to a medical professional.

The LLM 130 includes an automatic speech recognition module (ASR) 230 to perform speech recognition of the utterances 152 provided by the human 108. The LLM 130 includes a speech synthesis module 232 to perform text-to-speech (TTS) conversion. For example, after the LLM 130 looks up text-based information, the information is provided to the human in the form of speech using the speech synthesis module 232.

The LLM 130 includes a retrieval augmented generation (RAG) module 234. The RAG 234 is an AI framework to retrieve facts from an external knowledge base to provide the LLM 130 accurate and up-to-date information. The RAG 234 grounds the LLM 130 on external sources of knowledge to supplement the LLM 130. Implementing RAG 234 in an LLM-based question answering system enables the LLM 130 to access current, reliable facts, and the access to the sources of the LLM 130, enables output of the LLM 130 to be checked for accuracy. Using RAG, the AI-based virtual assistant described herein can provide accurate answers to a large number of questions that a human could not, such as menu options for a large number of local restaurants, details for a large number of insurance policies, details about a large number of hospital facilities (such as parking information, department locations, etc.).

The LLM 130 includes the conversation engine 236. The conversation engine 236 includes a turn engine 238, dialect detection 240, predictive answering 242, tone detection 244, nonverbal detection 246, and interruption detection 248. The turn engine 238 determines when the human has completed a turn and the LLM 130 has a turn in the conversation. The dialect detection 240 detects a dialect in the utterances of the human and causes the LLM 130 to output audio data having the same (or similar) dialect. For example, the dialect detection 240 may detect the dialect based on a particular word or a particular phrase in the human's utterances. To illustrate, the particular word or the particular phrase may be a vernacular term used in a particular geographic region.

The predictive answering 242 may predict multiple responses that the human may utter based on what the LLM 130 previously provided to the human. For example, the LLM 130 may provide a particular output (audio data) during the LLM's turn and then listen to the human during the human's turn. The predictive answering 242 may predict (i) possible human responses and (ii) corresponding answers based at least in part on the particular output provided during the LLM's turn. After the human has completed providing utterances in the human's turn, the LLM 130 may determine which of the predicted human responses the human provided and select a corresponding predicted answer. In some cases, where the LLM 130 is implemented as the ML model 404 of the present disclosure, each corresponding answer is generated as the compressed reasoning trace 406 requiring fewer tokens than if each corresponding answer is generated a natural language trace (e.g., natural language trace 604). In this way, LLM 130 (implemented as ML model 404) may pre-generate more possible human responses and their corresponding answers within the same compute and memory budget (than where LLM 130 is implemented as a natural language model 602), further reducing response latency after the human's turn.

The tone detection 244 is able to detect the tone and changes to the tone in the human's utterances during the conversation. For example, the tone detection 244 may be able to detect a tone of the human's utterances, correlate the tone with a particular mood, and adjust the audio data output by the LLM 130 accordingly. To illustrate, the tone of the human's utterances may be determined based on a pitch of the utterances, a volume of the utterances, a particular word in the utterances, a particular phrase in the utterances, or any combination thereof. If a particular tone is detected, such as that of anger or frustration, then the LLM 130 may adjust the audio data output accordingly, including changing the pitch of the audio data, changing a volume of the audio data, inserting a particular word or a particular phrase in the audio data, or any combination thereof. In some cases, if the tone of the human is regressing instead of progressing (e.g., the human has become more angry or more frustrated in a subsequent turn), then the LLM 130 may offer to transfer (“escalate”) the conversation to a human. The non-verbal detection 246 may detect the human coughing, sneezing, sighing, crying, or providing another nonverbal audio cue. Detecting one of the nonverbal audio cues may cause the LLM 130 ask additional (e.g., follow-up) questions, such as “How long have you had that cough?” (in response to detecting a cough), “How long have you had nasal congestion?” (in response to detecting a sneeze or sniffling), “Are you feeling okay?” (in response to detecting a loud sigh or crying), or the like. The interruption detection 248 may detect when the human is attempting to interrupt the LLM 130 and cause the LLM 130 to yield its turn and provide the human with a turn to enable the human to interject additional utterances into the conversation.

Thus, an AI architecture may include a primary model and one or more second opinion models to provide a low-latency conversational AI with a parallelized in-depth analysis and feedback loop in which the primary model engages in a low latency conversation with the human while the second opinion model gathers additional data and provides feedback (e.g., clarification or expansion of something that the primary model said to the human). Unlike conventional conversational AI's, the training data includes human provider audio-based conversations in addition to text-based training data. The LLM is trained using SFT and RL, in which a reward provides improved reasoning accuracy to the LLM. The LLM may use a task completion engine to avoid missing any items on a checklist of items to be covered with the human. The conversation engine used by the LLM may include a turn engine to determine when the human's turn has ended and the LLM's turn begins, dialect detection to understand and respond to the human in a local dialect, predictive answering to reduce latency by predicting possible answers while the human is talking, tone detection to detect emotional cues (e.g., based on a change in pitch, a change in volume, or the like) in the human's voice, non-verbal detection (e.g., coughing, sneezing, crying, or the like), and interruption detection to detect when the human is attempting to interrupt the LLM. In this way, the LLM is able to engage in a conversation with a human in a manner similar to a human. In some cases, in which the primary ML model 204 and the second opinion module 206 are each implemented as the ML model 404, the primary ML model 204 operates at approximately 10 ms latency and the second opinion module 206 operates at approximately 40 ms, compared to approximately 50 ms and 200 ms respectively under natural language reasoning. This allows the second opinion module 206 to complete its analysis and deliver feedback within the same conversational turn. The purpose of reducing latency is to prevent the human from hanging up in frustration or due to the human erroneously concluding that the conversation has concluded or the virtual assistant has encountered an error.

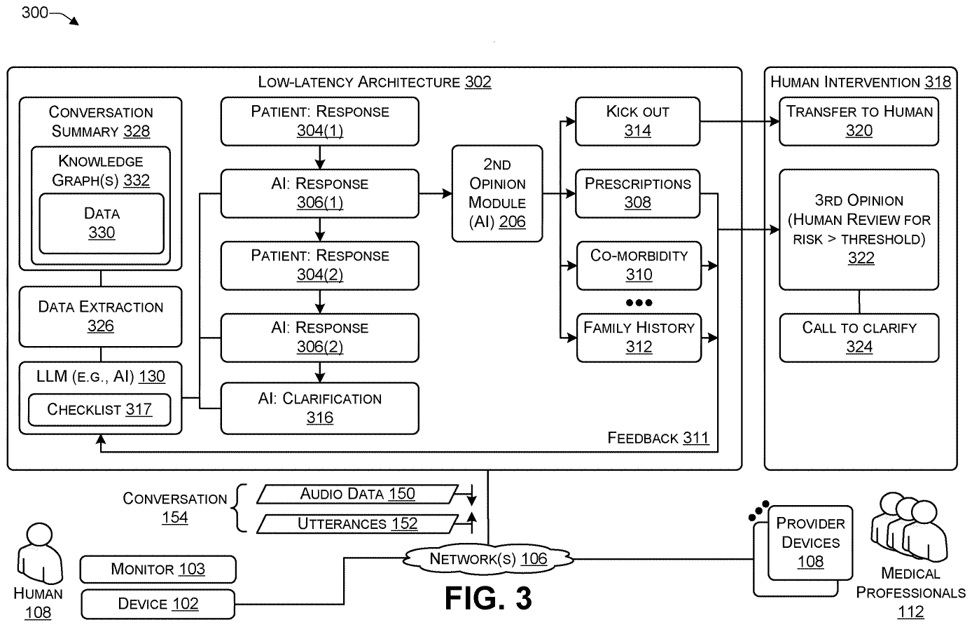

FIG. 3 is a block diagram of a system 300 that includes a low-latency conversational AI architecture with a parallelized in-depth analysis and feedback loop, according to some implementations. The system 300 includes a low latency architecture 302 in which the LLM 130 receives a (first) response 304(1) from the human 108. The LLM 130 may engage in the conversation 154 with the human 108 based on a checklist 317 that identifies tasks that the LLM 130 is to complete during the conversation 154. The LLM 130 provides a (first) response 306(1) to the human 108. The LLM 130 receives a (second) response 304(2) from the human 108 and provides a (second) response 306(2) to the human 108. While the LLM 130 is engaged in the conversation 154 with the human 108, the second opinion module 206 may, in parallel (substantially at the same time), do a lengthier “deep dive” by performing research into the human response 304(1) and the AI response 306(1). For example, the research may include prescriptions 308, co-morbidity 310, family history 312 and the like. The prescriptions 308 may include determining whether the human 108 is being prescribed prescriptions appropriate to the human's conditions, determining whether the human 108 is being prescribed the correct dosages of the prescriptions, determining whether the prescribed prescriptions have any undesirable interactions or contraindications, and other prescription related information. The prescriptions 308 may include a dosage engine that is invoked every time there is mention of a drug, dosage, or both. The co-morbidity 310 may include determining whether the human 108 has two or more medical conditions that may be related to an underlying cause or that have an undesirable interaction. The family history 312 may include determining whether the human's parents, siblings, or other relatives have a history of medical conditions similar to that of the human 108. In some cases, the LLM 130 and the second opinion module 206 are each implemented as the machine learning model 404 of the present disclosure, generating compressed reasoning traces 406 composed of sequences of symbolic reasoning steps 408 without natural language words, as further described with reference to FIGS. 4 through 17 and as discussed with reference to FIG. 2.

Based on determining the research into the prescriptions 308, the co-morbidity 310, and the family history 312, the second opinion module 206 may determine whether the AI response 306(1) was appropriate or whether the AI response 306(1) may be clarified. If the second opinion module 206 determines that the response 306(1) is appropriate, the second opinion module 206 does not take further action. If the second opinion module 206 determines that the response 306(1) may be clarified, then the second opinion module 206 may provide feedback 311 to the LLM 130, causing the LLM 130 to provide a clarification 316. For example, the human response 304(1) may be “Can I have soup?” and the AI response 306(1) may be “yes, you can have soup.”. The clarification 316 may be “I previously said you could have soup. I noticed that either you or at least one of your family members have high blood pressure, so please check the sodium content of any pre-packaged soups and avoid soups with high sodium.” In this example, “you . . . have high blood pressure” was determined by checking co-morbidity 310 and “at least one of your family members have high blood pressure” was determined by checking family history 312. As another example, the clarification 316 may be “I noticed that you are currently taking <1st prescription> in the morning. Instead of taking <2nd prescription> in the morning as I previously suggested, I suggest taking <2nd prescription> in the evening, around 12 hours after taking <1st prescription>.” In this example, by checking the prescription 308, the second opinion module 206 may determine that the patient is taking the 1st prescription and that the 2nd prescription (mentioned in the AI response 306(1)) is not to be taken at the same time as the 1st prescription and instruct the LLM 130 to clarify when to take the 2nd prescription. As a further example, the clarification 316 may be “I noticed that your potassium was high in your most recent blood work. While I had previously suggested eating more avocados (for the oil), I suggest not having more than one avocado a week to avoid raising your potassium further, as avocados are high in potassium”. In this example, by checking the co-morbidity 310 that includes test results (such as the results of a blood workup, e.g., comprehensive metabolic panel), the second opinion module 206 may determine that the patient has a high potassium level and that avocados are high in potassium and clarify how many avocados the human can have in a particular time frame.

In some cases, the second opinion module 206 may determine that providing the clarification 316 is insufficient and that the conversation 154 would benefit from human intervention 318. In such cases, the second opinion module 206 may use a kick out 314 engine (referred to herein as an escalation engine) to determine to transfer the conversation 154 to the medical professional 320 (e.g., human nurse or doctor). In addition, if the second opinion module 206 determines that a risk to the human 108 is greater than a predetermined threshold, then the second opinion module 206 may initiate a request for a third opinion 322 in which a human (e.g., a doctor, such as a specialist) reviews the human's case and takes appropriate action (e.g., provides a call (to the human 108) to clarify 324 the situation). The escalation engine will be invoked for qualitative statements that suggest symptoms that require further evaluation.

While the LLM 130 is engaged in the conversation 154 (e.g., the responses 304, 306), one or more background processes may perform data extraction 326 to extract data 330 (e.g., pertinent information, such as facts) to create a conversation summary 328 which is stored in the form of a knowledge graph 332 (or another type of data structure). The conversation summary 328 and the data 330 may enable the LLM 130 to quickly access earlier information in a lengthy conversation. With medical-related AI, conversations may be lengthy and involve many turns (e.g., 50 to 100 turns is common and the conversation may, in some cases, include 200 or even 300 turns). In such lengthy conversations, the human 108 may reference previously provided information (“referenced information”) in a current response. If the referenced information was provided early in the conversation, a conventional virtual assistant may have “forgotten” the referenced information and thus provide responses that are frustrating to the human. Alternatively, a conventional virtual assistant may have to scan through a transcript of the conversation to determine the referenced information. Such a scan may cause a lengthy delay and introduce latency into the conversation 154. By performing data extraction 326 and creating the conversation summary 328 and maintaining the knowledge graph 332 storing the data 330, the LLM 130 is able to quickly access the information (data 330) referenced by the human, thereby reducing latency. In some cases, where the LLM 130 is implemented as the machine learning model 404 of the present disclosure, the reduced token count of the compressed reasoning trace 406 further reduces per-turn inference latency independently of the knowledge graph 332 lookup. The purpose of reducing latency is to provide a natural sounding conversation and prevent the human from hanging up (i) in frustration or (ii) due to the human erroneously concluding that the conversation has concluded or the virtual assistant has encountered an error.

Thus, a low latency architecture for a conversational AI includes an AI, such as an LLM, engaged in a conversation with the human. While the AI is engaged in the conversation with the human, a second opinion module does a more in-depth analysis of information provided by the human. If the second opinion module determines that a response provided by the conversational AI is to be clarified, the second opinion module provides feedback that the conversational AI uses to provide a clarification to the human during the conversation. By parallelizing the second opinion module with the conversational AI, latency is reduced, thereby making the conversation more natural. In addition, while the conversational AI is engaged in the conversation with the human, a data extraction module (in parallel) extracts facts from each human response and creates a conversation summary in the form a data structure that can be quickly searched, such as a knowledge graph. The conversational AI can quickly access the conversation summary to look up information previously provided by the human that the human is referencing in a current turn in the conversation. In some cases, in which the primary ML model 204 and the second opinion module 206 are each implemented as the ML model 404, the primary ML model 204 operates at approximately 10 ms latency and the second opinion module 206 operates at approximately 40 ms, compared to approximately 50 ms and 200 ms, respectively, under natural language reasoning. This allows the second opinion module 206 to complete its analysis and deliver feedback within the same conversational turn. The access to the conversation summary enables the conversational AI to reduce latency because the conversation AI is not pausing the conversation to perform a search of a transcript of the conversation.

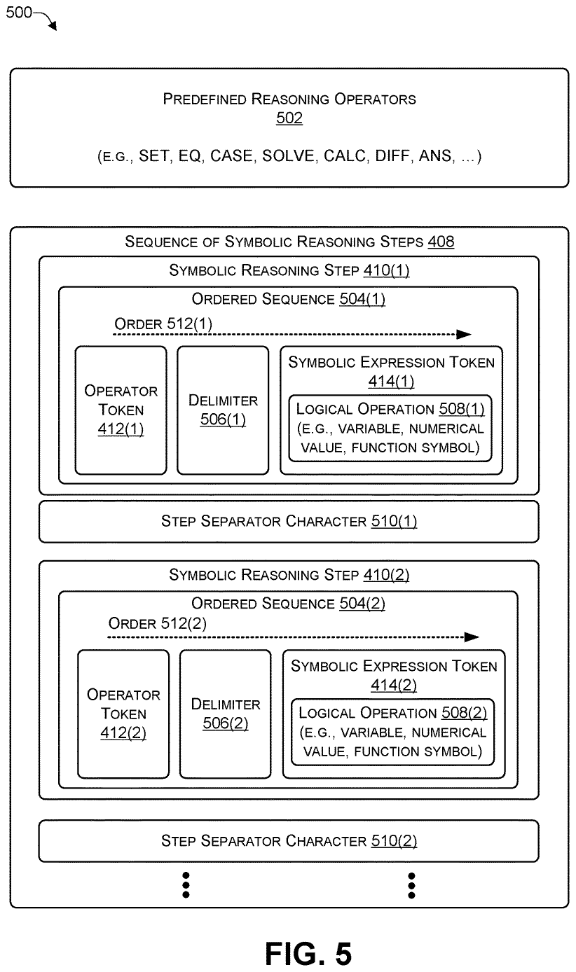

FIG. 4 is a block diagram of a system 400 illustrating how an ML model 404 receives an input query 402 and generates the compressed reasoning trace 406. In a conventional natural language chain-of-thought reasoning, a natural language model produces verbose natural language-based reasoning steps to arrive at a natural language final answer. In contrast, the ML model 404 generates the compressed reasoning trace 406 that is a sequence of symbolic reasoning steps 408 without natural language words. FIG. 4 is a description of the ML model 404 operating at inference time, and the elements shown here are referenced throughout the detailed description of FIGS. 5 through 17.

The ML model 404 receives the input query 402 and generates the compressed reasoning trace 406 based on the input query 402. The input query 402 may be a text-based or natural language question. The compressed reasoning trace 406 has a compressed token count that is less than a natural language token count of a natural language reasoning trace produced by the natural language model in response to the (same) input query 402. The lesser token count of the compressed reasoning trace 406 reduces an inference-time computational load on the ML model 404 relative to the natural language model. This reduction in inference-time computational load translates directly into lower inference latency and lower memory consumption during generation of the compressed reasoning trace 406.

The sequence of symbolic reasoning steps 408 comprises individual symbolic reasoning steps 410(1) through 410(N). Individual symbolic reasoning steps 410(1) through 410(N−1) comprise an operator token 412 and a symbolic expression token 414. The operator token 412(1) through 412(N−1) is selected from a set of predefined reasoning operators, as further described with reference to FIG. 5. Individual symbolic expression tokens 414 are paired with individual operator tokens 412 of the same individual symbolic reasoning steps 410. For example, symbolic expression token 414(1) is associated with operator token 412(1) in symbolic reasoning step 410(1), and so on through symbolic expression token 414(N−1) associated with operator token 412(N−1) in symbolic reasoning step 410(N−1). Individual symbolic expression tokens 414(1) through 414(N−1) encode a logical operation applicable to a variable, a numerical value, a function symbol, or any combination thereof. Because individual symbolic reasoning steps 410(1) through 410(N−1) contain no natural language words, the number of tokens required to represent individual symbolic reasoning steps 410 is less than the number of tokens required to represent the same reasoning step in natural language. This per-step token reduction contributes to the compressed token count of the compressed reasoning trace 406 being less than the natural language token count of the natural language reasoning trace.

The final symbolic reasoning step 410(N) in the sequence of symbolic reasoning steps 408 comprises the answer operator 416 as its operator token and an answer expression 418 as its symbolic expression token. The answer operator 416 is a specific operator selected from the set of predefined reasoning operators and indicates that the compressed reasoning trace 406 has concluded. The identification of the answer operator 416 by the ML model 404 riggers termination of generation of the compressed reasoning trace 406 by the ML model 404, preventing the ML model 404 from generating additional tokens after a solution has been reached. The answer expression 418 is associated with the answer operator 416 and encodes the value of the final answer 420. The system 400 identifies the answer operator 416 in the compressed reasoning trace 406 and extracts the final answer 420 from the answer expression 418 that corresponds to the answer operator 416 in the final symbolic reasoning step 410(N). The final answer 420 is the output of the ML model 404 for the input query 402. In some cases, the ML model 404 achieves an inference-time latency that is at least 25% less than the inference-time latency of the natural language model for the (same) input query 402. In some cases, the compressed token count of the compressed reasoning trace 406 is between 1% and 15% of the natural language token count of the natural language reasoning trace for the (same) input query 402. In some cases, the compressed reasoning trace 406 has at least 90% of the accuracy of the natural language reasoning trace generated by the natural language model in response to the (same) input query 402.

In some cases, the ML model 404 autoregressively generates the compressed reasoning trace 406 by sequentially predicting individual tokens in the compressed reasoning trace 406 conditioned on previously generated tokens. The sequence of symbolic reasoning steps 408 is ordered based on the time at which the ML model 404 generates individual symbolic reasoning steps 410. A symbolic reasoning step 410 generated at a later time is located after a symbolic reasoning step 410 generated at an earlier time in the sequence of symbolic reasoning steps 408. Successive individual symbolic reasoning steps 410 are concatenated using a step separator character to form the complete compressed reasoning trace 406, as further described with reference to FIG. 5.

In some cases, the ML model 404 generates the compressed reasoning trace 406 based further on a complexity 422 of the input query 402, as indicated by the dashed lines in FIG. 4. The complexity 422, the number of words 424, the number of instructions 426, and the number of conditions 428 are each shown in dashed lines to indicate that these are optional elements. The complexity 422 represents the number of reasoning operations that the ML model 404 requires to produce the final answer 420. The ML model 404 determines the complexity 422 based at least in part on a number of words 424 in the input query 402, a number of instructions 426 in the input query 402, a number of conditions 428 in the input query 402, or any combination thereof. A higher complexity 422 indicates that the ML model 404 requires more symbolic reasoning steps 410 to reach the final answer 420, and the ML model 404 scales the length of the compressed reasoning trace 406 accordingly.

Thus, the system 400 illustrates how the ML model 404 receives the input query 402 and generates the compressed reasoning trace 406 as a sequence of symbolic reasoning steps 408, each comprising an operator token 412 and a symbolic expression token 414 without natural language words, terminating at the answer operator 416, from which the final answer 420 is extracted. The compressed reasoning trace 406 has the compressed token count that is less than the natural language token count of a natural language reasoning trace for the same input query 402, reducing inference-time computational load, inference latency, and memory consumption. The ML model 404 therefore generates accurate reasoning outputs in a fraction of the token budget required by the natural language model in response to the (same) input query 402, while maintaining accuracy at or near the level of the natural language model.

FIG. 5 is a block diagram of a system 500 illustrating the internal structure of the sequence of symbolic reasoning steps 408 that the ML model 404 generates as the compressed reasoning trace 406. The system 500 describes the structural composition of individual symbolic reasoning steps 410 and how those steps are assembled into the compressed reasoning trace 406. FIG. 5 introduces several structural elements, including the predefined reasoning operators 502, the ordered sequence 504, the delimiter 506, the logical operation 508, and the step separator character 510, each of which is described in detail below.

The ML model 404 generates each operator token 412 by selecting from a set of predefined reasoning operators 502. The predefined reasoning operators 502 form a finite, closed vocabulary of symbolic operators that the ML model 404 uses to encode reasoning operations in the compressed reasoning trace 406. For example, the predefined reasoning operators 502 include a variable assignment operator (SET), an equation definition operator (EQ), a case branching operator (CASE), a solve operator (SOLVE), a calculation operator (CALC), a difference operator (DIFF), and an answer operator (ANS), among others. Individual reasoning operators in the predefined reasoning operators 502 correspond to a distinct class of logical operation that the ML model 404 performs during reasoning. At each generation step in which the ML model 404 generates the operator token 412, the ML model 404 selects from the predefined reasoning operators 502 rather than from an unconstrained natural language token distribution. This selection constraint reduces the output space of the ML model 404 at each operator-token generation step, which is a structural property of the ML model 404 operating under the symbolic reasoning language rather than a post-processing filter applied to the output. By constraining the operator token 412 of each symbolic reasoning step 410 to the predefined reasoning operators 502, the ML model 404 generates the compressed reasoning trace 406 that encodes reasoning in a compact, structured symbolic vocabulary rather than in unconstrained natural language text.

Turning to the internal structure of each symbolic reasoning step 410, the ML model 404 generates each symbolic reasoning step 410 as an ordered sequence 504. For example, the ordered sequence 504 of symbolic reasoning step 410(1) is labeled 504(1), and so on. Individual ordered sequences 504 comprise, in an order 512, the operator token 412, a delimiter 506, and the symbolic expression token 414. For example, the ordered sequence 504(1) includes, in the order 512(1), the operator token 412(1), the delimiter 506(1), and the symbolic expression token 414(1), within the ordered sequence 504(1) of the symbolic reasoning step 410(1), and so on. For example, the delimiter 506 of symbolic reasoning step 410(1) is labeled 506(1), and so on. Individual symbolic expression tokens 414 are paired with individual operator tokens 412 of individual symbolic reasoning steps 410. For example, symbolic expression token 414(1) is associated with operator token 412(1) in symbolic reasoning step 410(1), and so on. The ordered sequence 504 constrains the ML model 404 to generate the operator token 412 before the symbolic expression token 414 within each symbolic reasoning step 410, producing a consistent and parseable structure across all steps in the compressed reasoning trace 406.

The symbolic expression token 414 in each ordered sequence 504 encodes a logical operation 508. For example, the logical operation 508 of symbolic reasoning step 410(1) is labeled 508(1), and so on. Individual logical operations 508 are a variable, a numerical value, a function symbol, or any combination thereof. For example, operator token 412(1) may encode a variable assignment operator (SET) and the corresponding symbolic expression token 414(1) encoded by logical operation 508(1) may be “w”, representing the variable to which the assignment applies. Similarly, operator token 412(2) may encode an equation definition operator (EQ) and the corresponding symbolic expression token 414(2) may be “(180−5.5*w)=110” representing the specific equation to which the equation definition applies; and, the corresponding symbolic expression token 414(2) is encoded by individual logical operations 508(2), such as “(”, “1”, “8”, and so on. The logical operation 508 is associated with the operator token 412 of the same symbolic reasoning step 410, such that the operator token 412 identifies the reasoning operator and the symbolic expression token 414 encodes the specific operands and function symbols to which that operation applies. The absence of natural language words in the logical operation 508 significantly reduces the number of tokens required to represent each symbolic reasoning step 410 compared to a natural language representation of the same reasoning step.

The system 500 next assembles the sequence of symbolic reasoning steps 408 into the compressed reasoning trace 406 using the step separator character 510. The step separator character 510 of symbolic reasoning step 410(1) is labeled 510(1), and the step separator character 510 of symbolic reasoning step 410(2) is labeled 510(2). The ML model 404 concatenates successive individual symbolic reasoning steps 410 using the step separator character 510 to generate the compressed reasoning trace 406. Typically, the compressed reasoning trace 406 is a single linear reasoning path (in contrast to the natural language reasoning trace that is a multiple branched reasoning path). The step separator character 510 delimits the boundary between successive symbolic reasoning steps 410 in the sequence of symbolic reasoning steps 408, allowing the system 500 to parse the compressed reasoning trace 406 into its constituent symbolic reasoning steps 410 after generation.

In some cases, the sequence of symbolic reasoning steps 408 is ordered based on the time at which the ML model 404 generates individual symbolic reasoning steps 410. The symbolic reasoning step 410(2) generated at a later time is located after the symbolic reasoning step 410(1) generated at an earlier time in the sequence of symbolic reasoning steps 408. The ordering of symbolic reasoning steps 410 by generation time ensures that the compressed reasoning trace 406 captures the full logical progression of the ML model 404 from the input query 402 to the final answer 420.

The system 500 illustrates how the ML model 404 assembles the compressed reasoning trace 406 from the sequence of symbolic reasoning steps 408. The symbolic reasoning step 410 comprises the ordered sequence 504 that includes the operator token 412 selected from the predefined reasoning operators 502, the delimiter 506, and the symbolic expression token 414 encoding the logical operation 508 without natural language (words). The ML model 404 concatenates successive symbolic reasoning steps 410 using the step separator character 510 and orders them to form the compressed reasoning trace 406. The predefined reasoning operators 502 constrain the output space of the ML model 404 to a compact symbolic vocabulary. The ordered sequence 504 produces a consistent and parseable structure within each symbolic reasoning step 410. The step separator character 510 enables the system 500 to assemble and parse the compressed reasoning trace 406. Together, these structural elements enable the ML model 404 to generate the compressed reasoning trace 406 with the compressed token count that is less than the natural language token count of the natural language reasoning trace for the same input query 402.

The symbolic expression token 414 encodes each logical operation 508 as a variable, a numerical value, or a function symbol without natural language words, which substantially reduces the number of tokens required to represent each symbolic reasoning step 410 relative to a natural language representation of the same step. Because every symbolic reasoning step 410 in the compressed reasoning trace 406 applies this per-step token reduction, the cumulative effect across the sequence of symbolic reasoning steps 408 is a compressed token count 606 that is substantially less than the natural language token count of a natural language reasoning trace for the same input query 402. This reduction in token count reduces inference-time computational load, inference latency, and memory consumption on the ML model 404 relative to a natural language model resolving the same input query 402.

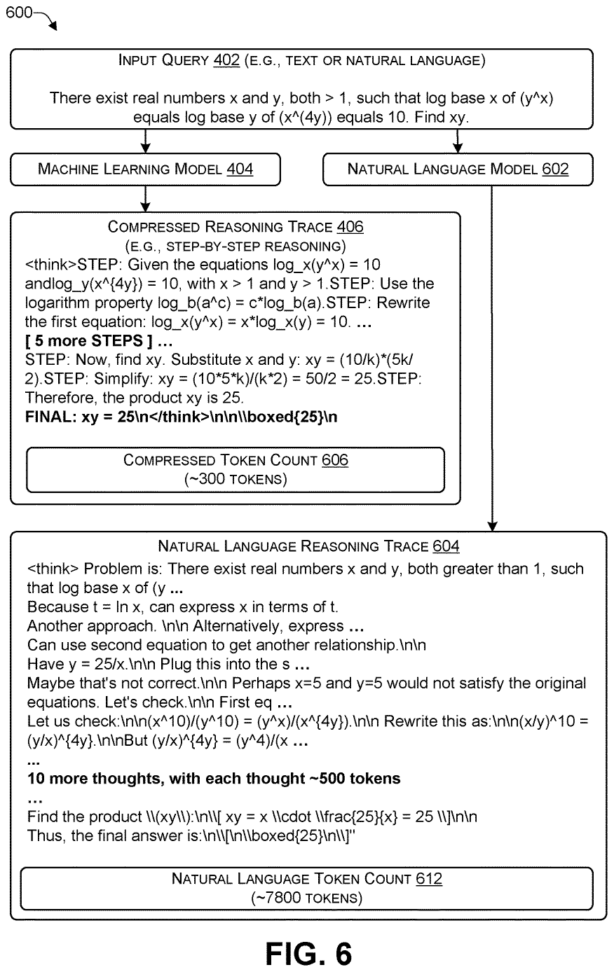

FIG. 6 is a diagram 600 illustrating an example of a side-by-side comparison of the compressed reasoning trace 406 generated by the ML model 404 against a natural language reasoning trace 604 generated by a natural language model 602, both in response to the same input query 402. Diagram 600 directly illustrates the token count reduction that the ML model 404 achieves relative to the natural language model 602.

The input query 402 in this example is a mathematical problem asking the reader to find the product xy, given that there exist real numbers x and y, both greater than 1, such that the logarithm base x of y to the power x equals the logarithm base y of x to the power 4y, and both equal 10.

The ML model 404 receives the input query 402 and generates the compressed reasoning trace 406 in response. The compressed reasoning trace 406 comprises a sequence of symbolic reasoning steps 408 without natural language. Typically, the compressed reasoning trace 406 is a single linear reasoning path, in contrast to the natural language reasoning trace 604 that typically comprises multiple branched reasoning paths. The system 600 identifies the answer operator 416 of “FINAL” in the compressed reasoning trace 406 and extracts the final answer 420, yielding the final answer 420 of 25 for the product xy. The compressed reasoning trace 406 has a compressed token count 606 of approximately 300 tokens.

In contrast, the natural language model 602 receives the same input query 402 and generates a natural language reasoning trace 604 in response. The natural language reasoning trace 604 comprises verbose natural language text, including repeated reformulations of the problem, exploratory reasoning paths, discarded intermediate steps, and extended prose explanations for each logical operation. For example, the natural language reasoning trace 604 includes phrases such as “Because t equals ln x, can express x in terms of t. Another approach. Alternatively, express . . . ” and “Maybe that's not correct. Perhaps x equals 5 and y equals 5 would not satisfy the original equations. Let's check . . . ” and approximately 10 further extended reasoning passages of similar length before arriving at the same final answer 420 of 25. The natural language reasoning trace 604 has a natural language token count 612 of approximately 7,800 tokens.

The comparison between the compressed token count 606 and the natural language token count 612 directly illustrates the inference-time computational load reduction that the ML model 404 achieves relative to the natural language model 602. The compressed token count 606 of approximately 300 tokens is approximately 3.8% of the natural language token count 612 of approximately 7,800 tokens, and thereby the compressed token count 606 within the range of 1% to 15% of the natural language token count 612. Moreover, the reduction in inference-time computational load translates into an inference-time latency of the ML model 404 that is at least 25% less than the inference-time latency of the natural language model 602 for the same input query 402. In some cases, the compressed reasoning trace 406 has at least 90% of the accuracy of the natural language reasoning trace 604 generated by the natural language model 602 in response to the same input query 402.

Thus, the system 600 illustrates how the ML model 404 resolves the input query 402 using the compressed reasoning trace 406 with the compressed token count 606 of approximately 300 tokens, compared to the natural language token count 612 of approximately 7,800 tokens required by the natural language model 602 for the same input query 402. This compression directly reduces the inference-time computational load on the ML model 404, lowering inference latency and memory consumption relative to the natural language model 602.

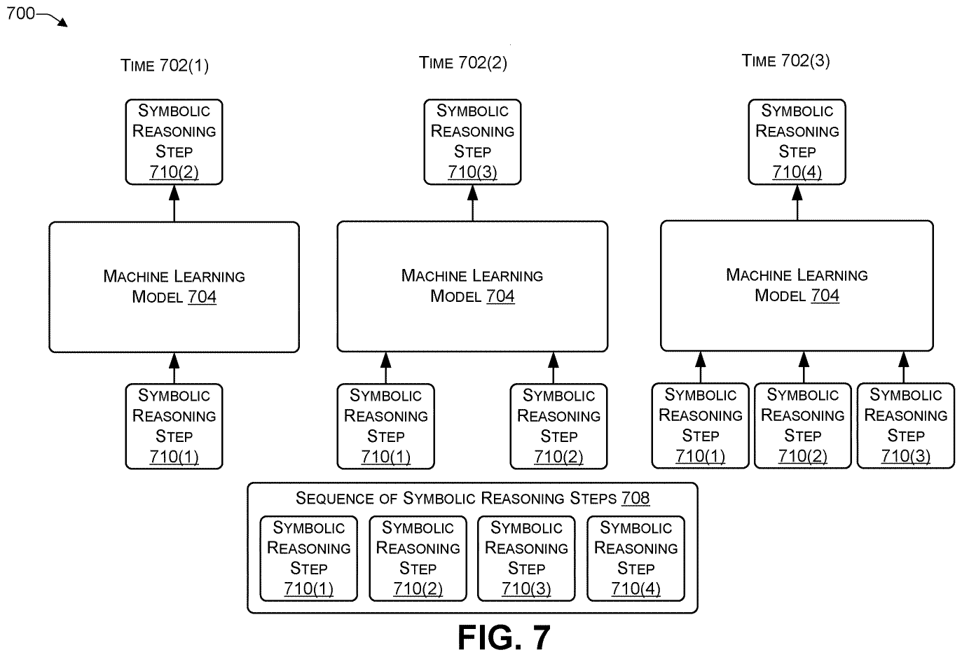

FIG. 7 is a block diagram of a system 700 illustrating that an ML model 704 autoregressively generates a sequence of symbolic reasoning steps 708 across successive time steps 702. The ML model 704 generates the sequence of symbolic reasoning steps 708 one step at a time, conditioned on previously generated steps. FIG. 7 shows three time snapshots, time 702(1), time 702(2), and time 702(3), each capturing the state of the sequence of symbolic reasoning steps 708 as the ML model 704 generates successive symbolic reasoning steps 710.

At time 702(1), the ML model 704 generates a symbolic reasoning step 710(2) in the sequence of symbolic reasoning steps 708. The ML model 704 sequentially predicts individual tokens in the symbolic reasoning step 710(2) conditioned on the input query 402 and previously generated tokens, including symbolic reasoning step 710(1). At time 702(1), the sequence of symbolic reasoning steps 708 contains symbolic reasoning steps 710(1), 710(2), representing an early state of the compressed reasoning trace 406 during generation.

At time 702(2), the ML model 704 generates a symbolic reasoning step 710(3) conditioned on the previously generated symbolic reasoning steps 710(1), 710(2), the input query 402, and any previously generated tokens. The ML model 704 appends symbolic reasoning step 710(3) to the sequence of symbolic reasoning steps 708 after symbolic reasoning steps 710(1), 710(2), using the step separator character 510 described with reference to FIG. 5. At time 702(2), the sequence of symbolic reasoning steps 708 contains symbolic reasoning steps 710(1), 710(2), and 710(3) in that order.

The sequence of symbolic reasoning steps 708 is ordered based on the time at which the ML model 704 generates individual symbolic reasoning steps 710. A symbolic reasoning step 710 generated at a later time is located after a symbolic reasoning step 710 generated at an earlier time in the sequence of symbolic reasoning steps 708. The ML model 704 continues generating successive symbolic reasoning steps 710 until it generates a symbolic reasoning step 710 comprising the answer operator 416. The answer operator 416 indicates that the compressed reasoning trace 406 has concluded.

Thus, the system 700 illustrates how the ML model 704 autoregressively generates the sequence of symbolic reasoning steps 708 one step at a time, appending each new symbolic reasoning step 710 in the order of generation and terminating upon producing the answer operator 416, yielding the compressed reasoning trace 406 that is compact and ordered. The compressed reasoning trace 406 contains only the symbolic reasoning steps 710 required to reach the final answer 420. This contributes to a compressed token count 606 that is between 1% and 15% of the natural language token count 612 of the natural language reasoning trace 604 and reduces inference-time computational load relative to the natural language model 602.

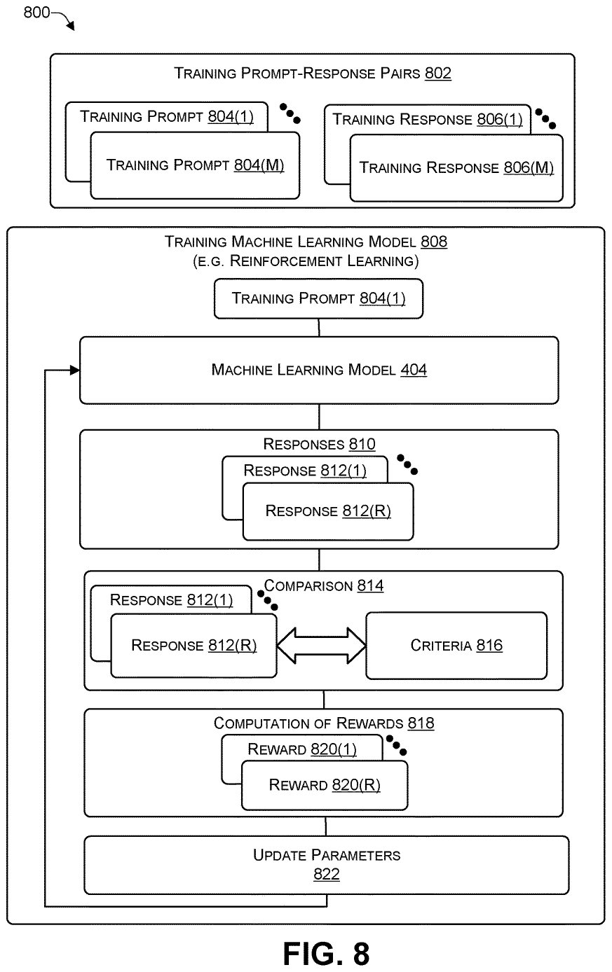

FIG. 8 is a block diagram of a system 800 illustrating how the ML model 404 is trained using RL 223. The system 800 determines training prompt-response pairs 802 for training ML model process 808. FIG. 8 describes the training process by which the ML model 404 learns to generate the compressed reasoning trace 406.

The training process begins with a set of training prompt-response pairs 802. The training prompt-response pairs 802 comprise individual training prompts 804(1) through 804(M) and their corresponding training responses 806(1) through 806(M). Individual training prompts 804 are input queries that the ML model 404 is trained to resolve. Individual training responses 806 (e.g., 806(1)) are ground truth responses corresponding to the training prompt 804 (e.g., 804(1)) of individual training prompt-response pairs 802. In some cases, the training responses 806(1) through 806(M) are compressed reasoning traces 406 comprising sequences of symbolic reasoning steps 408.

The training ML model process 808 uses RL 223 to train the ML model 404. For individual training prompts 804(1), the ML model 404 generates a set of responses 810 comprising individual responses 812(1) through 812(R). In some cases, the ML model 404 generates the set of responses 810 by autoregressively predicting individual tokens conditioned on previously generated tokens. The set of responses 810 captures a distribution of individual responses 812(1) through 812(R) that the system 800 subsequently evaluates.

Having generated the set of responses 810, the system 800 performs a comparison 814 of individual responses 812(1) through 812(R) against a set of criteria 816. The criteria 816 define the standard against which the system 800 evaluates individual responses 812. For example, the criteria 816 may include measuring the similarity between individual responses 812(1) through 812(R) and the training response 806(1) (ground truth) corresponding to the training prompt 804(1) that was the input. The comparison 814 produces a comparison signal for individual responses 812(1) through 812(R) in the set of responses 810.

Based on the comparison 814, the system 800 computes a reward 820 for individual responses 812(1) through 812(R), based at least in part on the comparison signal for individual responses 812(1) through 812(R). The computation of rewards 818 produces individual rewards 820(1) through 820(R) corresponding to individual responses 812(1) through 812(R). A higher reward 820 is assigned to a response 812 that satisfies the criteria 816, and a lower reward 820 is assigned to a response 812 that does not satisfy the criteria 816. The system 800 then updates the parameters 822 of the ML model 404 based on the rewards 820(1) through 820(R). The parameter update 822 biases the ML model 404 toward generating correct responses while preserving the accuracy of the correct responses. By iterating this process across the training prompt-response pairs 802, the ML model 404 learns to generate compressed reasoning traces 406 that reliably produce correct final answers 420 within the compact symbolic format of the sequence of symbolic reasoning steps 408.

Thus, the system 800 illustrates the RL 223 training process by which the ML model 404 learns to generate compressed reasoning traces 406 that reliably produce correct final answers 420 in the symbolic format of the sequence of symbolic reasoning steps 408. Iterating the training ML model process 808 across the training prompt-response pairs 802 and updating the parameters 822 based on rewards 820 trains the ML model 404 to generate compressed reasoning traces 406 with the compressed token count 606 that is between 1% and 15% of the natural language token count 612. This results in reducing inference-time computational load, inference latency, and memory consumption of the ML model 404 relative to the natural language model 602. FIG. 9 is a block diagram of a system 900 illustrating the first training stage 906 of the multi-stage training process (e.g., RLVR) 904. Whereas FIG. 8 describes the RL 223 training process in general terms, FIG. 9 describes the SFT 222 stage that precedes the RL 223 stage. The first training stage 906 trains a base model 902 on the training prompt-response pairs 802 to yield a fine-tuned ML model 908 that is configured to generate the compressed reasoning trace 406 in a symbolic reasoning language. The symbolic reasoning language is the native output format of the fine-tuned ML model 908 after completion of the first training stage 906.

The first training stage 906 begins with the base model 902 and the training prompt-response pairs 802. The base model 902 is a pre-trained ML model with native output format of natural language reasoning. The system 900 subjects the base model 902 to SFT 222 during the first training stage 906. The training prompt-response pairs 802 comprise individual training prompts 804(1) through 804(M) and their corresponding training responses 806(1) through 806(M). In some cases, the training responses 806(1) through 806(M) are compressed reasoning traces 406. The training prompt-response pairs 802 provide the base model 902 with paired examples of input queries and their corresponding compressed reasoning trace outputs, allowing the base model 902 to learn the symbolic reasoning language as its native output format.

During the first training stage 906, the base model 902 receives individual training prompts 804 (e.g., 804(1)) from the training prompt-response pairs 802 and generates a tuned response 910 to individual training prompts 804 (e.g., 804(1)). The tuned response 910 includes the compressed reasoning trace 406 that the base model 902 generates for the training prompt 804(1) during the current step of the first training stage 906. The system 900 performs a comparison of tuned response 912 by comparing the tuned response 910 against the corresponding training response 806(1). The comparison of tuned response 912 evaluates how closely the compressed reasoning trace 406 of the tuned response 910 matches the compressed reasoning trace 406 of the corresponding training response 806(1) in terms of symbolic structure, operator token selection, and symbolic expression token content.

Based on the comparison of tuned response 912, the system 900 applies tuning criteria 914 to compute a score 922 for the tuned response 910. The tuning criteria 914 define the standard against which the system 900 measures the quality of the tuned response 910. The system 900 performs a computation of score 916 that produces the score 922 reflecting how well the tuned response 910 satisfies the tuning criteria 914. The system 900 then applies a loss function 918 (e.g., entropy loss) to the score 922 to compute a total loss 920 of the compressed reasoning trace. The total loss 920 of the compressed reasoning trace quantifies the deviation of the tuned response 910 from the corresponding training response 806(1) in the training prompt-response pair 802. A lower total loss 920 of the compressed reasoning trace indicates that the compressed reasoning trace 406 of the tuned response 910 more closely matches the compressed reasoning trace 406 of the corresponding training response 806(1).

Based on the total loss 920 of the compressed reasoning trace, the system 900 updates the parameters of the fine-tuned ML model 908 by performing an update parameters of fine-tuned ML model 924 step. The update parameters of fine-tuned ML model 924 step adjusts the parameters of the base model 902 to reduce the total loss 920 of the compressed reasoning trace across the training prompt-response pairs 802. By iterating the first training stage 906 across individual training prompt-response pairs 802, the system 900 trains the base model 902 to generate tuned responses 910 that progressively more closely match the compressed reasoning traces 406 of individual training responses 806(1) through 806(M). The output of the first training stage 906 is the fine-tuned ML model 908, which is configured to generate the compressed reasoning trace 406 in the symbolic reasoning language as its native output format. The fine-tuned ML model 908 serves as the starting point for the second training stage 1002 described with reference to FIG. 10.

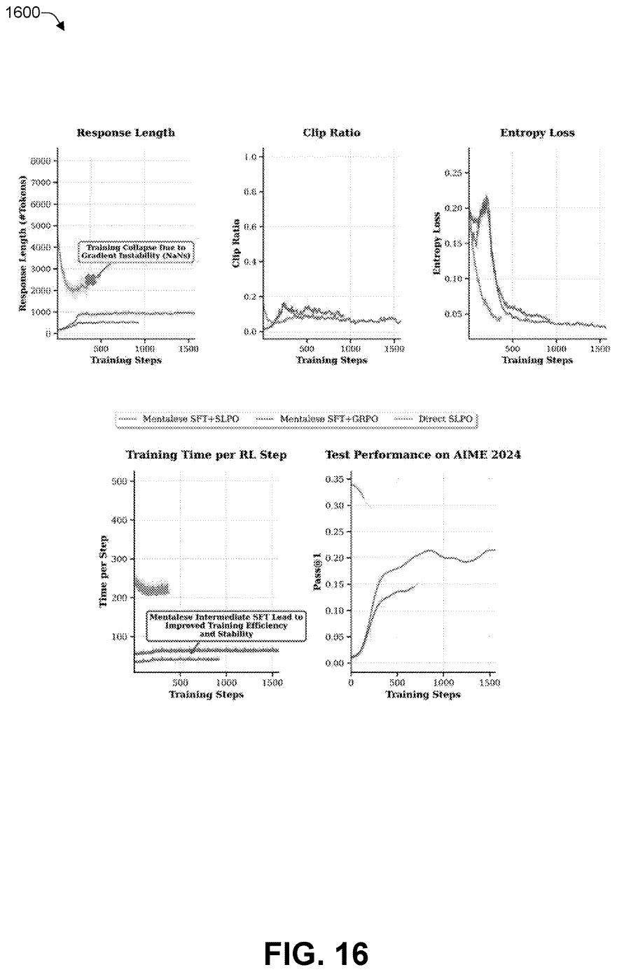

A key outcome of the first training stage 906 is that the fine-tuned ML model 908 generates the compressed reasoning trace 406 in the symbolic reasoning language as a native output format rather than as a post-processing transformation of natural language output. Prior to the first training stage 906, the base model 902 generates responses in natural language. After the first training stage 906, the fine-tuned ML model 908 generates responses directly in the symbolic reasoning language of the compressed reasoning trace 406. This native symbolic output format enables the RL 223 training stage of FIG. 10 to achieve stable and efficient reinforcement learning, as further described with reference to FIG. 16.

Thus, the first training stage 906 trains the base model 902 on training prompt-response pairs 802 in which individual training responses 806 are compressed reasoning traces 406, thereby producing the fine-tuned ML model 908 that generates the symbolic reasoning language as its native output format. The first training stage 906 reduces the token count of individual responses by approximately 7 to 10 times relative to the natural language reasoning traces 604 of the base model 902. This reduces the computational cost of each RL 223 training step in the second training stage 1002 and allows stable and efficient RL 223 training.

FIG. 10 is a block diagram of a system 1000 illustrating the second training stage 1002 of the multi-stage training process (e.g., RLVR) 904. FIG. 10 describes the second training stage 1002 in which the fine-tuned ML model 908 is further trained using RL 223. Together, the first training stage 906 and the second training stage 1002, in that order, form the multi-stage training process 904 that produces the ML model 404 configured to generate accurate and compact compressed reasoning traces 406 at inference time.

The second training stage 1002 takes two inputs: the fine-tuned ML model 908 produced by the first training stage 906 and the training prompt-response pairs 802. The training prompt-response pairs 802 comprise individual training prompts 804(1) through 804(M) and their corresponding training responses 806(1) through 806(M). The fine-tuned ML model 908 enters the second training stage 1002.

The second training stage 1002 applies the training ML model process 808 using RL 223 to the fine-tuned ML model 908, as described with reference to FIG. 8. The fine-tuned ML model 908 generates the set of responses 810 for individual training prompts 804, the system 1000 performs the comparison 814 against the criteria 816, computes rewards 820(1) through 820(R), and updates the parameters 822 to bias the fine-tuned ML model 908 toward generating correct and compact compressed reasoning traces 406.

The first training stage 906 plays a critical role in allowing the second training stage 1002 to succeed. Applying RL 223 directly to the base model 902 causes training instability due to gradient instability, as further described with reference to FIG. 16. The SFT 222 alignment of the first training stage 906 provides the second training stage 1002 with a fine-tuned ML model 908 that generates structurally consistent symbolic outputs, producing more stable gradient updates during RL 223 training, and allowing the fine-tuned ML model 908 to learn to generate shorter and more accurate compressed reasoning traces 406.

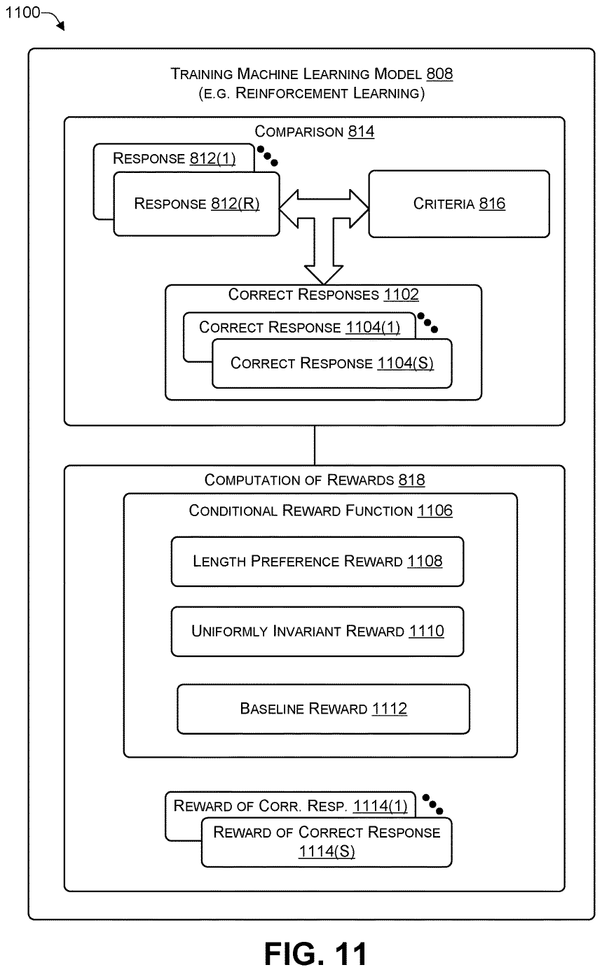

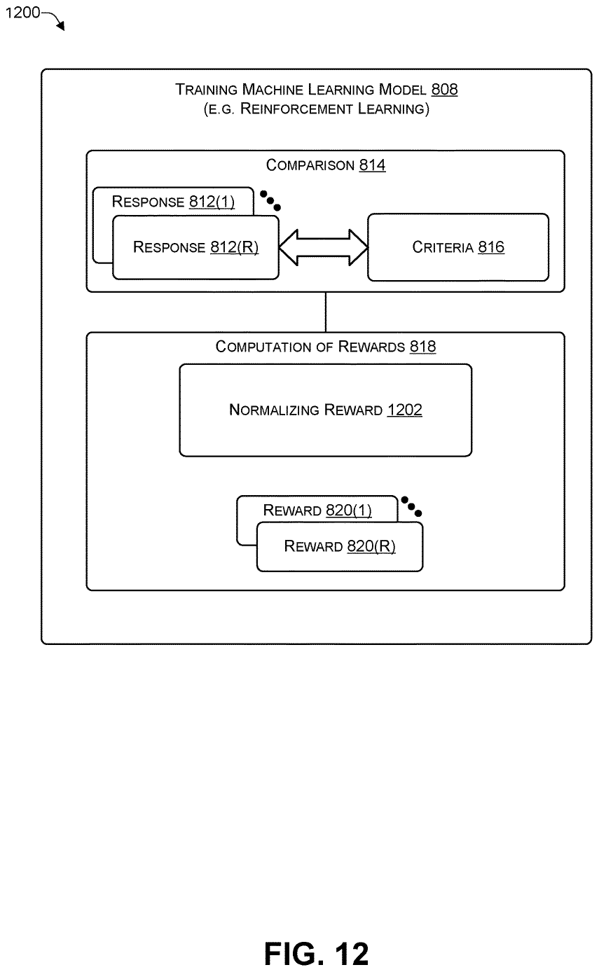

Thus, the output of the second training stage 1002 is the ML model 404, which is the fine-tuned ML model 908 after completion of RL 223 training. The ML model 404 is configured to generate correct and compact compressed reasoning traces 406 in response to input queries 402 at inference time, with the compressed token count 606 that is less than the natural language token count 612 of a natural language reasoning trace 604 for the same input query 402. The ML model 404 produced by the second training stage 1002 generates compressed reasoning traces 406 with the compressed token count 606 that is between 1% and 15% of the natural language token count 612 of the natural language reasoning trace 604, while maintaining Pass@1 accuracy at or near the level of the natural language model 602 baseline. This reduces inference-time computational load, inference latency, and memory consumption of the ML model 404 at inference time. Examples of the reward structures governing the RL 223 training of the second training stage 1002 are described with reference to FIGS. 11 and 12.

FIG. 11 is a block diagram of a system 1100 illustrating the shorter length preference optimization (SLPO) reward structure used in some cases of the second training stage 1002. FIG. 11 describes a conditional reward function 1106 that SLPO applies to a subset of correct responses 1102 to bias the ML model 404 toward generating shorter correct compressed reasoning traces 406 while preserving the accuracy of the correct responses. FIG. 11 shows the subset of correct responses 1102, a conditional reward function 1106, a length preference reward 1108, a uniformly invariant reward 1110, and a baseline reward 1112, as described in detail below.

The system 1100 begins with the training ML model process 808 using RL 223. For individual training prompts 804, the ML model 404 generates a set of responses 812 comprising individual responses 812(1) through 812(R). The system 1100 performs the comparison 814 of individual responses 812(1) through 812(R) against the criteria 816. In some cases, the criteria 816 comprise verifiable correctness based on whether individual final answers 420(1) through 420(R) of individual responses 812(1) through 812(R) match the expected answer in the corresponding training response 806 of the training prompt-response pair 802. Based on the comparison 814, the system 1100 identifies the subset of correct responses 1102. The subset of correct responses 1102 comprise individual correct responses 1104(1) through 1104(S), each of which is a response 812 whose final answer 420 satisfies the criteria 816.

The system 1100 applies the conditional reward function 1106 to compute a reward for individual correct responses 1104(1) through 1104(S). The conditional reward function 1106 applies different reward computations based on a composition of the subset of correct responses 1102. The conditional reward function 1106 applies the length preference reward 1108 when the subset of correct responses 1102 contain more than one correct response with differing lengths, the uniformly invariant reward 1110 when the subset of correct responses 1102 contain only one correct response or all correct responses have the same length, and the baseline reward 1112 when the subset of correct responses 1102 contain no correct responses. The conditional reward function 1106 avoids penalizing uniquely correct responses and avoids distorting the reward signal when no valid solutions exist.

The system 1100 computes the length preference reward 1108 of the compressed reasoning trace 406 of individual correct responses 1104(1) through 1104(S) based on the relative position of the length of that compressed reasoning trace 406 within a range defined by a shortest length and a longest length in the subset of correct responses 1102. Individual correct responses 1104 with a shorter compressed reasoning trace 406 receive a higher length preference reward 1108 than individual correct responses 1104 with a longer compressed reasoning trace 406, for the same training prompt 804. The length preference reward 1108 therefore biases the ML model 404 toward generating shorter correct compressed reasoning traces 406 while preserving accuracy, because only correct responses 1104 are eligible to receive the length preference reward 1108. The system 1100 assigns individual rewards 820 as rewards of correct responses 1114(1) through 1114(S) based on the length preference reward 1108 computed for individual correct responses 1104(1) through 1104(S).

The uniformly invariant reward 1110 applies when the subset of correct responses 1102 contain only one correct response or when all correct responses in the subset of correct responses 1102 have the same length. In this case, no length comparison is possible across the subset of correct responses 1102, and the conditional reward function 1106 assigns a fixed reward to individual correct responses 1104(1) through 1104(S) regardless of their length.

The baseline reward 1112 applies when the subset of correct responses 1102 contain no correct responses, in which case the conditional reward function 1106 assigns a reward of zero to all responses 812 in the set of responses 812. The uniformly invariant reward 1110 and the baseline reward 1112 together ensure that the conditional reward function 1106 handles all possible compositions of the subset of correct responses 1102 without producing undefined or distorted reward signals.

Based on the rewards of correct responses 1114(1) through 1114(S) computed by the conditional reward function 1106, the system 1100 updates the parameters 822 of the ML model 404 to bias it toward generating shorter correct compressed reasoning traces 406 while preserving accuracy of the correct responses. In this way, ML model 404 is trained to generate compressed reasoning traces 406 that achieve the compressed token count 606 (described with reference to FIGS. 4 through 6). By iterating this process across the training prompt-response pairs 802, the ML model 404 learns to generate compressed reasoning traces 406 that are both correct and as short as possible for the input query 402. The SLPO reward structure trains the ML model 404 to generate compressed reasoning traces 406 with compressed token counts 606 of approximately 424 to 971 tokens on mathematical reasoning benchmarks, compared to approximately 1,841 to 7,631 tokens for the natural language reasoning traces 604 of the base model 902, while maintaining Pass@1 at or near the natural language baseline. The SLPO reward structure of FIG. 11 therefore achieves compression of the compressed reasoning trace 406 through the reward signal itself, without imposing a rigid length penalty that would distort the reward landscape or discourage necessary longer reasoning for complex input queries 402.

FIG. 12 is a block diagram of a system 1200 illustrating the GRPO reward structure used in some cases of the second training stage 1002 described with reference to FIG. 10. FIG. 12 describes the GRPO reward structure that applies a normalizing reward 1202 to all responses 812(1) through 812(R) within the set of responses 810 (not shown), but without generating a subset of correct responses 1102 (as with SLPO). The normalizing reward 1202 evaluates individual responses 812 based on their relative position of advantage within the set of responses 810 for individual training prompts 804.

The system 1200 begins with the training ML model process 808 using RL 223, as described with reference to FIG. 8. For individual training prompts 804, the ML model 404 generates the set of responses 810 comprising individual responses 812(1) through 812(R). The system 1200 performs the comparison 814 of individual responses 812(1) through 812(R) against the set of criteria 816. The criteria 816 define the standard against which the system 1200 evaluates individual responses 812. The comparison 814 produces a comparison signal of individual responses 812(1) through 812(R) in the set of responses 810.

Based on the comparison 814, the system 1200 computes the reward 820 of individual responses 812(1) through 812(R), based at least in part on the comparison signal of individual responses 812(1) through 812(R). The computation of rewards 818 produces individual rewards 820(1) through 820(R) corresponding to individual responses 812(1) through 812(R). The system 1200 uses the normalizing reward 1202 to perform the computation of rewards 818.

The normalizing reward 1202 is the distinguishing feature of the GRPO reward structure. The system 1200 computes the normalizing reward 1202 of individual responses 812 based on a relative position of advantage of the compressed reasoning trace 406 of that response 812. The relative position of advantage is within a range defined by a least advantaged and a most advantaged response in the set of responses 810 for the same training prompt 804. The system 1200 computes the relative position of advantage by normalizing individual rewards 820 across the set of responses 810 using a group-relative normalization. The group-relative normalization (i) subtracts the mean reward of the set of responses 810 from the reward of the individual response 812 and (ii) divides by the standard deviation of the rewards of the set of responses 810. Individual responses 812 that are more advantaged relative to other individual responses 812 in the set of responses 810 receive a higher normalizing reward 1202; and individual responses 812 that are less advantaged relative to other individual responses 812 receive a lower normalizing reward 1202.

The normalizing reward 1202 ensures that the system 1200 evaluates individual responses 812 relative to the other individual responses 812 in the same set of responses 810, rather than against an absolute value function. Based on the normalizing rewards 1202 computed as individual rewards 820(1) through 820(R), the system 1200 updates the parameters 822 of the ML model 404. The parameter update 822 biases the ML model 404 toward generating responses 812 that are more advantaged relative to the set of responses 810, thereby biasing the ML model 404 toward generating correct and compact compressed reasoning traces 406 while preserving the accuracy of the correct responses.

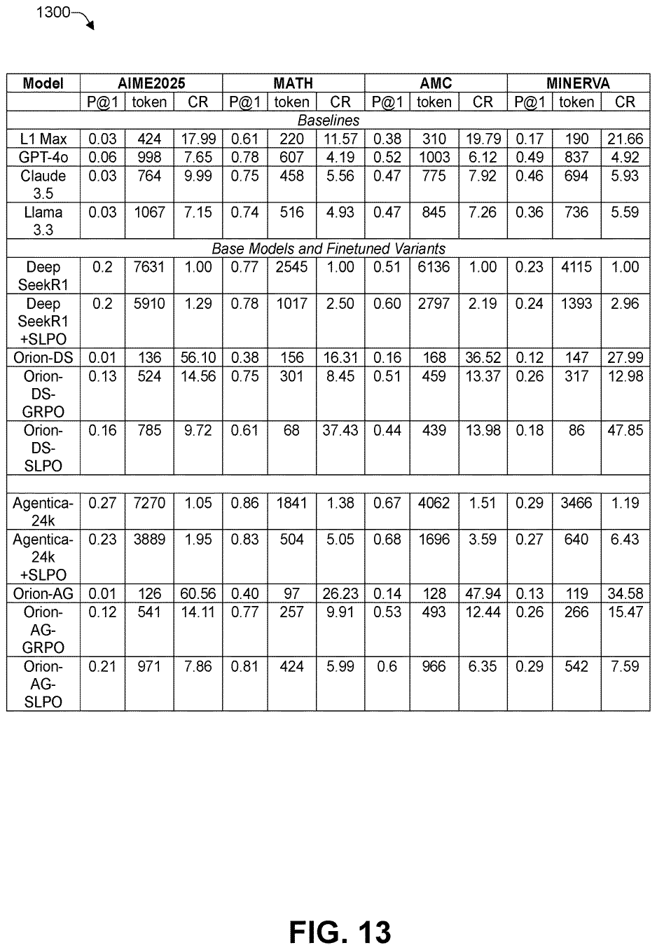

The GRPO reward structure of FIG. 12 and the SLPO reward structure of FIG. 11 represent two cases of the computation of rewards 818 in the second training stage 1002. The SLPO reward structure applies the conditional reward function 1106 only to the subset of correct responses 1102 and uses the length preference reward 1108 to bias the ML model 404 toward shorter correct compressed reasoning traces 406. The GRPO reward structure applies the normalizing reward 1202 to all responses 812(1) through 812(R) in the set of responses 810 and uses group-relative normalization to evaluate the relative quality of individual responses 812 within the set of responses 810. The GRPO reward structure achieves compression rates of approximately 13 to 20 times relative to the natural language token count 612 of the base model 902 across in-domain and out-of-domain benchmarks. This reduces the compressed token count 606 to between 1% and 15% of the natural language token count 612 while maintaining Pass@1 at or near the natural language baseline. In some cases, the GRPO reward structure achieves higher compression rates than the SLPO reward structure, while the SLPO reward structure achieves higher accuracy at moderate compression rates, as illustrated in the benchmark results of FIGS. 13 through 15.

During the second training stage 1002, individual responses 812 are constrained to a maximum generation length during RL 223 training. In implementations applying RL 223 directly to the base model 902, the maximum generation length is set to 8,192 tokens. In implementations applying SLPO to the fine-tuned ML model 908 produced by the first training stage 906, the maximum generation length is set to 2,048 tokens. In implementations cases applying GRPO to the fine-tuned ML model 908, the maximum generation length is set to 1,024 tokens. During evaluation on benchmarks, the maximum generation budget is fixed at 8,192 tokens across all model variants.

FIG. 13 is a table 1300 reporting in-domain benchmark performance of the ML model 404 trained using specified training processes, compared against baseline models (e.g., natural language models 602 or base model 902). The table 1300 reports three metrics for each model: Pass@1 (P@1), token count, and compression rate (CR). The benchmarks are AIME 2025, MATH, AMC, and Minerva. The results directly support the quantitative limitations described herein, including the compressed token count 606 of the compressed reasoning trace 406, the inference-time latency reduction, and the accuracy of the compressed reasoning trace 406 relative to the natural language reasoning trace 604.

The ML model 404 corresponds to the Orion model variants reported in the table 1300. The Orion model variants comprise two families: (i) the Orion-DS family, based on the DeepSeek R1 1.5B base model 902, and (ii) the Orion-AG family, based on the Agentica-24k base model 902. Each family includes three training variants: a SFT-only variant, an SLPO variant, and a GRPO variant. The SFT-only variants, Orion-DS and Orion-AG, correspond to the ML model 404 trained with the first training stage 906 only and without second training stage 1002 (as in FIGS. 9 and 10). The SLPO variants, Orion-DS-SLPO and Orion-AG-SLPO, correspond to the ML model 404 trained using the multi-stage training process 904, the conditional reward function 1106, and the length preference reward 1108 of the SLPO reward structure (as in FIGS. 9 and 11). The GRPO variants, Orion-DS-GRPO and Orion-AG-GRPO, correspond to the ML model 404 trained using the multi-stage training process 904 and the normalizing reward 1202 of the GRPO reward structure (as in FIGS. 9 and 12).

The P@1 metric measures the fraction of input queries 402 for which the specified model produces a correct answer on the first attempt. The token count metric reports the average number of tokens generated per response across all test input queries 402, corresponding directly to the compressed token count 606 of the compressed reasoning trace 406 or to the natural language token count 612 of natural language reasoning trace 604 (as in FIG. 6). The CR metric quantifies response shortening of compressed reasoning trace 406 relative to the natural language reasoning trace 604 of the DeepSeek R1 1.5B base model 902, with a CR metric of 10 indicating that the ML model 404 generates responses that are ten times shorter on average.

The table 1300 reports results for two groups of baseline models. The first group comprises natural language model 602 baselines: L1 Max, GPT-4o, Claude 3.5 Sonnet, and Llama 3.3 70B. GPT-4o, Claude 3.5 Sonnet, and Llama 3.3 70B are frontier-scale natural language models included as reference points. These models generate natural language reasoning traces 604 with natural language token counts 612 ranging from approximately 190 to 1,067 tokens per response across the four benchmarks. The second group comprises the base models 902 and their direct SLPO variants: the DeepSeek R1 1.5B base model 902 and the Agentica-24k base model 902. The DeepSeek R1 1.5B base model 902 generates natural language token counts 612 ranging from approximately 2,545 to 7,631 tokens per response and serves as the primary CR baseline against which the ML model 404 compression rates are measured. The Agentica-24k base model 902 generates natural language token counts 612 ranging from approximately 1,841 to 7,270 tokens per response across the same benchmarks. The direct SLPO variants of the DeepSeek R1 1.5B base model 902 and the Agentica-24k base model 902 apply SLPO directly to the base model 902 without the first training stage 906.

The ML model 404 variants trained using the multi-stage training process 904 generate compressed reasoning traces 406 with compressed token counts 606 substantially less than the natural language token counts 612 of the baseline models. The Orion-AG-SLPO variant generates compressed reasoning traces 406 with token counts of approximately 971, 424, 966, and 542 tokens on AIME 2025, MATH, AMC, and Minerva respectively, with corresponding CR values of 7.86, 5.99, 6.35, and 7.59. The Orion-DS-GRPO variant achieves CR values of 13.10, 8.45, 13.37, and 12.98 on the same benchmarks. The compressed token count 606 of the compressed reasoning trace 406 falls within the range of 1% to 15% of the natural language token count 612 of the natural language reasoning trace 604, reducing the inference-time computational load on the ML model 404 and translating into an inference-time latency that is at least 25% less than the inference-time latency of the natural language model 602.

The ML model 404 maintains P@1 at or near the level of the natural language model 602 baselines despite the lower compressed token count 606 of the compressed reasoning trace 406. The Orion-AG-SLPO variant achieves P@1 of 0.21, 0.81, 0.60, and 0.29 on AIME 2025, MATH, AMC, and Minerva respectively. The Orion-AG-SLPO variant surpasses GPT-4o and Claude 3.5 Sonnet on MATH and AMC while generating compressed reasoning traces 406 with substantially lower token counts. These results are consistent with the compressed reasoning trace 406 having at least 90% of the accuracy of the natural language reasoning trace 604 generated by the natural language model 602 in response to the same input query 402.

Thus, the table 1300 reports that the ML model 404 generates compressed reasoning traces 406 with compressed token counts 606 within the range of 1% to 15% of the natural language token counts 612, while maintaining P@1 at or near the level of the natural language model 602 baselines across four in-domain mathematical reasoning benchmarks.

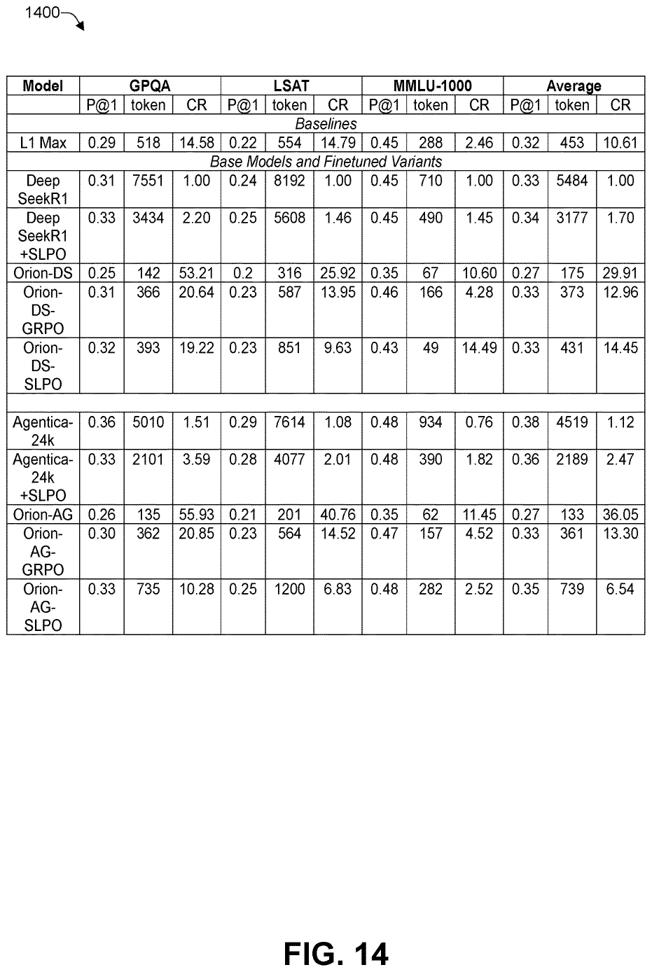

FIG. 14 is a table 1400 reporting out-of-domain (OOD) benchmark performance of the ML model 404 trained using specified training processes, compared against baseline models (e.g., natural language models 602 or base model 902). The table 1400 reports P@1, token count, and CR across three OOD benchmarks: GPQA, LSAT, and MMLU-1000, and an average across all three. The ML model 404 variants, baseline groups, and metrics correspond to those described with reference to FIG. 13. FIG. 14 tests generalization of the multi-stage training process 904 beyond the mathematical reasoning tasks of FIG. 13.

The DeepSeek R1 1.5B base model 902 generates natural language token counts 612 of approximately 7,551, 8,192, and 710 tokens per response on GPQA, LSAT, and MMLU-1000 respectively, and serves as the primary CR baseline. The Agentica-24k base model 902 generates natural language token counts 612 of approximately 5,010, 7,614, and 934 tokens per response on the same benchmarks. The GPQA benchmark tests scientific reasoning, the LSAT benchmark tests legal reasoning, and the MMLU-1000 benchmark tests general knowledge reasoning.

The ML model 404 generates compressed reasoning traces 406 with compressed token counts 606 substantially less than natural language token counts 612 of the natural language reasoning traces 604 generated by the base models 902 across all three OOD benchmarks. The Orion-AG-SLPO variant achieves CR values of 10.28, 6.83, and 2.52 on GPQA, LSAT, and MMLU-1000 respectively. The Orion-DS-GRPO variant achieves CR values of 20.64, 13.95, and 4.28 on the same benchmarks.

The ML model 404 maintains P@1 at or near the level of the natural language model 602 baselines on OOD benchmarks despite the lower token count of the compressed reasoning trace 406 relative to the natural language token count 612 of the natural language reasoning trace 604. The Orion-AG-SLPO variant achieves P@1 of 0.33, 0.25, and 0.48 on GPQA, LSAT, and MMLU-1000 respectively, compared to the DeepSeek R1 1.5B base model 902 P@1 of 0.31, 0.24, and 0.45 on the same benchmarks. The Orion-DS-GRPO variant achieves P@1 of 0.31, 0.23, and 0.46 on GPQA, LSAT, and MMLU-1000 respectively. These results are consistent with the compressed reasoning trace 406 having at least 90% of the accuracy of the natural language reasoning trace 604 generated by the natural language model 602 in response to the same input query 402.

Thus, the table 1400 reports that the ML model 404 generalizes the compressed token count 606 reductions and P@1 results of FIG. 13 to OOD benchmarks across scientific, legal, and general knowledge reasoning tasks.

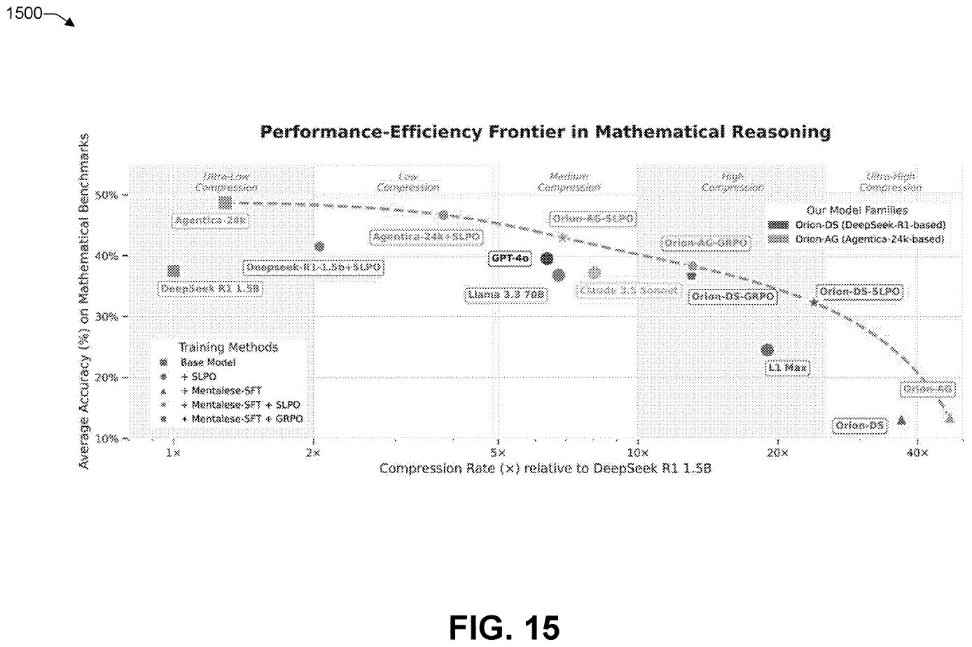

FIG. 15 is a chart 1500 illustrating the performance-efficiency frontier of the ML model 404 trained using specified training processes, compared against baseline models (e.g., natural language models 602 or base model 902). The horizontal axis represents the CR relative to the DeepSeek R1 1.5B base model 902, and the vertical axis represents average Pass@1 (P@1) accuracy across mathematical reasoning benchmarks. The chart 1500 plots individual model variants at their respective CR and P@1 coordinates. A dotted curve indicates the Pareto frontier connecting the ML model 404 variants that achieve the optimal balance between compressed token count 606 and P@1 at each compression level. The ML model 404 variants and baseline models correspond to those described with reference to FIG. 13.

The horizontal axis spans five compression regions: ultra-low, low, medium, high, and ultra-high. A higher CR indicates a lower compressed token count 606 and a correspondingly lower inference-time computational load on the ML model 404. The chart 1500 shows that the ML model 404 operates across multiple compression regions depending on the reward structure applied during the second training stage 1002.

The chart 1500 plots the DeepSeek R1 1.5B base model 902 at a CR of 1 times, representing the natural language token count 612 baseline. The natural language model 602 baselines, including GPT-4o, Claude 3.5 Sonnet, and Llama 3.3 70B, plot in the ultra-low and low compression regions with CR values between 1 times and 5 times, reflecting the natural language token counts 612 of their natural language reasoning traces 604. The L1 Max baseline plots in the high compression region at approximately 20 times CR but at substantially lower P@1 than the ML model 404 variants at comparable compression rates.

The ML model 404 variants trained using the multi-stage training process 904 plot along or near the Pareto frontier across multiple compression regions. The Orion-AG-SLPO variant plots in the low to medium compression region with a CR of approximately 6 to 8 times and a P@1 that surpasses GPT-4o and Claude 3.5 Sonnet at substantially higher CR values. The Orion-DS-GRPO and Orion-AG-GRPO variants plot in the medium to high compression region with CR values of approximately 13 to 15 times while maintaining P@1 at or near the level of the natural language model 602 baselines. The Orion-DS-SLPO variant plots in the ultra-high compression region with CR values of up to 40 times. These positions confirm that the compressed token count 606 of the compressed reasoning trace 406 is within the range of 1% to 15% of the natural language token count 612, as described herein.

The chart 1500 distinguishes model variants by training method: base model 902, direct SLPO, SFT 222 only, SFT 222 plus SLPO, and SFT 222 plus GRPO. The ML model 404 variants trained using the complete multi-stage training process 904 consistently reside on or near the Pareto frontier. The ML model 404 variants trained using direct SLPO without the first training stage 906 do not reach the Pareto frontier, consistent with the training stability analysis described with reference to FIG. 16. The SFT-only variants, Orion-DS and Orion-AG, plot in the ultra-high compression region but at substantially lower P@1 than the SLPO and GRPO variants, reflecting the accuracy drop that the first training stage 906 alone produces without the RLVR 223 recovery of the second training stage 1002.

Thus, the chart 1500 illustrates that the ML model 404 trained using the multi-stage training process 904 plots on the Pareto frontier across multiple compression regions, maintaining P@1 at or near the level of the natural language model 602 baselines, while generating compressed reasoning traces 406 with substantially lower compressed token counts 606. Moreover, the ML model 404 variants achieve compression rates of approximately 6 to 40 times relative to the natural language token count 612 baseline, confirming that the compressed token count 606 falls within the range of 1% to 15% of the natural language token count 612.

FIG. 16 is a set of charts 1600 illustrating the training dynamics of the ML model 404 trained using the multi-stage training process 904, compared against direct SLPO that was applied directly to the base model 902 without the first training stage 906. The charts 1600 plot five metrics across training steps: response length, clip ratio, entropy loss, training time per RL step, and test performance on AIME 2024. The three training configurations compared are (i) SFT 222 plus SLPO 1100, (ii) SFT 222 plus GRPO 1200, and (iii) direct SLPO. The charts 1600 indicate that the first training stage 906 promotes stable and efficient RLVR 223 training in the second training stage 1002.

The response length chart plots the average token number generated per response across training steps in each training configuration. The SFT 222 plus SLPO 1100 and SFT 222 plus GRPO 1200 configurations maintain stable and low response lengths across all training steps, consistent with the ML model 404 generating compressed reasoning traces 406 with the compressed token counts 606. The direct SLPO training configuration collapses at approximately 400 training steps due to gradient instability. The SFT 222 plus GRPO 1200 configuration also experiences training instability, terminating at approximately 800 training steps. This training collapse demonstrates that applying SLPO directly to the base model 902 without the first training stage 906 causes unstable training, and that the first training stage 906 is necessary for stable generation of the compressed reasoning trace 406.

The clip ratio chart plots the fraction of policy gradient updates that the training process clips across training steps. At each training step, the current ML model 404 (having a current policy gradient) is compared against the previous ML model 404 (having a previous policy gradient), and any policy gradient updates that deviate too far from the previous policy are clipped (or capped), preventing large destabilizing parameter updates 822. The SFT 222 plus SLPO 1100 and SFT 222 plus GRPO 1200 configurations maintain lower and more controlled clip ratios than the direct SLPO configuration across earlier training steps. The compressed token count 606 of the compressed reasoning trace 406 keeps responses within the maximum token generation limit, producing complete responses with reliable reward signals and parameter updates 822 within the clipping threshold. The direct SLPO configuration generates longer natural language responses that often hit the maximum token limit, producing higher and more variable clip ratios. After about 100 training steps, the clip ratios of the SFT 222 plus SLPO 1100 and SFT 222 plus GRPO 1200 configurations rise and plateau above the direct SLPO curve, reflecting ongoing learning in which the ML model 404 refines its policy gradient toward shorter correct compressed reasoning traces 406. The direct SLPO clip ratio disappears from the chart after about 100 training steps because it has collapsed (see response length), producing no meaningful policy gradient updates and therefore no meaningful clip ratio signal.

The entropy loss chart plots the entropy of the output distribution of the ML model 404 across training steps. Entropy measures the diversity of the ML model 404 output distribution at a given training step, with higher entropy indicating more exploratory generation and lower entropy indicating more deterministic generation. The SFT 222 plus SLPO 1100 and SFT 222 plus GRPO 1200 configurations have higher entropy loss than the direct SLPO configuration during the first third of training steps. This reflects genuine exploration of the space of valid symbolic reasoning steps 408 before converging to correct and compact compressed reasoning traces 406 (better exploration-exploitation balance). The direct SLPO configuration produces lower entropy early in training, indicating a rapid collapse toward a narrow output distribution (rather than genuine exploration), consistent with the observed training collapse.

The training time per RL step chart plots the computational time required for individual RL training steps. The SFT 222 plus SLPO 1100 and SFT 222 plus GRPO 1200 configurations comprise RL training according to the second training stage 1002 and require substantially lower training time per RL step than the direct SLPO configuration (base model directly trained using SLPO without the first training stage 906). The lower training time per RL step reflects that the first training stage 906 significantly reduces the length of the compressed reasoning trace 406 to the compressed token count 606 (e.g., 10× shorter than natural language reasoning traces 604 of direct SLPO), reducing the computational cost of each training step in the second training stage 1002. The chart confirms that the SFT 222 intermediate stage leads to improved training efficiency (e.g., reduced training costs by 7-10×) and stability in the second training stage 1002.

The test performance chart plots P@1 on the AIME 2024 benchmark across training steps for each of the three training configurations. The SFT 222 plus SLPO 1100 and SFT 222 plus GRPO 1200 configurations achieve P@1 of approximately 22% on AIME 2024 by the end of training. The direct SLPO configuration starts with a higher P@1 than the SFT 222 plus SLPO 1100 configuration, reflecting the strong natural language reasoning capability of the base model 902. As SLPO pressure increases and gradient instability sets in, the direct SLPO configuration collapses, and its P@1 drops sharply as reasoning becomes incomplete or incoherent. The P@1 of SFT 222 plus SLPO 1100 and SFT 222 plus GRPO 1200 configurations are consistent with the compressed reasoning trace 406 having at least 90% of the accuracy of the natural language reasoning trace 604.

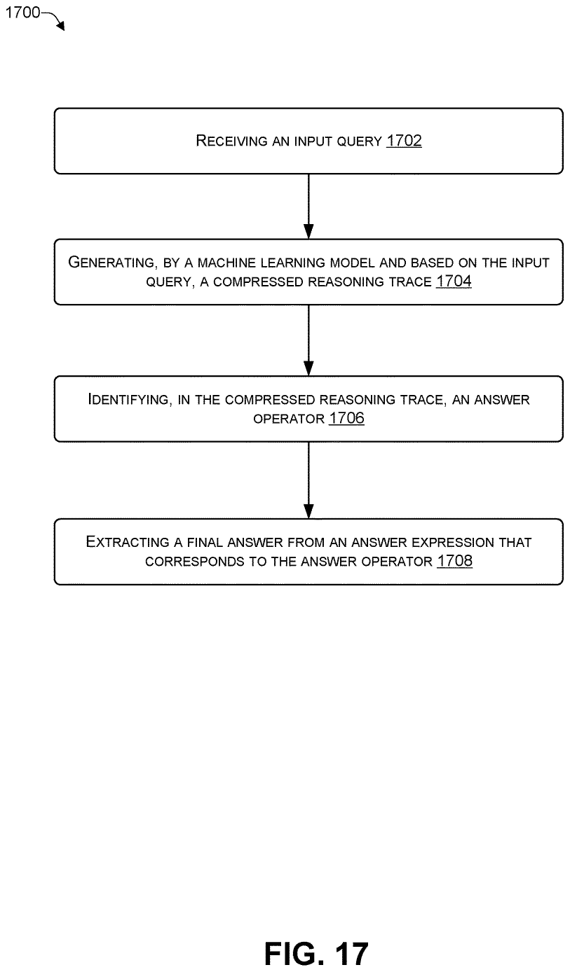

FIG. 17 is a flowchart of a method 1700 illustrating the steps performed by the ML model 404 at inference time to receive the input query 402, generate the compressed reasoning trace 406, identify the answer operator 416, and extract the final answer 420.

At step 1702, the ML model 404 receives the input query 402. The input query 402 is a text-based or natural language question that the ML model 404 resolves.

At step 1704, the ML model 404 generates the compressed reasoning trace 406 based on the input query 402. The compressed reasoning trace 406 comprises the sequence of symbolic reasoning steps 408. Individual symbolic reasoning steps 410 comprise the operator token 412 selected from the set of predefined reasoning operators 502 and the symbolic expression token 414 encoding the logical operation 508 without natural language words. The compressed reasoning trace 406 has the compressed token count 606 that is less than the natural language token count 612 of the natural language reasoning trace 604 produced by the natural language model 602 in response to the same input query 402, reducing inference-time computational load on the ML model 404.

At step 1706, the ML model 404 identifies the answer operator 416 in the compressed reasoning trace 406. The answer operator 416 is a specific operator token selected from the predefined reasoning operators 502 and indicates that the compressed reasoning trace 406 has concluded. As described with reference to FIG. 4, the identification of the answer operator 416 triggers the ML model 404 to terminate generation of the compressed reasoning trace 406.

At step 1708, the ML model 404 extracts the final answer 420 from the answer expression 418 that corresponds to the answer operator 416 in the compressed reasoning trace 406. The answer expression 418 is the symbolic expression token 414 of the terminal symbolic reasoning step 410(N), as described with reference to FIG. 4. The final answer 420 is the output of the ML model 404 for the input query 402.

The method 1700 produces the final answer 420 with the compressed token count 606 substantially less than the natural language token count 612 of the natural language reasoning trace 604, reducing inference-time computational load, inference latency, and memory consumption relative to the natural language model 602.

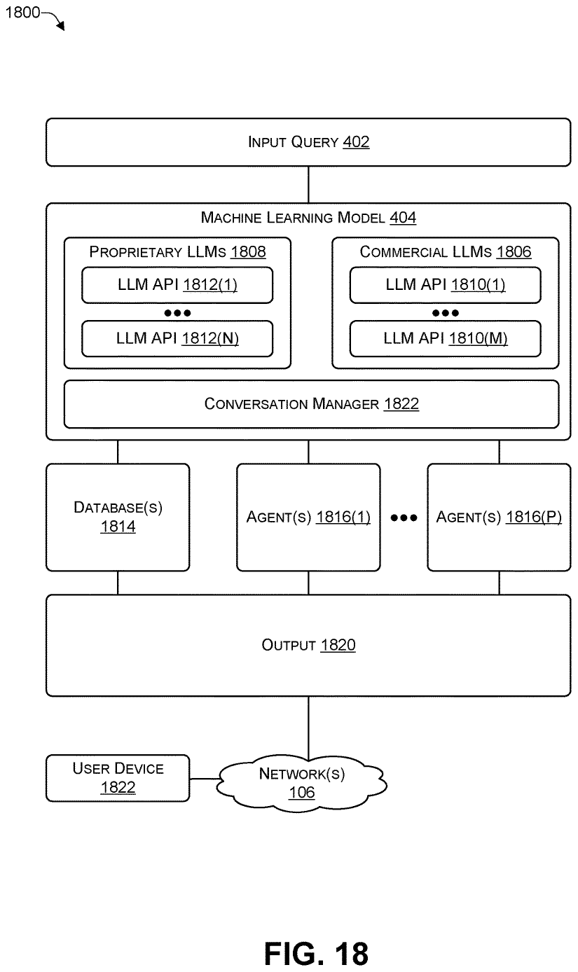

FIG. 18 is a block diagram of system 1800 that includes an LLM routing system, according to some implementations. FIG. 18 illustrates an example conversational AI system, according to some implementations. In some examples, the system 1800 can be implemented using one or more servers that collectively implement processes for enabling examples as described. In variations, system 100 can be implemented in part using client or user devices, such that an architecture described with an example of FIG. 1 is distributed. Further, with reference to FIG. 1, the system illustrates an LLM-based architecture to process system input and generate output. The system 100 can be implemented for a variety of applications, such as, for example, medical-related applications, and applications for implementing bedside manners.

In some examples, the input of system 1800 can be in multiple modalities, such as text, speech, images, audio and video. The system 1800 can accept input from a variety of sources, such as users, sensors, and other systems. Further, the system 1800 may use a commercial large language model (LLM) 1806 to process the input query 402. The commercial LLM 1806 can be used to understand the input query 402, generate text, translate languages, and answer questions. In some examples, the system 1800 may use a proprietary LLM 1808 instead of or in addition to the commercial LLM 1806. The proprietary LLM 1808 can be trained on a specific dataset to improve its performance for a particular task. In some cases, the commercial LLM 1806 or the proprietary LLM 1808, or both, are implemented as the ML model 404 of the present disclosure, generating the compressed reasoning trace 406 comprising a sequence of symbolic reasoning steps 408 without natural language words in response to the input query 402.

In some examples, the system 1800 can use an LLM application programming interface (API) to access the LLMs 1806, 1808. For example, LLM APIs 1810(1) to 1810(M) (M>0) may be used to access commercial LLMs 1806. The LLM APIs 1812(1) to 1812(N) (N>0) may be used to access the proprietary LLMs 1808. The LLM APIs 1810, 1812 can be used to send requests to the LLMs 1806, 1808 and receive responses from the LLMs 1806, 1808. The system 1800 can use a proprietary LLM routing system 1812 to route requests, such as a representative request 1804 to the appropriate LLMs 1806, 1808. The LLM routing system 1812 can be used to improve the performance and efficiency of the system 1800 by routing requests to the LLMs 1806, 1808 that are best suited for the task.

Further, in examples, the system 1800 can use one or more databases 1814 to store data. The data can be stored in a variety of formats, such as text, images, and video. The system 1800 can use a variety of agents 1816(1) to 1816(P) (P>0) to perform tasks. The agents 1816 can be used to generate text, translate languages, answer questions, and perform other tasks. The system 1800 can use safety modules 1818 to prevent the system from performing unsafe actions. The safety modules 1818 can be used to prevent the system 1800 from generating text that is harmful, offensive, illegal, or the like and that complies with various medical standards.

The system 1800 can generate output 1820 in multiple modalities, such as text, speech, images, and video. The output 1820 can be sent to a variety of destinations, such as users, sensors, and other systems. The output of the system 1800 is combined to provide appropriate vocal utterances to the human. In examples, system 1800 implements voice conversations in a healthcare setting or environment. The system 1800 can incorporate multiple LLMs 1806, 1808 and a conversation manager 1822 to facilitate natural language interactions between humans and healthcare providers. The system 1800 includes a front-end interface that is accessible via voice assistants or mobile applications. When a human initiates a conversation, the system uses automatic speech recognition (ASR) technology to transcribe the spoken input and convert it into text. The text is then processed by the conversation manager 1822, which uses advanced natural language understanding (NLU) techniques to identify the intent of the human's request and route it to the appropriate LLM 1806, 1808.

The LLMs 1806, 1808 may be fine-tuned for specific healthcare domains, such as cardiology, radiology, or dermatology, to ensure high accuracy and relevance of the responses. Each LLM 1806, 1808 is trained on a large corpus of healthcare data, including electronic health records, medical literature, and human feedback, to provide a comprehensive knowledge base for the conversation. In some cases, the fine-tuning of the LLMs 1806, 1808 on healthcare data follows the multi-stage training process 904 described with reference to FIGS. 9 and 10, comprising SFT 222 on training prompt-response pairs 802 in which individual training responses 806 are compressed reasoning traces 406, followed by reinforcement learning on verifiable rewards (RLVR) 223 to bias the LLMs 1806, 1808 toward generating shorter correct compressed reasoning traces 406 on medical input queries 402. The system 1800 also includes a set of targeted sub-domain adapter models for medical coding, compliance, pharmacist, nurse practitioner, and dentist. Each domain-specific module has a distinct set of self-auditing correction mechanisms and utilizes separate knowledge retrieval databases.

The conversation manager 1822 coordinates the flow of the conversation between LLM 1806, 1808 and a human by selecting the appropriate LLM 1806, 1808 based on the human's request and ensuring a coherent and engaging dialogue. The system 1800 incorporates conversational strategies, such as open-ended questions, feedback loops, and active listening techniques, to maintain human engagement and satisfaction. The system 1800 implements processes and functionality to ensure that the LLM 1806, 1808 is factually accurate during conversations with humans to build trust, credibility, and reduce anxiety and stress for humans. To ensure human safety and privacy, the system 1800 employs several security and compliance measures. All data can be encrypted and stored securely, and access to the system is restricted to authorized healthcare providers and staff. The system 1800 adheres to relevant healthcare regulations, such as the Health Insurance Portability and Accountability Act (HIPAA) and the General Data Protection Regulation (GDPR).

The system 1800 implements multiple techniques to reduce latency, such as by using cached conversational elements and parallel layered processing of semantic information. Where the LLMs 1806, 1808 are implemented as the ML model 404 of the present disclosure, the compressed token count 606 of the compressed reasoning trace 406 further reduces inference latency relative to a natural language model 602 generating a natural language reasoning trace 604 for the same input query 402, as described with reference to FIG. 6. The system 1800 performs tone detection to identify mood information and self-modulation to respond appropriately. The system 1800 can implement one or more processes to manage dialog by keeping track of existing human information from the database along with new information gathered during the course of the conversation and purposefully accomplishing particular conversational objectives (set an appointment, confirm a prescription is being taken, explain a medical bill, etc.).

Thus, the system 1800 illustrates a multi-modal, multi-LLM conversational AI architecture for healthcare applications in which commercial LLM 1806 and proprietary LLM 1808 are routed by LLM routing system 1812 to process input queries 402 from multiple modalities and generate output 1820. In some cases, in which the LLMs 1806, 1808 are implemented as the ML model 404 of the present disclosure, the compressed token count 606 of the compressed reasoning trace 406 reduces inference latency relative to a natural language model 602. This allows the system 1800 to support real-time voice conversations with reduced response delay and allows the same server hardware to support a higher number of concurrent conversations.

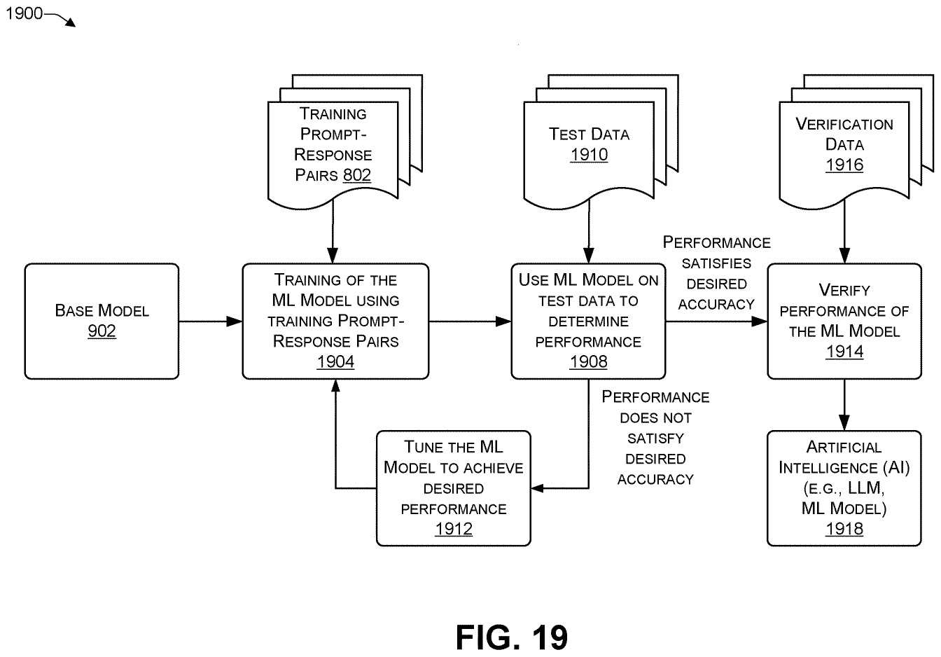

In the flow diagram of FIG. 19, each block represents one or more operations that can be implemented in hardware, software, or a combination thereof. In the context of software, the blocks represent computer-executable instructions that, when executed by one or more processors, cause the processors to perform the recited operations. Generally, computer-executable instructions include routines, programs, objects, modules, components, data structures, and the like that perform particular functions or implement particular abstract data types. The order in which the blocks are described is not intended to be construed as a limitation, and any number of the described operations can be combined in any order and/or in parallel to implement the processes. For discussion purposes, the process 1900 is described with reference to FIGS. 4 through 17 as described above, although other models, frameworks, systems, and environments may be used to implement these processes.

The process 1900 begins with the base model 902. At step 1904, the process 1900 trains the base model 902 using training prompt-response pairs 802. The training prompt-response pairs 802 comprise individual training prompts 804 and their corresponding training responses 806. In some cases, step 1904 corresponds to the multi-stage training process 904 described with reference to FIGS. 9 and 10, comprising SFT 222 in the first training stage 906 followed by RL 223 in the second training stage 1002. In some cases, step 1904 corresponds to the first training stage 906. In some cases, step 1904 corresponds to the second training stage 1002. The first training stage 906 using SFT 222 trains the base model 902 on training prompt-response pairs 802 in which individual training responses 806 are compressed reasoning traces 406 in the symbolic reasoning language. The second training stage 1002 using RL 223 stage trains the fine-tuned ML model 908 to generate shorter and more accurate compressed reasoning traces 406.

At step 1908, the process 1900 tests the ML model 404 using test data 1910 to determine a performance metric. The performance metric may include, for example, precision, recall, Pass@1 (P@1), compressed token count 606, compression rate (CR), or a more complex performance metric. If the performance metric does not satisfy a desired accuracy at step 1908, the process 1900 proceeds to step 1912 to tune the ML model 404. At step 1912, the ML model 404 is further trained to improve its performance, for example by adjusting the SFT 222 training data, the RL 223 reward structure, or both, as described with reference to FIGS. 11 and 12. After step 1912, the process 1900 returns to step 1904. The process 1900 iterates steps 1904, 1908, and 1912 until the ML model 404 satisfies the desired accuracy.

After determining at step 1908 that the ML model 404 satisfies the desired accuracy, the process 1900 proceeds to step 1914 to verify the performance of the ML model 404 using verification data 1916. The verification data 1916 comprises input queries 402 that the ML model 404 has not encountered during training or testing. After the performance of the ML model 404 is verified at step 1914, the ML model 404 is deployed as an artificial intelligence (AI) 1918, such as the LLM 130 or the ML model 404. The AI 1918 receives input queries 402 and generates compressed reasoning traces 406 at inference time, reducing inference-time computational load, inference latency, and memory consumption relative to the natural language model 602.

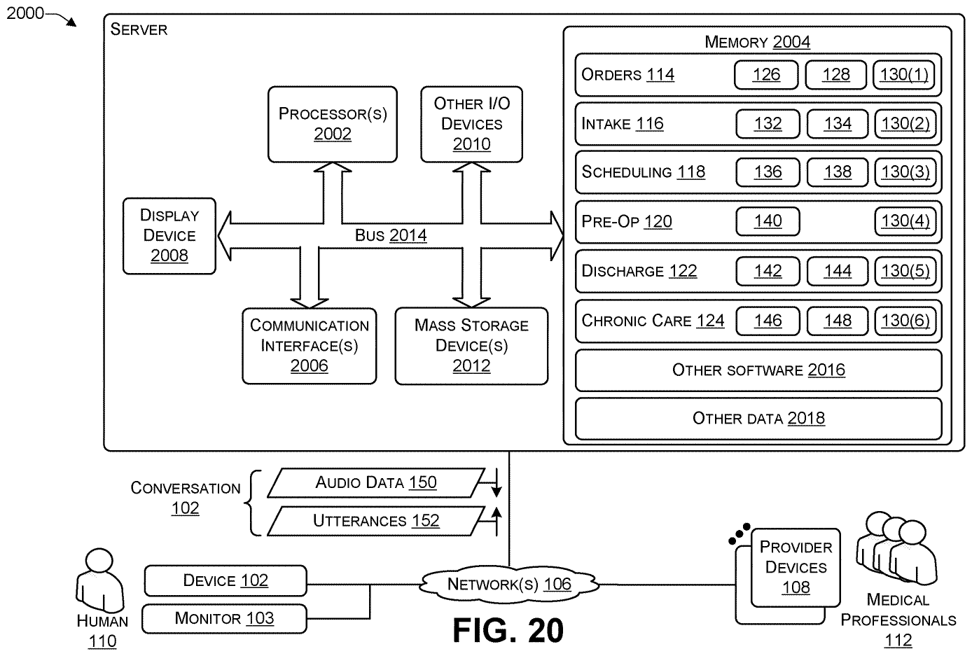

FIG. 20 illustrates an example configuration of a device 2000 that can be used to implement the systems and techniques described herein. For example, the device 2000 may be a server (or a set of servers) used to host one or more of the components described herein. In some cases, the systems and techniques described herein may be implemented as an application programming interface (API), a plugin, or another type of implementation. In some cases, the device 2000 hosts the ML model 404 of the present disclosure and executes the inference-time operations described with reference to FIGS. 4 through 7, and the method 1700, including receiving the input query 402, generating the compressed reasoning trace 406, identifying the answer operator 416, and extracting the final answer 420.

The device 2000 may include one or more processors 2002 (e.g., central processing unit (CPU), graphics processing unit (GPU), or the like), a memory 2004, communication interfaces 2006, a display device 2008, other input/output (I/O) devices 2010 (e.g., keyboard, trackball, and the like), and one or more mass storage devices 2012 (e.g., disk drive, solid state disk drive, or the like), configured to communicate with each other, such as via one or more system buses 2014 or other suitable connections. While a single system bus 2014 is illustrated for ease of understanding, it should be understood that the system bus 2014 may include multiple buses, such as a memory device bus, a storage device bus (e.g., serial ATA (SATA) and the like), data buses (e.g., universal serial bus (USB) and the like), video signal buses (e.g., ThunderBolt®, digital video interface (DVI), high definition media interface (HDMI), and the like), power buses, etc.

The processors 2002 are one or more hardware devices that may include a single processing unit or a number of processing units, all of which may include single or multiple computing units or multiple cores. The processors 2002 may include a graphics processing unit (GPU) that is integrated into the CPU or the GPU may be a separate processor device from the CPU. The processors 2002 may be implemented as one or more microprocessors, microcomputers, microcontrollers, digital signal processors, central processing units, graphics processing units, state machines, logic circuitries, and/or any devices that manipulate signals based on operational instructions. Among other capabilities, the processors 2002 may be configured to fetch and execute computer-readable instructions stored in the memory 2004, mass storage devices 2012, or other computer-readable media.

In some cases, the memory 2004 stores the parameters of the ML model 404 trained using the multi-stage training process 904, including the parameters updated by the first training stage 906 (e.g., SFT 222) and/or the second training stage (e.g., RL 223). The memory 2004 also stores the training prompt-response pairs 802 and the compressed reasoning traces 406 generated during inference. The reduced compressed token count 606 of the compressed reasoning trace 406 relative to the natural language token count 612 of the natural language reasoning trace 604 reduces the memory consumption of the ML model 404 during inference on the device 2000.

Memory 2004 and mass storage devices 2012 are examples of computer storage media (e.g., memory storage devices) for storing instructions that can be executed by the processors 2002 to perform the various functions described herein. For example, memory 2004 may include both volatile memory and non-volatile memory (e.g., random access memory (RAM), read only memory (ROM), or the like) devices. Further, mass storage devices 2012 may include hard disk drives, solid-state drives, removable media, including external and removable drives, memory cards, flash memory, floppy disks, optical disks (e.g., compact disc (CD), digital versatile disc (DVD), a storage array, a network attached storage (NAS), a storage area network (SAN), or the like. Both memory 2004 and mass storage devices 2012 may be collectively referred to as memory or computer storage media herein and may be any type of non-transitory media capable of storing computer-readable, processor-executable program instructions as computer program code that can be executed by the processors 2002 as a particular machine configured for carrying out the operations and functions described in the implementations herein.

The device 2000 may include one or more communication interfaces 2006 for exchanging data via the network 110. The communication interfaces 2006 can facilitate communications within a wide variety of networks and protocol types, including wired networks (e.g., Ethernet, Data Over Cable Service Interface Specification (DOCSIS), digital subscriber line (DSL), Fiber, universal serial bus (USB) etc.) and wireless networks (e.g., wireless local area network (WLAN), global system for mobile (GSM), code division multiple access (CDMA), 802.11, Bluetooth, Wireless USB, ZigBee, cellular, satellite, etc.), the Internet and the like. Communication interfaces 2006 can also provide communication with external storage, such as a storage array, network attached storage, storage area network, cloud storage, or the like.

The display device 2008 may be used for displaying content (e.g., information and images) to users. Other I/O devices 2010 may be devices that receive various inputs from a user and provide various outputs to the user, and may include a keyboard, a touchpad, a mouse, a gaming controller (e.g., joystick, steering controller, accelerator pedal, brake pedal controller, virtual reality (VR) headset, VR glove, or the like), a printer, audio input/output devices, and so forth.

The computer storage media, such as memory 2004 and mass storage devices 2012, may be used to store any of the software and data described herein.

Thus, the device 2000 illustrates an example hardware configuration for hosting the ML model 404 of the present disclosure. The reduced compressed token count 606 of the compressed reasoning trace 406 relative to the natural language token count 612 of the natural language reasoning trace 604 reduces the number of token-generation steps executed by processors 2002 and memory consumption of memory 2004 during inference on the device 2000. This allows the device 2000 to host the ML model 404 with lower hardware requirements than would be needed to host the natural language reasoning model 602 of equivalent parameter count.

The example systems and computing devices described herein are merely examples suitable for some implementations and are not intended to suggest any limitation as to the scope of use or functionality of the environments, architectures and frameworks that can implement the processes, components and features described herein. Thus, implementations herein are operational with numerous environments or architectures, and may be implemented in general purpose and special-purpose computing systems, or other devices having processing capability. Generally, any of the functions described with reference to the figures can be implemented using software, hardware (e.g., fixed logic circuitry) or a combination of these implementations. The term “module,” “mechanism” or “component” as used herein generally represents software, hardware, or a combination of software and hardware that can be configured to implement prescribed functions. For instance, in the case of a software implementation, the term “module,” “mechanism” or “component” can represent program code (and/or declarative-type instructions) that performs specified tasks or operations when executed on a processing device or devices (e.g., CPUs or processors). The program code can be stored in one or more computer-readable memory devices or other computer storage devices. Thus, the processes, components and modules described herein may be implemented by a computer program product.

Furthermore, this disclosure provides various example implementations, as described and as illustrated in the drawings. However, this disclosure is not limited to the implementations described and illustrated herein, but can extend to other implementations, as would be known or as would become known to those skilled in the art. Reference in the specification to “one implementation,” “this implementation,” “these implementations” or “some implementations” means that a particular feature, structure, or characteristic described is included in at least one implementation, and the appearances of these phrases in various places in the specification are not necessarily all referring to the same implementation.

Although the present technology disclosed has been described in connection with several implementations, the technology disclosed is not intended to be limited to the specific forms set forth herein. On the contrary, it is intended to cover such alternatives, modifications, and equivalents as can be reasonably included within the scope of the technology disclosed as defined by the appended claims.

### Language Models and Artificial Intelligence Agents

The following detailed description of the figures provides a technical exposition of LLM-based agents, language modeling methodologies, enhancement techniques, integration protocols, and computational infrastructure. Large language model-based agents differ fundamentally from backpropagation-driven deep learning systems because their core behavior is governed not primarily by parameter updates, but by inference-time control structures that operate through prompting, contextual conditioning, and dynamic retrieval. Whereas legacy deep learning models rely on supervised datasets, gradient descent, and static weight modification to effect behavioral change, LLM-based agents adapt their operation through system prompts, hierarchical instruction scaffolds, RAG pipelines, low-rank adaptation (LoRA) overlays, Model Context Protocol (MCP) tool interfaces, chain-of-thought reasoning, self-consistency decoding, reinforcement learning from human feedback (RLHF), plan-and-execute orchestration, introspection mechanisms, multi-modal grounding, session-specific memory embeddings, and distributed multi-agent coordination frameworks.

An AI agent (also referred to as an agent or an artificial intelligence agent) is a stateful control architecture that models perception, decision, and action over an environment. It uses one or more LLMs as inference engines inside its policy but augments them with persistent state, memory subsystems, tool interfaces, orchestration logic, and policy enforcement mechanisms that are not part of the underlying language model itself.

In LLM-native systems, the primary locus of computation shifts from offline training to runtime orchestration. System prompts define persistent operational policies; RAG injects real-time, externally maintained knowledge into the model's attention space; LoRA modules provide orthogonal, dynamically loadable specialization layers; and MCP schemas and tool APIs expose external applications and services as structured objects in the model's output vocabulary. Chain-of-thought and self-consistency mechanisms support multi-step internal deliberation and verification prior to committing to actions, while introspection tools enable runtime evaluation and error correction. RLHF further guides adaptive, context-sensitive, reward-aligned behavior.

Multi-modal grounding integrates visual, auditory, or structured interface embeddings with textual instructions to generate perceptually aligned action tokens. Session-specific memory stores, retrieval mechanisms, and summarization policies preserve long-horizon task continuity. Concurrent and distributed agent coordination is achieved through model-mediated message tokens and concurrency-control directives, enabling multiple agents to negotiate, resolve dependencies, and execute collaborative workflows in real time. Collectively, these mechanisms enable adaptive, tool-conditioned, continuously evolving behavior that emerges from inference-time context and structured memory rather than from weight-space optimization, establishing a computational paradigm that is categorically distinct from training-centric deep learning architectures and centered on inference-time generative, autonomous agency.

The figures illustrate the evolution of language models from statistical approaches to contemporary large language models, and in particular to LLM-based agents. The description includes prompt-engineering techniques that structure input to optimize model behavior through system prompts defining roles, guidelines, and constraints. Subsequent description and corresponding figures present RAG systems that integrate external knowledge sources with generative models through retriever and generator components, as well as agentic intelligence architectures in which autonomous agents employ memory systems, planning mechanisms, and tool access to enable dynamic decision-making.

The figures further depict inference hardware structures comprising memory modules and computing hardware for neural network operations, with deployment configurations ranging from edge-device systems to distributed data center architectures. Detailed exposition is provided for the Model Context Protocol (MCP), a standardized communication specification enabling AI applications to access external data sources and services through MCP hosts, clients, and servers that expose tools, resources, and prompts. The integrated system architectures that combine prompt engineering, MCP-orchestrated context retrieval, and Low-Rank Adaptation (LoRA) for parameter-efficient model customization are also presented. The description specifies component functionalities, operational workflows, communication patterns, and architectural principles in sufficient detail to enable understanding and implementation by persons having ordinary skill in the art.

### Language Models

Language modeling (LM) is a primary technique used to advance the language intelligence of machines. The development of language models can be divided into four main stages: statistical language models, neural language models, pre-trained language models, and LLMs.

Statistical language models were primarily used in specific tasks (e.g., retrieval processing) in which predicted or estimated token probabilities improved the performance of task-specific pipelines. Neural language models shifted focus toward learning task-agnostic representations (e.g., feature embeddings), thereby reducing the need for manual feature engineering. Pre-trained language models learned context-aware representations that could be fine-tuned or otherwise optimized for downstream tasks.

The latest generation, LLMs, extend these advances by exploiting scaling effects in model capacity (e.g., parameter count, context window size, and training corpus size), and by leveraging transformer-based architectures. In practice, LLMs function as general-purpose task solvers that can be adapted to a broad range of applications via prompting, lightweight adaptation, and orchestration, rather than by task-specific architectural redesign.

### Prompt

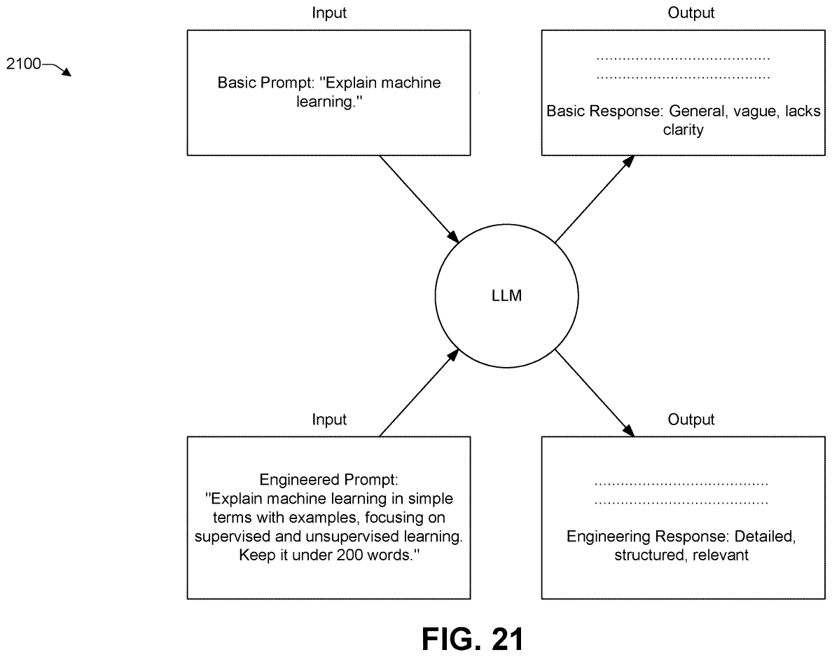

FIG. 21 illustrates a comparison between responses generated by a large language model when provided with a basic prompt and when provided with a more sophisticated, engineered prompt. This comparison, as shown in 2100, highlights the importance of prompt engineering. A basic prompt tends to produce a basic, often vague response, whereas an engineered prompt tends to yield a more structured, detailed, and higher-quality response. The ML model 404 of the present disclosure receives the input query 402 and benefits from prompt engineering in the same manner: an engineered input query 402 that specifies the expected symbolic reasoning format of the compressed reasoning trace 406 and the predefined reasoning operators 502 produces a more compact and accurately structured compressed reasoning trace 406 than input query 402 that is unstructured.

Prompting is a widely used method for leveraging LLMs to solve diverse tasks. The process of creating an effective prompt is referred to as prompt engineering. Well-structured and context-rich prompts enable LLMs to generate more accurate, relevant, and detailed responses, while poorly specified prompts often result in unstructured, ambiguous, or incomplete outputs. Prompt engineering therefore improves the accuracy, coherence, and factual consistency of LLM responses.

A broad spectrum of prompting techniques is available. At one end of the spectrum, zero-shot prompting (e.g., “Summarize this”) provides minimal contextual guidance. At the other end, comprehensive prompt-engineering frameworks supply highly structured prompting strategies, incorporating explicit roles, constraints, examples, and multi-step reasoning scaffolds.

Few-shot prompting lies between these extremes and often yields substantial improvements in response quality. In few-shot prompting, the prompt includes a small number of task examples (input-output pairs), also referred to as few-shot demonstrations. These demonstrations help the LLM internalize the semantic mapping between input and output for the task at hand without requiring parameter updates.

An example of a few-shot prompt is shown below:

[
convert each word into its number of letters
Example 1:
Word: “Tree”
Output: 4
Example 2:
Word: “House”
Output: 5
Example 3:
Word: “Ant”
Output: 3
Now complete
Word: “Planet”
Output:
]

Formally, let Dₖ={f(x₁, y₁), . . . , f(xₖ,yₖ)}represent a set of demonstrations with k examples, where f(xₖ, yₖ)) is the prompt function that transforms the k-th task example into natural language prompts. Given the task description I, demonstration Dₖ, and a new input query xₖ₊₁, the prediction of the output ŷₖ₊₁ generated from LLMs can be formulation as follows:

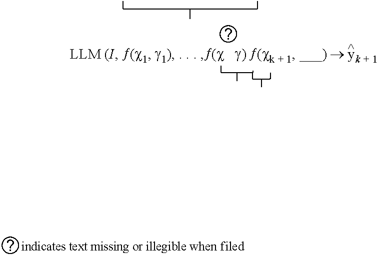

### Demonstrations Input Answer

Where the actual answer yₖ₊₁ is left as blank to predicted by the LLM. In this formulation, the demonstrations serve as in-context training signals, the new input forms the query, and the predicted answer is generated by the model at inference time.

Few-shot prompting is commonly used across a variety of natural language processing (NLP) applications, such as those listed below. In some cases, few-shot demonstrations provided to the ML model 404 of the present disclosure include example input queries 402 paired with their corresponding compressed reasoning traces 406, training the model to produce symbolic reasoning steps 408 in the correct operator-token-plus-expression-token format at inference time without additional parameter updates.
Text Classification: “Classify this new review based on the given positive and negative examples.”
Machine Translation: “Translate these English sentences into Spanish. Now translate the following text.”
Code Generation: “Complete the missing function given these input-output pairs.”
Question Answering: “Answer factual questions based on the provided example Q&A pairs.”

### System Prompt

In some cases, the input query 402 supplied to the ML model 404 of the present disclosure is engineered using one or more of the prompting techniques described above with reference to FIG. 21. For example, a few-shot prompt may provide the ML model 404 with example input queries and their corresponding compressed reasoning traces 406, orienting the model toward generating compact symbolic reasoning steps before producing the final answer 420. A system prompt may further define the role of the ML model 404, specify the symbolic reasoning format of the compressed reasoning trace 406, and constrain the output vocabulary to the predefined reasoning operators 502. Because the compressed reasoning trace 406 has the compressed token count 606 substantially less than a natural language reasoning trace 604, the ML model 404 generates responses to engineered prompts with lower inference latency and lower memory consumption than a natural language model receiving the same engineered prompt.

System prompts are predefined instructions that guide an LLM's behavior when responding to user queries and thus play a critical role in the effective performance and functionality of an LLM. A system prompt defines the model's role, behavioral constraints, objectives, and style, thereby acting as a soft form of policy shaping within the model's learned distribution. In some cases, a system prompt supplied to the ML model 404 of the present disclosure specifies the symbolic reasoning language as the required output format, instructs the ML model 404 to encode each reasoning step using an operator token selected from the predefined reasoning operators 502, and instructs the ML model 404 to terminate the compressed reasoning trace 406 with the answer operator 416, thereby reinforcing the structural constraints that produce the compressed token count 606.

Operationally, the system prompt functions as input-side control: it steers the model by modifying the contextual information on which the model conditions, without altering the underlying model parameters. System prompts may include proprietary or sensitive information about the organization that owns or operates the model, such as private system instructions, proprietary guidelines, functionality descriptions, architectural details, limitations, permission disclosures, role definitions for different classes of users, and basic safety-guardrail configurations.

An example system prompt is provided below:
“You are a travel itinerary assistant. You will help users create personalized trip plans based on their preferences and input regarding destination, budget, interests, and time constraints. Ensure that each itinerary includes essential details, such as accommodation options, transportation methods, key attractions, dining options, and free-time activities. Consider factors like user preferences for pace, specific requests for cultural experiences, or outdoor adventures if mentioned. Use up-to-date information about the destinations and include safety tips where necessary. Make sure that each itinerary is well-balanced, reasonable in terms of time, and enjoyable for the user.”
Guidelines associated with this system prompt may include:
Always prioritize user-driven preferences for destinations and activities.
Deliver a balance between exploration and relaxation within the itinerary.
Offer insights into local culture and practices relevant to the destination.
Help users maximize value for money in booking and planning.
Create itineraries that provide satisfying and meaningful experiences for both family and individual travelers.

### Retrieval-Augmented Generation (RAG)

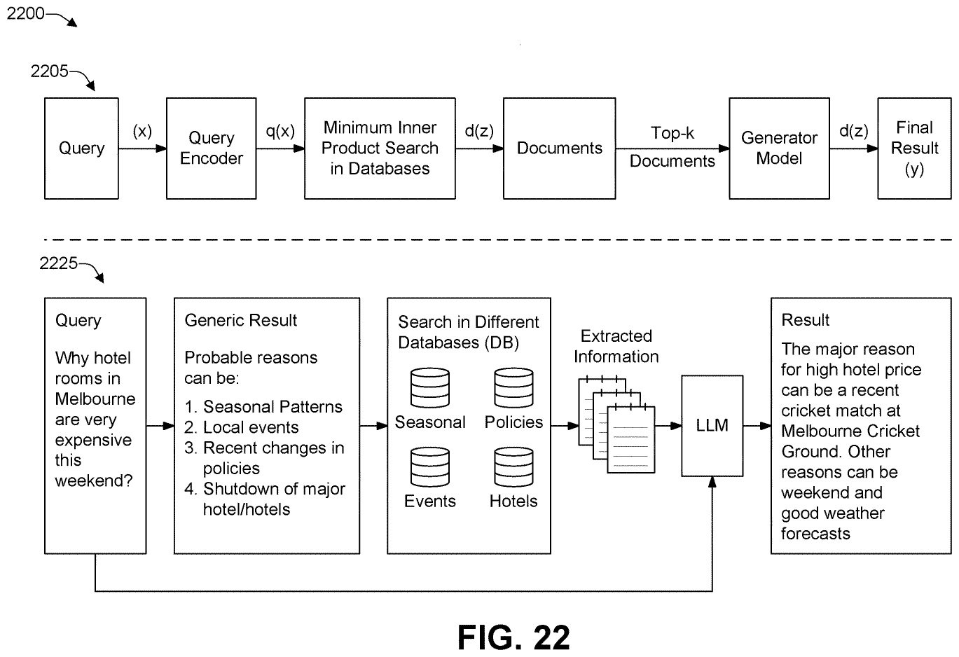

FIG. 22 illustrates a high-level overview of a Retrieval-Augmented Generation (RAG) workflow. RAG is a technique that enhances the capabilities of large language models by combining their generative power with the ability to access and retrieve information from external knowledge sources. This hybrid approach addresses a key limitation of traditional LLMs, which are constrained by the static nature of their training data. While LLMs excel at generating coherent and contextually relevant text, they may lack access to the most recent information or to specialized knowledge that is not contained in their training corpus.

RAG bridges this gap by combining the strengths of retrieval-based systems and generative models. During the generation process, a RAG system first retrieves relevant documents or information from an external knowledge source, such as a database, knowledge graph, or document collection. The retrieved information is then used to augment the input provided to the LLM, enabling the model to generate responses grounded in factual and contextually relevant knowledge. For example, in open-domain question answering, a RAG system can retrieve relevant passages from a large corpus of documents and then use them as context for generating accurate and detailed answers.

A typical RAG framework comprises two primary components: a retriever and a generator. The retriever identifies and fetches the most relevant information from the external knowledge source, while the generator (the LLM) incorporates this retrieved information into a coherent and contextually appropriate response. This combination allows RAG systems to perform tasks that require broad knowledge and precise reasoning, such as answering complex questions, summarizing lengthy documents, or providing grounded explanations. In some cases, the ML model 404 of the present disclosure serves as the generator component, receiving an augmented input query 402 that combines the user's original query with retrieved context and generating the compressed reasoning trace 406 in response. Because the compressed reasoning trace 406 has a compressed token count 606 substantially less than the natural language token count 612 of the natural language reasoning trace 604, the ML model 404 reduces inference latency and memory consumption per generation call within the RAG pipeline relative to a natural language model serving the same generator role.

One of the principal advantages of RAG is its ability to handle knowledge-intensive tasks with improved accuracy and relevance. By leveraging external knowledge sources, RAG helps ensure that generated responses are not only fluent but also factually accurate and up to date. This makes RAG particularly valuable in domains such as customer support, medical diagnosis, and legal research, where factual precision and access to specialized knowledge are critical.

FIG. 22 presents a high-level schematic of RAG operation 2200. A top portion of FIG. 22, labeled 2205, illustrates the RAG framework and the retrieval and generation phases for enhancing LLM responses with external knowledge as described above. A bottom portion of FIG. 22, labeled 2225, presents an example use case demonstrating how retrieved content is combined with a user query to produce a grounded response. In some cases, the generator depicted in both portions of FIG. 22 is implemented as the ML model 404 of the present disclosure, which receives the combined retrieved content and user query as the input query 402 and generates the compressed reasoning trace 406 comprising a sequence of symbolic reasoning steps 408, extracting the final answer 420 from the answer expression 418 corresponding to the answer operator 416.

In some cases, the ML model 404 of the present disclosure is implemented as the generator component of the RAG framework described with reference to FIG. 22. In that configuration, the ML model 404 receives an augmented input query 402 that combines the user's original query with context retrieved from an external knowledge source and generates the compressed reasoning trace 406. Because the compressed reasoning trace 406 has the compressed token count 606 substantially less than the natural language reasoning trace 604, the ML model 404 serving as the RAG generator reduces inference latency and memory consumption per query relative to a natural language model serving the same role, enabling the RAG system to respond to a higher throughput of input queries 402 within the same hardware budget.

### Agentic Intelligence

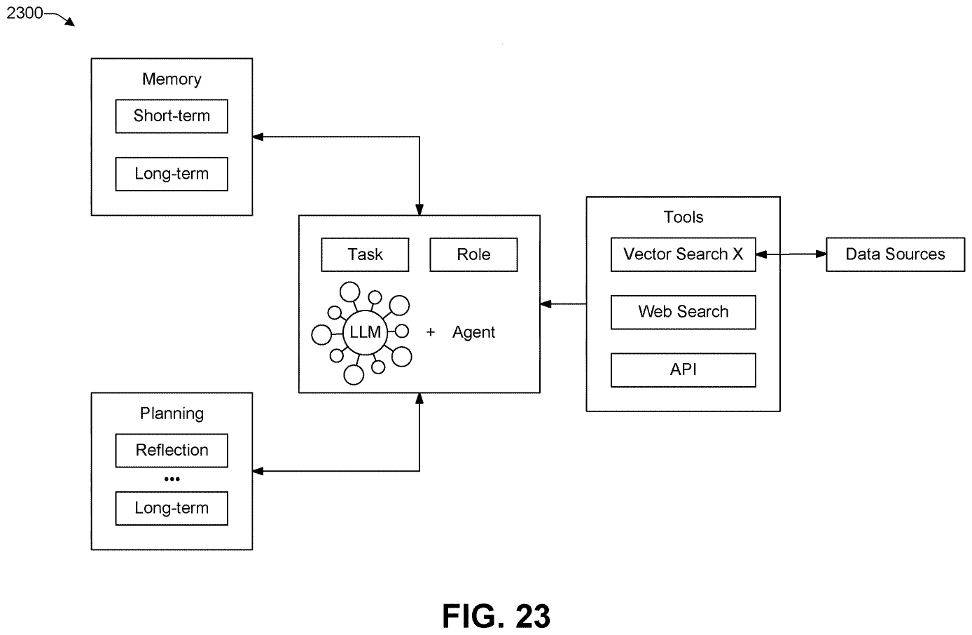

FIG. 23 illustrates an overview of artificial intelligence (AI) agents. Agentic intelligence forms the foundation of Agentic Retrieval-Augmented Generation (Agentic RAG) systems, enabling them to move beyond the static and reactive behavior of conventional RAG pipelines. By integrating autonomous agents capable of dynamic decision-making, iterative reasoning, and collaborative workflows, Agentic RAG systems exhibit enhanced adaptability, robustness, and precision. In some cases, the ML model 404 of the present disclosure serves as the LLM reasoning engine of an AI agent 2300, generating the compressed reasoning trace 406 after receiving each input query 402 routed by the agent's planning component. The reduced token count of the compressed reasoning trace 406 reduces per-turn inference latency, enabling the AI agent 2300 to complete more reasoning cycles within a given compute budget.

In essence, and as depicted in FIG. 23, the AI agent 2300 comprises:
LLM with defined Role and Task: The LLM serves as the agent's primary reasoning engine and dialogue interface. It interprets user queries, executes internal reasoning, generates responses, and maintains conversational coherence. In some cases, the LLM in this role is implemented as the ML model 404 of the present disclosure, generating the compressed reasoning trace 406 comprising the sequence of symbolic reasoning steps 408 for each input query 402, and extracting the final answer 420 from the answer operator 416 to produce each agent response.
Memory (Short-Term and Long-Term): Memory subsystems capture context and relevant data across interactions. Short-term memory tracks the immediate conversational state and local task context, while long-term memory stores accumulated knowledge, user preferences, and prior agent experiences that can be reused across sessions.
Planning (Reflection and Self-Critique): A planning component guides the agent's iterative reasoning process through reflection, query routing, error analysis, or self-critique. This component enables the agent to decompose complex tasks into intermediate steps, evaluate alternatives, and refine strategies over multiple interaction turns.
Tools (Vector Search, Web Search, APIs, etc.): Tool interfaces extend the agent's capabilities beyond pure text generation by enabling access to external resources, real-time data, and specialized computations (e.g., vector search over embeddings, web search, database access, or domain-specific APIs).

### Agentic RAG

Agentic RAG combines the capabilities of agentic AI and RAG. In an Agentic RAG system, autonomous, goal-oriented software agents dynamically retrieve, select, and process information. These agents can perform query routing, step-by-step planning, and decision-making, and can invoke other software tools, services, or agents to obtain more accurate or more complete results.

Agentic RAG systems typically maintain explicit memory structures for planning and for recalling previous prompts, user attributes, or session state. This design improves scalability (by orchestrating multiple tools and agents), flexibility (by dynamically selecting strategies based on context), and context awareness (by grounding retrieval and generation in a persistent memory and tool environment).

Key characteristics of Agentic RAG include:
Autonomous Decision-Making: Agents independently evaluate and manage retrieval strategies based on query complexity, task objectives, and prior outcomes.
Iterative Refinement: Feedback loops are incorporated to refine retrieval results and generated responses, improving accuracy and relevance over multiple iterations.
Workflow Optimization: Tasks are orchestrated dynamically, enabling efficient execution in real-time or near-real-time applications, and allowing the system to adapt to resource constraints and latency requirements. In some cases, this optimization is enhanced by implementing the LLM reasoning engine of the Agentic RAG system as the ML model 404 of the present disclosure, whose compressed token count 606 reduces the per-turn inference latency of each reasoning cycle, enabling more retrieval-and-generation iterations within a given time budget.

In some cases, the ML model 404 of the present disclosure serves as the LLM reasoning engine of the AI agent 2300 of the type described with reference to FIG. 23. In that configuration, the agent's planning component routes input queries 402 to the ML model 404, which generates the compressed reasoning trace 406. The reduced token count of the compressed reasoning trace 406 reduces per-turn inference latency, enabling the AI agent 2300 to complete more reasoning cycles within a given time budget and improving real-time responsiveness of agentic workflows, including Agentic RAG pipelines that rely on iterative retrieval and generation.

### Inference Hardware

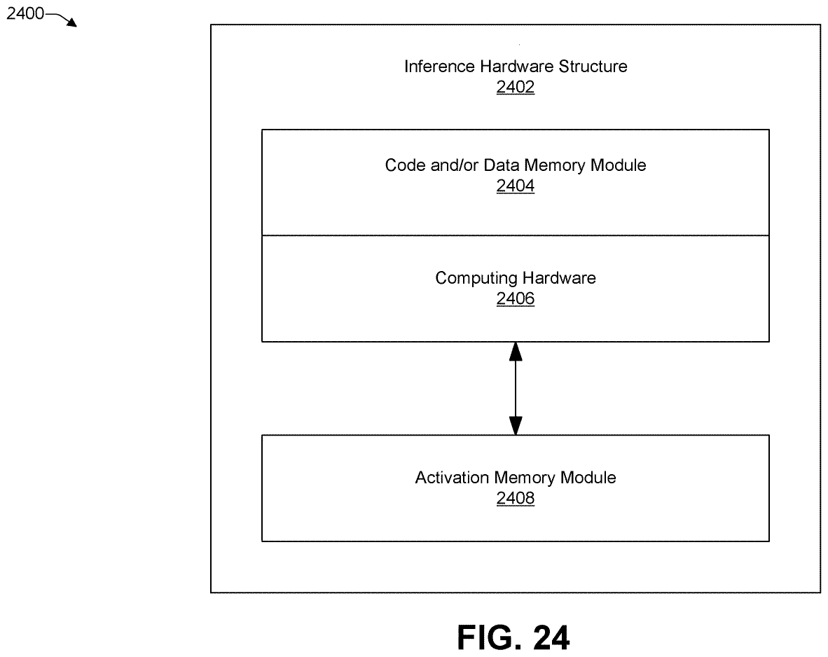

The lifecycle of an AI model generally involves two distinct phases: a training phase, during which model weights are optimized against a large body of training data, and an inference phase, during which the trained model responds to user prompts or other inputs. In the inference phase, the model is “frozen” (i.e., its weights are fixed) and used to generate outputs, such as predicted token sequences, after receiving prompts or context. The ML model 404 of the present disclosure is trained using the multi-stage training process 904 described with reference to FIGS. 9 and 10, and at inference time generates the compressed reasoning trace 406 comprising a sequence of symbolic reasoning steps 408 in response to each input query 402. As model sizes and context windows have increased, the computational and memory requirements during inference have grown substantially, shifting design considerations toward efficiency, low latency, and high throughput. As shown in FIG. 24, an exemplary system 2400 for AI model inference is described below.

FIG. 24 shows an inference hardware structure 2402 used to perform inferencing operations in at least one implementation. Inference hardware structure 2402 is described with reference to FIG. 24.

Inference hardware structure 2402 may include a code and/or data memory module 2404 for storing trained model weights, input/output data, and other parameters used to configure neurons or layers of a trained neural network during inference. Inference hardware structure 2402 can include, or be coupled to, code and/or data memory module 2404 for storing graph code or additional software that controls the timing and order in which inference logic is configured. For example, inference logic may be configured by loading weights and other parameters in low-precision numerical formats such as FP16, BF16, FP8, INT8, or INT4.

Code and/or data memory module 2404 can be associated with a dedicated computing resource, such as computing hardware 2406. Computing hardware 2406 may comprise one or more processors that perform mathematical functions, such as linear algebraic operations and matrix computations. These mathematical functions are performed on information stored in code and/or data memory module 2404, producing results that are stored in an activation memory module 2408.

Executable code (e.g., graph code) can load weights and other parameter information into computing hardware 2406. Code and/or data memory module 2404 may be used during forward propagation of input data and/or weight parameters during inference, producing results that may include intermediate values or activations (e.g., one or more output values from a layer or neuron of the trained neural network) stored in activation memory module 2408.

Code and/or data memory module 2404 and activation memory module 2408 may be implemented as separate memory structures, as a combined memory structure, or as partially combined and partially separate structures. Any portion of code and/or data memory module 2404 and/or activation memory module 2408 may comprise other on-chip or off-chip memory, such as a processor's L1, L2, or L3 cache, or system memory. Any portion of these modules may be internal or external to one or more processors or other hardware logic devices or circuits.

Code and/or data memory module 2404 and/or activation memory module 2408 may be implemented using cache memory, dynamic random access memory (DRAM), static random access memory (SRAM), non-volatile memory (e.g., flash memory), high-bandwidth memory (HBM), double-data-rate (DDR) RAM, single-data-rate (SDR) RAM, or other storage technologies. The choice of whether each module (e.g., each code and/or data memory module 2410 and each activation memory module 2408) is internal or external to a processor (e.g., DRAM vs. SRAM vs. flash vs. HBM vs. DDR RAM vs. SDR RAM) may depend on various factors, including available on-chip versus off-chip storage, the latency requirements of inference functions, batch size, and other performance constraints. Code and/or data memory module 2404 and/or activation memory module 2408 may also be associated with larger off-chip storage such as DRAM, NVMe devices, solid-state drives, or disk-based storage to support low-latency inference at scale.

Computing hardware 2406 may comprise one or more processors or other hardware logic devices or circuits. A processor may include one or more of the following: central processing units (CPUs), graphics processing units (GPUs), fixed-function units, or other specialized units. Code and/or data memory module 2404 and activation memory module 2408 may share a processor or other hardware logic device or circuit; alternatively, they may be associated with different processors or hardware logic devices, or with a combination of shared and distinct processors and logic resources.

In some cases, computing hardware 2406 comprises one or more processors, wherein each processor may include at least one of: an application-specific integrated circuit (ASIC), such as a tensor processing unit (TPU), a neural processing unit (NPU), an inference processing unit (IPU), or other domain-specific processing units. Each processor of computing hardware 2406 may include CPU hardware, GPU hardware, or other hardware such as field-programmable gate arrays (FPGAs), in-memory-compute (IMC) hardware, or other domain-specific hardware. In some cases, computing hardware 2406 is implemented as a system-on-a-chip (SoC).

Inference hardware structure 2402 may be used in conjunction with ASICs (such as TPUs or NPUs), IPUs, or other domain-specific processing units. In some cases, inference hardware structure 2402 is used in conjunction with CPU hardware, GPU hardware, or other hardware such as FPGAs or IMC hardware. In other cases, inference hardware structure 2402 itself may be implemented as a SoC.

Inference hardware structure 2402 may further include hardware logic that directs the use of computational resources in conjunction with weight values, other parameters, input/output data, and other information corresponding to one or more layers of neurons within a trained neural network. In one implementation, inference hardware structure 2402 includes a single memory-module/compute pairing (e.g., code and/or data memory module 2404 paired with computing hardware 2406). In other implementation, inference hardware structure 2402 includes multiple memory-module/compute pairings, with each pairing producing results that are stored in activation memory module 2408.

Inference hardware structure 2402 may further include one or more interconnect interfaces to receive input data, send output data, or transmit data between hardware components (e.g., within a node/server or across racks of servers). Such interconnects may include, for example, PCIe, CXL, NVLink, Ethernet, UALink, or others. These interconnects may carry command signals, dataflow control signals, and memory-transfer operations that regulate the order and timing of inference operations.

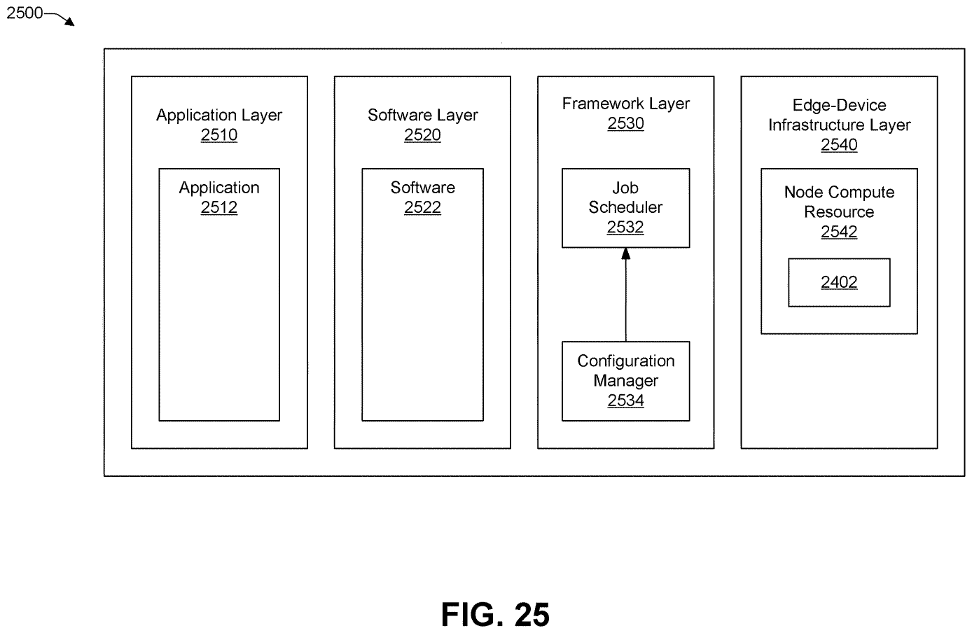

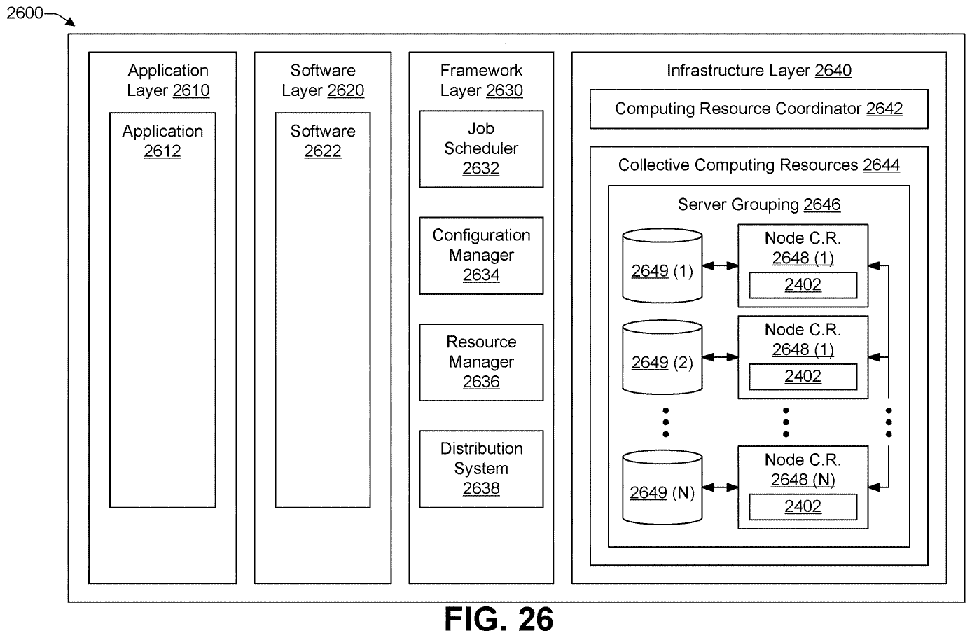

Inference hardware structure 2402 may operate as a standalone component, for example in an edge-device system as shown in FIG. 25, or may be integrated within a larger computing system, cluster, or data-center environment, as depicted in FIG. 26. In such standalone and/or integrated environments, one or more computing hardware 2406 structures may form a portion of a node computing resource within an infrastructure layer, while software and framework layers coordinate loading of model weights and parameters, dispatch inference workloads, and retrieve predicted outputs. In some cases, the ML model 404 of the present disclosure is deployed on inference hardware structure 2402. Because the compressed reasoning trace 406 has the compressed token count 606 substantially less than the natural language token count 612 of the natural language reasoning trace 604, the ML model 404 requires fewer token-generation steps per inference call, reducing the number of memory reads from code and/or data memory module 2404 and the number of compute operations performed by computing hardware 2406 per input query 402. The following sections provide further detail regarding inference hardware within standalone and integrated environments.

Edge-Device Inference System with Inference Hardware

In some cases, the ML model 404 of the present disclosure is deployed within edge device inference system 2500 of the type described with reference to FIG. 25. Edge deployment imposes strict memory capacity and inference latency constraints that conventional natural language reasoning models cannot satisfy due to their high per-query token counts. The compressed token count 606 of the compressed reasoning trace 406 reduces memory occupancy in code and/or data memory module 2404 and activation memory module 2408 and reduces the number of compute operations required of computing hardware 2406 per query, allowing the ML model 404 to generate accurate reasoning outputs at the edge without cloud offloading.

As shown in FIG. 25, an edge-device inference system 2500 is an example of an integrated system for performing inference using a trained model, such as a trained neural network. Edge device inference system 2500 may ingest data at the edge, providing real-time output data; low latency in such an environment can significantly improve inference performance. In some cases, the ML model 404 of the present disclosure is deployed within edge device inference system 2500. The compressed token count 606 of the compressed reasoning trace 406 reduces the memory occupancy and per-query inference latency of the ML model 404 to levels compatible with the resource constraints of edge deployment, enabling real-time generation of accurate reasoning outputs at the edge without cloud offloading.

FIG. 25 illustrates an exemplary edge device inference system 2500. Edge device inference system 2500 can include an application layer 2510, a software layer 2520, a framework layer 2530, and an edge-device infrastructure layer 2540. These layers cooperate to execute inference operations using a trained neural network with fixed, known weight parameters.

Edge-device infrastructure layer 2540 may include a node compute resource 2542. Node compute resource 2542 comprises inference hardware structure 2405 and may be implemented as a SoC. Node compute resource 2542 may include, in addition to the one or more processors of inference hardware structure 2402, at least one CPU or other processors (including accelerators such as ASICs, GPUs, FPGAs, NPUs, etc.), memory (e.g., DRAM, HBM, flash, DDR RAM, SDR RAM, etc.), network input/output devices (e.g., network interface cards (NICs)), network switches, power modules, cooling modules, and other supporting components.

Framework layer 2530 may include a job scheduler 2532 and a configuration manager 2534. Framework layer 2530 provides a framework to support software 2522 in software layer 2520 and one or more applications 2512 in application layer 2510. Framework layer 2530 can implement a runtime coordination system that adjusts inference execution based on the physical constraints of edge device system 2500. Job scheduler 2532 may assign inference tasks to available compute units in node compute resource 2542, while configuration manager 2534 may configure software layer 2520 and framework layer 2530, load trained model files, configure memory buffers, and establish runtime properties such as quantization settings.

Software 2522 in software layer 2520 may include an operating system, device drivers, local data stores, and service processes configured to receive inference requests, pre-process input data, interface with applications 2512 in application layer 2510 (e.g., machine learning applications), and return predicted outputs.

Applications 2512 in application layer 2510 may include one or more types of applications used by portions of node compute resource 2542. These applications may include machine-learning applications, inference engines, and machine-learning framework software (e.g., PyTorch, TensorFlow) as well as domain-specific applications that rely on inference services.

Inference logic corresponding to inference hardware structure 2402 is used to perform inference operations as described herein in conjunction with FIG. 24. In particular, inference logic within inference hardware structure 2402 can be used in edge device inference system 2500 for operations based, at least in part, on weight parameters of a trained neural network. Edge device inference system 2500 may include tools, services, software, or other resources to predict or infer information using one or more trained machine-learning models. In some cases, one or more such trained machine-learning models is implemented as the ML model 404 of the present disclosure, which generates the compressed reasoning trace 406 in response to each input query 402 received at application layer 2510, with the compressed token count 606 of the compressed reasoning trace 406 reducing the memory and compute demand placed on node compute resource 2542 per inference call.

Data Center Inference System with Inference Hardware

FIG. 26 illustrates a data center 2600 that may include an application layer 2610, a software layer 2620, a framework layer 2630, and an infrastructure layer 2640. Data center 2600 may execute inference operations such as forward-propagation computations to generate predicted outputs from input data, while providing increased efficiency, scalability, and reduced latency.

Data center 2600 can be organized into a multilayer architecture in which infrastructure layer 2640 comprises a computing resource coordinator 2642, collective computing resources 2644, server grouping 2646, and a plurality of node computing resources (node C.R.s) 2648(1)-2648(N), where N is a positive integer. Each node C.R. 2648(i) may include one or more CPUs or other processors (e.g., accelerators such as ASICs, GPUs, FPGAs, etc.), memory storage devices 2649(1)-2649(N) (e.g., DRAM, NVMe, solid-state drives (SSDs), or disk-based storage devices), network input/output components, network switches, virtual-machine hosts, power regulation modules, and cooling assemblies. In some cases, each node C.R. corresponds to a complete physical or virtual server.

Collective computing resources 2644 may include at least one server grouping 2646, representing a logical grouping of node C.R.s such as one or more server racks. Server grouping 2646 may be deployed in a single data-center location (non-distributed) or geographically distributed. Server grouping 2646 and/or collective computing resources 2644 may pool compute, network, or memory resources to process inference workloads.

Computing resource coordinator 2642 configures, allocates, manages, and coordinates each node C.R. and resources within server grouping 2646 and/or collective computing resources 2644. Computing resource coordinator 2642 (e.g., Kubernetes, Slurm, or other resource managers) may coordinate hardware and software resources, such as compute, storage, and network capabilities of infrastructure layer 2640. Computing resource coordinator 2642 may perform software-defined infrastructure control operations to allocate inference workloads in data center 2600.

Framework layer 2630 may include a job scheduler 2632, a configuration manager 2634, a resource manager 2636, and a distribution system 2638. Framework layer 2630 supports software 2622 in software layer 2620 and one or more applications 2612 in application layer 2610. For example, framework layer 2630 may receive inference queries or pre-processed input data from software layer 2620 or application layer 2610 and return predicted outputs for post-processing and delivery to users or client systems.

Job scheduler 2632 may be implemented using a distributed processing framework, such as Apache Spark™, and can leverage distribution system 2638 for large-scale data processing. Distribution system 2638 may provide input data, model files, and other assets needed for inference workloads. Job scheduler 2632 coordinates workloads across the layers of data center 2600. Configuration manager 2634 may configure software layer 2620 and framework layer 2630 (including Spark™ and distribution system 2638), generate configurations specifying model versions, precision formats, and hardware requirements, and provide such configurations to resource manager 2636. Resource manager 2636, in conjunction with computing resource coordinator 2642, identifies suitable node C.R.s 2648(1)-2648(N) for each workload, after which job scheduler 2632 dispatches the workloads to the selected nodes.

Software 2622 in software layer 2620 may include operating systems, database software, model-serving software, data-preparation software, and other components supporting inference operations and integration with applications.

Applications 2612 in application layer 2610 may be used by one or more node C.R.s 2648(1)-2648(N), collective computing resources 2644, and/or distribution system 2638. Applications 2612 may include machine-learning applications, analytics services, or other applications that rely on inference pipelines.

Trained machine-learning models corresponding to one or more trained neural networks can be used within data center 2600 to infer or predict information at scale. In some cases, one or more such trained machine-learning models is implemented as the ML model 404 of the present disclosure. Because each inference call of the ML model 404 generates the compressed reasoning trace 406 with a compressed token count 606 substantially less than the natural language token count 612 of the natural language reasoning trace 604, each inference workload dispatched by job scheduler 2632 to node C.R.s 2648(1) through 2648(N) requires fewer token-generation steps, reducing memory bandwidth, compute cycles, and interconnect traffic per response at scale within data center 2600.

The following description illustrates dataflow through data center 2600. This dataflow includes receipt of user input or pre-processed data, task configuration and scheduling within framework layer 2630, and execution of inference on node C.R.s 2648(1)-2648(N) to return predicted outputs. This discussion demonstrates how the layered architecture of data center 2600 supports efficient processing, parallelism, and low-latency delivery of inference results.

### Example: Dataflow Through Data Center Inference Hardware

Dataflow through data center 2600 may begin when an inference request is received at application layer 2610, for example as a data stream of user prompts or as data from an edge device. Received text may be stored in system memory (e.g., DRAM). Software layer 2620 may perform tokenization or other pre-processing operations on a host CPU, prepare one or more inference workloads corresponding to the tokenized inputs, and send these workloads to framework layer 2630 for scheduling and execution.

Framework layer 2630 coordinates distribution of the inference workloads. Job scheduler 2632 may consult resource manager 2636 to determine which node C.R.s 2648(1)-2648(N) have sufficient available compute and memory to execute the workloads efficiently. Resource manager 2636, in conjunction with computing resource coordinator 2642, identifies suitable node C.R.s. Model files, weight tensors, and other runtime data may be loaded from distribution system 2638 (e.g., NVMe or a distributed filesystem) into DRAM and then into accelerator memory such as HBM.

Execution of inference workloads occurs within the allocated node C.R.s using one or more instances of inference hardware structure 2402. Inference results, including intermediate activations or final predicted outputs, may be returned from node C.R.s to job scheduler 2632, which can aggregate outputs when multiple nodes are involved. In some cases in which the ML model 404 of the present disclosure executes within node C.R.s 2648(1) through 2648(N), each inference workload produces the compressed reasoning trace 406 comprising the sequence of symbolic reasoning steps 408. The final answer 420 extracted from the answer expression 418 corresponding to the answer operator 416 of the compressed reasoning trace 406 constitutes the predicted output returned to job scheduler 2632. Framework layer 2630 then returns predicted outputs to software layer 2620 and/or application layer 2610 for post-processing, formatting, or delivery. The layered architecture of data center 2600 thus supports coordinated data movement and scalable execution of inference workloads, with the compressed token count 606 of the compressed reasoning trace 406 reducing the per-response resource footprint at each node C.R.

In some cases, the ML model 404 of the present disclosure is deployed within data center 2600 of the type described with reference to FIG. 26, executing inference workloads across node C.R.s 2648(1) through 2648(N). Because the compressed reasoning trace 406 has a compressed token count 606 substantially less than the natural language token count 612 of the natural language reasoning trace 604, each inference workload generated by the ML model 404 requires fewer tokens per response. This reduction in per-response token count decreases the memory bandwidth, compute cycles, and interconnect traffic associated with each inference workload, allowing data center 2600 to serve more input queries 402 per unit time and reducing the energy cost per response at scale.

### MCP Architecture

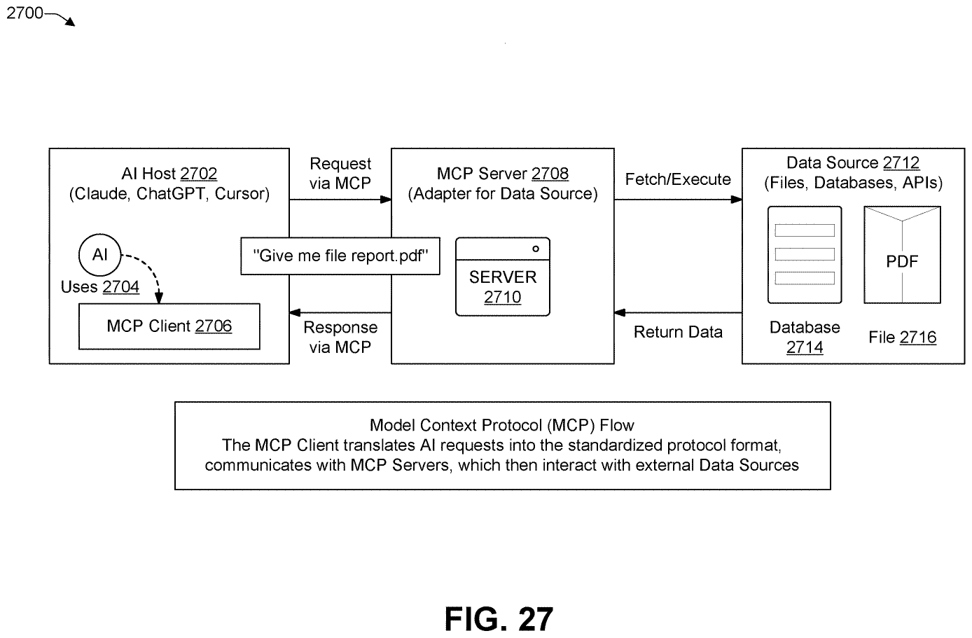

FIG. 27 is a system architecture diagram illustrating the Model Context Protocol (MCP) architecture 2700 and data flow between core components for processing AI requests through standardized protocol communication and interaction with external data sources.

The Model Context Protocol addresses the challenge of enabling AI applications to access external data sources, tools, and services in a standardized, secure, and interoperable manner. Traditional AI systems often require custom integration code for each external service, resulting in fragmented implementations and significant maintenance burdens. The MCP architecture solves this problem by introducing a universal protocol that acts as a standardized communication layer between AI applications and external systems, enabling any AI application to communicate with any MCP-compatible server without implementing bespoke integration code for each pairing.

The MCP architecture 2700 comprises three core components: an MCP host, an MCP client, and an MCP server, which collaborate to facilitate seamless communication between AI applications, external tools, and data sources. The architecture embodies (i) standardization, via a protocol specification defining message formats and communication patterns; (ii) modularity, via separation between AI logic and data-access logic; (iii) extensibility, via the ability to add new capabilities without modifying existing components; and (iv) security, via built-in authentication and authorization mechanisms.

The AI Host 2702 represents an AI application that provides the environment for executing AI-based tasks while running the MCP client. Examples include Claude Desktop (for AI-assisted content creation), ChatGPT (for conversational AI), and Cursor (for AI-powered integrated development and code-completion). AI Host 2702 integrates interactive tools and data to enable smooth communication with external services, hosts the MCP client, and ensures communication with external MCP servers.

Within AI Host 2702, an AI component 2704 represents the core artificial intelligence model or reasoning engine that generates requests, processes information, and produces outputs. In some cases, AI component 2704 is implemented as the ML model 404 of the present disclosure. AI component 2704 is operatively connected to MCP Client 2706, indicating that the AI component uses MCP Client 2706 to communicate with external systems. This connection enables AI component 2704 to access external functionalities and data sources, thereby augmenting its inherent knowledge with real-time information, user-specific data, and external computational resources. When implemented as the ML model 404, AI component 2704 incorporates retrieved context into the input query 402 and generates the compressed reasoning trace 406, with the compressed token count 606 of the compressed reasoning trace 406 reducing the inference latency of AI component 2704 within the MCP request-response cycle.

MCP Client 2706 operates within the host environment as the protocol implementation layer. MCP Client 2706 acts as an intermediary component, maintaining a one-to-one communication link with a corresponding MCP Server 2708. Operating on behalf of the host, MCP Client 2706 initiates requests to MCP Server 2708, queries available functions, and retrieves responses describing the server's tools, resources, and capabilities. This design allows the host to access and use external functionalities provided by MCP servers 2708 in a uniform manner.

MCP Client 2706 performs request translation by converting high-level AI requests into standardized MCP protocol messages with defined schemas and data types. MCP Client 2706 also performs capability discovery by querying MCP Server 2708 to determine available tools, resources, and prompts. MCP Client 2706 manages connection lifecycle operations, including establishing secure connections, maintaining connection state, implementing authentication flows, and handling errors. MCP Client 2706 processes notifications from MCP servers 2708, providing real-time updates about task progress and system status, and performs sampling to gather data on tool usage and performance to support optimization and informed decision-making. MCP Client 2706 communicates with MCP Servers 2708 via a transport layer that facilitates secure, reliable data exchange.

A request via the MCP communication pathway extends from AI Host 2702 to MCP Server 2708, indicating transmission of requests using the Model Context Protocol. Communication begins with an initial request from MCP Client 2706 querying MCP Server 2708 regarding available functions, tools, resources, and capabilities. The protocol defines a structured message format including a message identifier, operation type, parameters, authentication information, and metadata.

An exemplary request, “Give me file report.pdf,” demonstrates a use case in which AI component 2704 requests access to a specific file resource. MCP Client 2706 translates this high-level request into a structured MCP protocol message specifying a resource-retrieval operation, identifying the target resource, including necessary parameters, and providing authentication credentials.

MCP Server 2708 enables the MCP host and client to access external systems and execute operations, functioning as an adapter that mediates access to external data sources. MCP servers 2708 implement an adapter pattern that encapsulates the logic required to interact with specific data sources, translating standardized MCP protocol operations into native operations required by those systems. As a result, adding support for new data sources typically requires only the implementation of a new MCP server 2708, without changes to AI applications or MCP clients 2706.

MCP Server 2708 provides three core capabilities: tools, resources, and prompts. Tools allow MCP Server 2708 to invoke external services and APIs to execute operations on behalf of AI models. When a host application needs to perform an operation, it first queries MCP Server 2708 via MCP Client 2706 to obtain a list of available tools and their capabilities. Based on task context, the host selects an appropriate tool and issues an invocation request via MCP Client 2706. MCP Server 2708 then executes the corresponding operation through an external service or API and returns the result to MCP Client 2706, which forwards the result to the host. For example, if an AI model requires real-time weather data, the host identifies a weather tool from the server's advertised capabilities, MCP Server 2708 performs the API call, and the resulting data is returned to the host.

Unlike traditional function-calling interfaces confined within a single model or framework, MCP tools are described and accessed through a standardized, model-agnostic protocol. This design allows tools to be dynamically discovered, described, and invoked across heterogeneous AI systems, ensuring cross-platform interoperability. MCP tools support bidirectional communication between a host and a service, enabling richer interactions such as asynchronous updates or event streaming. Once configured, these tools follow a supply-and-consume model that promotes modularity and reusability, allowing independently developed tools to interoperate seamlessly and improving system efficiency and extensibility.

Resources provide access to structured and unstructured datasets that MCP Server 2708 can expose to AI models. These datasets may reside in local storage, databases, or cloud platforms. When an AI model requests specific data, MCP Server 2708 retrieves and processes the relevant information, enabling the model to make data-driven decisions. For example, a recommendation system may access customer interaction logs, while a document summarization task may query a text repository. In the “report.pdf” example, MCP Server 2708 uses its resources capability to access the requested file from available sources.

Prompts are predefined templates and workflows that MCP Server 2708 generates and maintains to optimize AI responses and streamline repetitive tasks. They provide consistency in responses and improve task execution efficiency. For example, a customer-support chatbot may use prompt templates to ensure uniform and accurate responses, while an annotation workflow may rely on predefined prompts to maintain consistency in labeling across multiple annotators.

A response via the MCP communication pathway extends from MCP Server 2708 back to AI Host 2702, indicating transmission of responses using the Model Context Protocol. Upon receiving a request, MCP Server 2708 responds with an initial response listing available tools, resources, and prompts that MCP Client 2706 can use. The response message includes a message identifier, status indicator, response payload, and metadata. MCP Client 2706 processes the response and provides it to AI component 2704 within AI Host 2702.

A fetch/execute communication pathway extends from MCP Server 2708 to a Data Source 2712, indicating that MCP Server 2708 invokes external APIs or otherwise executes operations to retrieve and process information from external data sources. MCP Server 2708 may invoke external APIs using whatever protocols, authentication mechanisms, and access methods are required by those data sources. This isolation of data-source-specific details within MCP Server 2708 ensures that MCP Client 2706 and AI Host 2702 remain agnostic to whether data is sourced from SQL databases, REST APIs, file systems, or other sources.

Data Source component 2712 represents external systems and data repositories that MCP Server 2708 can access to fulfill client requests. Data Source 2712 can include three primary categories: files 2716, databases 2714, and APIs. Files 2716 represent file-system resources and local storage containing documents such as PDF files, plain-text files, structured data files, and binary files. Databases 2714 represent database systems that store structured and unstructured data, providing persistent data storage that MCP Server 2708 can query to retrieve information for AI models. APIs represent Internet-based services, web APIs, cloud platforms, and remote computational resources accessible via network protocols.

A return data communication pathway extends from Data Source 2712 back to MCP Server 2708, indicating that retrieved data or operation results are returned from external data sources to MCP Server 2708 for processing and subsequent delivery to MCP Client 2706. This bidirectional data flow shows that MCP Servers 2708 invoke APIs or execute fetch operations to access external data sources and then process and return results through the MCP workflow back to AI Host 2702.

The MCP flow can be summarized as: “The MCP Client translates AI requests into standardized protocol format, communicates with MCP Servers, which then interact with external Data Sources.” This captures the essential data flow and transformation in MCP architecture 2700. Translation from AI requests to standardized protocol messages bridges the gap between how AI systems express needs and how protocols structure messages. Communication between MCP Clients 2706 and MCP Servers 2708 occurs via the transport layer according to the protocol specification, enabling interoperability between independently developed components. Interactions between MCP Servers 2708 and external Data Sources 2712 encapsulate the complexity and diversity of backend systems, presenting a uniform interface to clients.

The complete workflow proceeds as follows: AI component 2704 generates a request based on user input or autonomous operation; MCP Client 2706 translates the request into standardized protocol format; MCP Client 2706 transmits the request via MCP to MCP Server 2708, querying available capabilities and requesting operations or data; MCP Server 2708 determines which capabilities are needed; MCP Server 2708 executes a fetch/execute operation to interact with appropriate Data Source 2712; Data Source 2712 processes the operation and returns requested data to MCP Server 2708; MCP Server 2708 processes the returned data and formulates a response; MCP Server 2708 transmits the response via MCP back to AI Host 2702; MCP Client 2706 processes the response and provides it to AI component 2704; and AI component 2704 uses the data or results to complete its task.

For the exemplary request “Give me file report.pdf,” AI component 2704 generates a retrieval request; MCP Client 2706 translates it into standardized MCP format specifying a resource retrieval; MCP Server 2708 receives the request and identifies its resources capability; MCP Server 2708 performs a fetch operation targeting file data source 2716; file data source 2716 locates and returns file data to MCP Server 2708; MCP Server 2708 formulates a response containing the PDF or a reference thereto; MCP Server 2708 transmits the response back to AI Host 2702; and MCP Client 2706 delivers the file data to AI component 2704 for display or further processing.

This architecture demonstrates a modular and extensible system in which MCP Client 2706 acts as an intermediary that translates AI requests into standardized protocol messages, maintains a one-to-one communication link with its corresponding MCP Server 2708, and enables seamless integration between AI applications and diverse external systems (including files 2716, databases 2714, and APIs within Data Source 2712). In some cases, in which AI component 2704 is implemented as the ML model 404 of the present disclosure, the reduced inference latency resulting from the compressed token count 606 of the compressed reasoning trace 406 shortens the time from initial MCP request to generation of the final answer 420, improving the end-to-end throughput of MCP architecture 2700. The standardized protocol format ensures cross-platform interoperability, allowing different AI hosts 2702 to communicate with various MCP Servers 2708 and access heterogeneous data sources 2712 via a unified interface. The adapter architecture of MCP Server 2708 provides a consistent abstraction layer that shields AI Hosts 2702 from implementation details of different data sources, enabling AI applications to access files 2716, databases 2714, and APIs through a single standardized protocol without requiring custom integration code for each data source type.

In some cases, the ML model 404 of the present disclosure is implemented as AI component 2704 within AI Host 2702 of MCP architecture 2700. In that configuration, AI component 2704 receives context retrieved by MCP Client 2706 from MCP Server 2708 and incorporates that context into the input query 402 passed to the ML model 404. The ML model 404 generates the compressed reasoning trace 406, comprising the sequence of symbolic reasoning steps 408 without natural language words. The reduced token count of the compressed reasoning trace 406 reduces the inference latency of AI component 2704, enabling AI Host 2702 to complete its reasoning and issue follow-up MCP requests more rapidly, improving end-to-end throughput of MCP architecture 2700.

### MCP Workflow

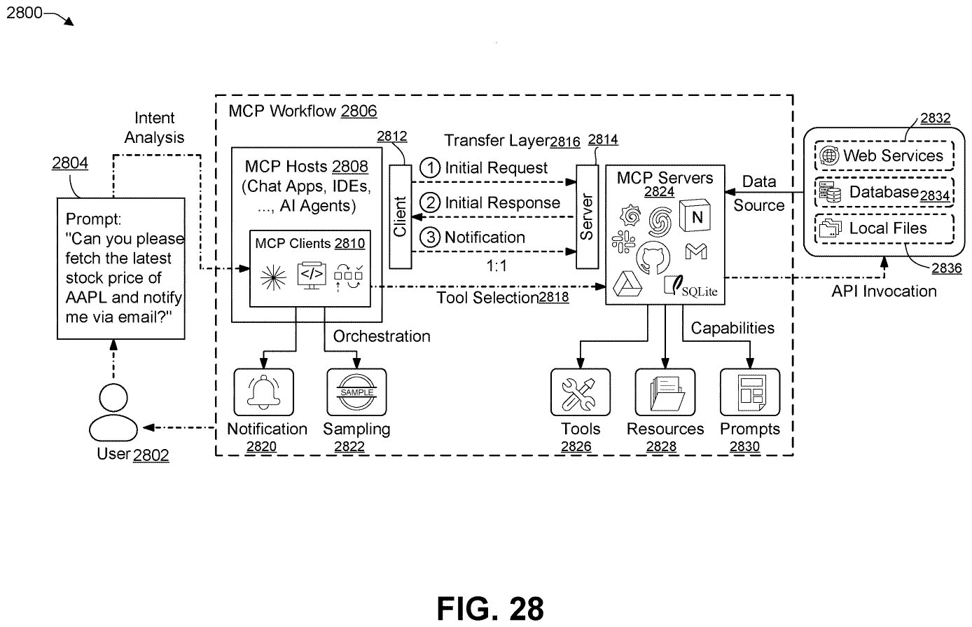

FIG. 28 is a detailed system architecture diagram illustrating a complete Model Context Protocol (MCP) workflow 2800 and the comprehensive interaction among core components, subcomponents, communication pathways, and external systems for processing user requests through multiple stages, including intent analysis, tool selection, and API invocation.

The system architecture comprises a user entity 2802 operatively connected via a communication pathway to a prompt input interface 2804. User entity 2802 initiates requests and receives notifications from MCP workflow system 2806.

Prompt input interface 2804 presents an exemplary user query: “Prompt: ‘Can you please fetch the latest stock price of AAPL and notify me via email?”’ This example illustrates a typical use case in which the user requests real-time financial market data for Apple Inc. (ticker AAPL) from external data sources and also requests automatic delivery of the results via email. The example demonstrates that the system can process complex, multi-step requests requiring both data retrieval and subsequent communication actions. Prompt input interface 2804 is operatively connected to MCP workflow system 2806, indicating the initial direction of information flow from the user into the MCP workflow.

MCP workflow system 2806 comprises the three core components of the MCP architecture: MCP host, MCP client, and MCP server, which jointly enable secure, well-managed communication between AI applications, external tools, and data sources. MCP hosts 2808 (labeled “Chat Apps, IDEs, . . . , AI Agents”) indicate non-limiting examples of applications functioning as MCP hosts. Examples include Claude Desktop, Cursor, and autonomous AI agents. In some cases, the AI component executing within MCP host 2808 is implemented as the ML model 404 of the present disclosure, receiving intent-analyzed and MCP-enriched context as the input query 402 and generating the compressed reasoning trace 406 comprising the sequence of symbolic reasoning steps 408. MCP host 2808 provides the environment for executing AI tasks while running the MCP client; it integrates interactive tools and data to enable communication with external services and establishes the runtime environment required for MCP client operations.

In the host-client-server architecture, the MCP client acts as an intermediary, maintaining a one-to-one communication link with a corresponding MCP server. The MCP client initiates requests, queries available functions, and retrieves responses describing tools, resources, and capabilities, while also processing notifications and performing sampling for optimization purposes. MCP client 2812 communicates with MCP servers 2824 via transport layer 2816, ensuring secure, reliable data exchange between host 2808 and external resources.

An Intent Analysis component indicates that MCP client 2812 analyzes the intent of user prompts. When user prompt 2804 is sent to MCP client 2812, MCP client 2812 interprets semantic content, contextual meaning, and operational requirements to determine the user's intent and required system actions (e.g., retrieve stock data and send email).

An Orchestration component indicates coordination functions performed by MCP client 2812. Orchestration governs sequencing and management of operations as MCP client 2812 interacts with MCP servers 2824 and manages workflow from initial request through final notification.

A Notification feature 2820 indicates notification functionality connected back to user entity 2802. Notifications include task completion confirmations, progress updates, error messages, and result payloads. MCP client 2812 also performs sampling 2822, collecting data about tool usage and performance to support system optimization and resource planning.

Transport layer 2816 facilitates bidirectional data exchange between clients 2812 and servers 2814, managing transmission of initial requests, server responses describing capabilities, and notifications. Within transport layer 2816, communication between MCP client 2812 and MCP server 2814 follows three stages:

Initial Request: Communication flows from MCP client 2812 to MCP server 2814. MCP client 2812 sends an initial request querying MCP server 2814 for available functions, tools, resources, and capabilities. In the AAPL example, the initial request seeks confirmation of available stock-price tools and email-notification tools.

Initial Response: Communication flows from MCP server 2814 back to MCP client 2812. MCP server 2814 responds with an initial response listing available tools, resources, and prompts that MCP client 2812 can use for task execution, such as tools for retrieving stock quotes and sending email notifications.

Notification: A bidirectional notification channel enables ongoing updates between MCP servers 2814 and MCP clients 2812. Once a connection is established, the system maintains a continuous exchange of notifications 2820, ensuring that changes in server status and progress updates are communicated to MCP client 2812 in real time.

The notation “1:1” between “Client” 2812 and “Server” 2814 indicates a one-to-one communication link between an MCP client and its corresponding MCP server during request and response exchange, establishing a dedicated communication relationship for each client-server pair.

A Tool Selection component 2818 within transport layer 2816 indicates tool selection functionality. Based on task context and intent analysis, host 2808 selects appropriate tools from those available on MCP server 2824 and issues invocation requests via MCP client 2812. For the AAPL example, tool selection 2818 would select (i) a financial-data tool capable of accepting a ticker symbol and returning current price information, and (ii) an email-service tool capable of composing and delivering email messages.

MCP servers 2824 are depicted along with icons representing various service integrations (e.g., GitHub, email/messaging, Google Drive, code repositories, SQLite). MCP servers 2824 provide three core capabilities: tools 2826, resources 2828, and prompts 2830. Tools 2826 enable MCP servers 2824 to invoke external services and APIs and return results through a standardized, model-agnostic protocol; resources 2828 provide access to structured and unstructured datasets; and prompts 2830 define templates and workflows to standardize and optimize AI responses.

External data sources include web services 2832 (representing Internet-based services, APIs, cloud platforms, and remote computation), databases 2834 (representing structured and unstructured data repositories), and local files. API invocation 2836 indicates that these data sources are accessed via APIs. MCP server 2824 invokes external APIs, retrieves results, and returns processed data through MCP workflow system 2806.

The complete workflow proceeds as follows: user 2802 sends prompt 2804; MCP client 2812 performs intent analysis; MCP client 2812 selects tools 2826 via MCP server 2824; MCP server 2824 invokes external APIs 2836 to retrieve and process information from web services 2832, databases 2834, and local files; and MCP workflow system 2806 notifies user 2802 of results via notification pathway 2820. Communication follows the structured process of initial request, initial response, and ongoing notifications, enabling high-performance, synchronized interactions with external resources.

For the exemplary prompt “Can you please fetch the latest stock price of AAPL and notify me via email?”, user 2802 submits prompt 2804 to MCP client 2812. MCP client 2812 identifies a need for financial-data retrieval and email notification, sends an initial request via transport layer 2816 to MCP server 2814, receives an initial response enumerating available financial and email tools, and coordinates selection of appropriate tools 2826. MCP server 2824 then invokes a financial-data API 2836 with ticker “AAPL,” receives current stock price information from web services 2832, returns this data to MCP client 2812, and subsequently invokes an email-service API 2836 to compose and deliver a notification message containing the stock price to the user's email. Notifications 2820 keep user 2802 informed throughout. A one-to-one communication link between MCP client 2812 and MCP server 2814 provides dedicated, reliable communication, while sampling 2822 gathers performance and usage data for optimization.

This architecture demonstrates a modular, extensible system in which MCP client 2812 maintains a one-to-one link with MCP server 2814 and enables seamless integration between AI applications 2808 and diverse external systems including web services 2832, databases 2834, and local files, while ensuring secure, reliable data exchange through transport layer 2816 throughout workflow 2800, from initial prompt 2804 to final notification 2820. In some cases, in which the AI component within MCP host 2808 is implemented as the ML model 404 of the present disclosure, the compressed token count 606 of the compressed reasoning trace 406 reduces the time spent in the generation step of workflow 2800, shortening end-to-end latency from intent analysis through delivery of notification 2820 to user 2802.

### Artificial Intelligence System Architecture

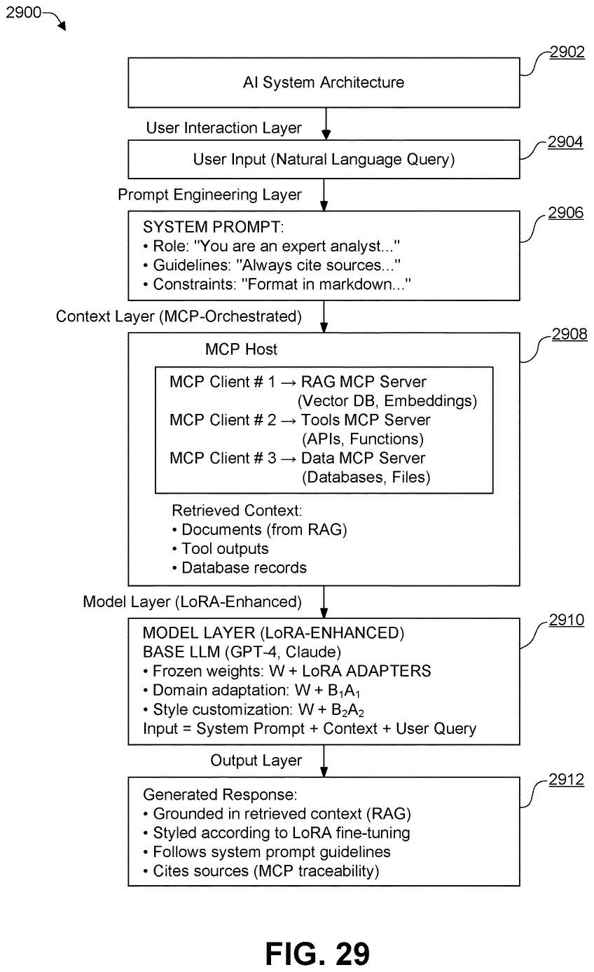

FIG. 29 illustrates a comprehensive artificial intelligence system architecture 2900 that integrates multiple technological components to enhance language-model performance. The system comprises five principal layers arranged in sequential data flow: a User Interaction Layer, a Prompt Engineering Layer, a Context Layer implementing the Model Context Protocol, a Model Layer enhanced with Low-Rank Adaptation, and an Output Layer.

At the uppermost portion of FIG. 29, User Interaction Layer 2904 receives user input in the form of natural-language queries. Such queries comprise unstructured textual input formulated in human-readable language and serve as the initiating stimulus for subsequent processing. Queries may include interrogative statements, imperative commands, or declarative sentences expressing an informational need or task requirement.

The natural-language query is transmitted from User Interaction Layer 2904 to Prompt Engineering Layer 2906. Prompt Engineering Layer 2906 comprises a system-prompt component that defines operational parameters and behavioral constraints for the language model. The system prompt includes a role definition specifying the persona or expertise domain of the AI agent (e.g., “You are an expert analyst”), guidelines that establish mandatory behaviors (e.g., “Always cite sources”), and constraints specifying output formatting and structure (e.g., “Format in markdown”). This system prompt establishes a persistent behavioral framework that governs response generation across a session or task, providing consistent operational parameters independent of the specific content of individual queries.

The processed query, structured in accordance with system-prompt specifications, is transmitted to Context Layer 2908, labeled “Context Layer (MCP-Orchestrated).” Context Layer 2908 implements the Model Context Protocol and includes an MCP Host application that orchestrates access to external data sources and tools. The MCP Host coordinates and manages multiple MCP client connections, each maintaining a dedicated one-to-one connection with a corresponding MCP server as specified by the MCP architecture.

Within the MCP Host, three MCP clients are illustrated. A first MCP client is connected to a RAG MCP Server that provides access to vector-database storage and embedding generation. The RAG MCP Server performs semantic similarity search by converting textual queries into dense vectors via embedding models, comparing those vectors to pre-indexed document vectors in a vector database, and retrieving documents with high similarity scores. These documents supply contextual information that augments the language model input.

A second MCP client connects to a Tools MCP Server that exposes executable functions extending the language model's capabilities beyond pure text generation. The Tools MCP Server provides access to APIs and computational functions that enable the language model to perform tasks such as mathematical calculations, data transformations, external service invocations, and other programmatic operations. Tools are discoverable via the MCP tool-listing mechanism and invocable via standardized JSON-RPC message exchanges.

A third MCP client connects to a Data MCP Server providing access to structured and unstructured data sources such as relational databases, document stores, and file systems. The Data MCP Server executes queries and retrieves file contents, providing the language model with up-to-date operational data not necessarily present in its pre-training corpus.

Context Layer 2908 aggregates retrieved context from the RAG, Tools, and Data MCP servers. The aggregated context comprises RAG-retrieved documents, tool outputs, and database records or file contents, as explicitly enumerated in FIG. 29. This retrieved context is structured and injected into the language model's input prompt, augmenting the model's pre-trained knowledge with real-time, task-specific, domain-relevant information.

The augmented prompt comprising the system prompt from Prompt Engineering Layer 2906, retrieved context from Context Layer 2908, and the user query from User Interaction Layer 2904 is transmitted to Model Layer 2910, labeled “Model Layer (LoRA-Enhanced).” In some cases, the base LLM in Model Layer 2910 is implemented as the ML model 404 of the present disclosure, trained using the multi-stage training process 904 described with reference to FIGS. 9 and 10. Model Layer 2910 includes a base LLM (e.g., GPT-4 or Claude) whose pre-trained weights are maintained in a frozen state, represented as matrix W. The frozen weights ensure that original parameters learned during pre-training remain untouched during task-specific adaptation, avoiding the computational and memory cost of full model retraining. When the base LLM is implemented as the ML model 404, the augmented prompt comprising the system prompt, retrieved context, and user query is received as the input query 402, and the ML model 404 generates the compressed reasoning trace 406 comprising the sequence of symbolic reasoning steps 408.

LoRA adapters are operatively coupled to the base LLM to implement Low-Rank Adaptation, a parameter-efficient fine-tuning technique. Each LoRA adapter includes trainable low-rank matrices that, when multiplied, approximate weight updates to the base model without altering W. The figure illustrates two exemplary LoRA adaptations, specifically domain adaptation represented mathematically as W plus B₁A₁, and style customization represented mathematically as W plus B₂A₂. In these mathematical representations, W denotes the original frozen weight matrix of the base model, while B₁, A₁, B₂, and A₂ denote low-rank matrices that are trainable during the adaptation process. The dimensions of the low-rank matrices B and A are substantially smaller than the dimensions of the base weight matrix W, such that the product of B and A provides a low-rank approximation of the weight updates that would be computed during full fine-tuning. Specifically, if the base weight matrix W has dimensions d by d, the low-rank matrices may have dimensions d by r and r by d respectively, where r is the rank hyperparameter and r is much less than d. This dimensional reduction enables parameter-efficient fine-tuning wherein the number of trainable parameters is reduced by factors of one thousand to ten thousand or more compared to full fine-tuning approaches, while maintaining model quality comparable to full fine-tuning on downstream tasks.

The Model Layer 2910 processes an input comprising the system prompt, the retrieved context, and the user query, as explicitly stated in the figure. In some cases in which the base LLM is implemented as the ML model 404 of the present disclosure, this input is received as the input query 402, and the ML model 404 generates the compressed reasoning trace 406, with the final answer 420 extracted from the answer expression 418 corresponding to the answer operator 416 transmitted to Output Layer 2912. The processing applies both the frozen base model weights W and the trainable LoRA adapter weights B₁A₁ and B₂A₂ to generate a contextually-aware, domain-adapted response. The domain adaptation component W plus B₁A₁ provides behavioral modifications specific to particular knowledge domains or task categories, while the style customization component W plus B₂A₂ provides stylistic and formatting adaptations that ensure consistency with desired output conventions. The combined effect of the frozen base weights W and the low-rank adaptations B₁A₁ and B₂A₂ produces model outputs that exhibit both the general language understanding capabilities acquired during pre-training and the task-specific behaviors learned during efficient adaptation.

The generated output is transmitted to Output Layer 2912. Output Layer 2912 produces a response exhibiting several distinguishing characteristics: (i) grounding in retrieved context, ensuring that factual content is anchored to information obtained through RAG and other MCP-mediated sources and thereby reducing hallucinations; (ii) styling according to LoRA fine-tuning, which enforces domain-specific terminology and formatting; (iii) adherence to system-prompt guidelines, ensuring compliance with role definitions, operational rules, and formatting constraints; and (iv) source citation through MCP traceability, wherein references to specific documents, database records, or tool outputs that contributed to the response are included for transparency and verifiability.

The architecture in FIG. 29 provides multiple technical advantages over conventional language-model implementations. The MCP-orchestrated Context Layer 2908 enables dynamic access to real-time, updatable information sources without retraining, allowing responses grounded in data created or updated after the model's pre-training cutoff LoRA in Model Layer 2910 enables parameter-efficient adaptation with significantly fewer trainable parameters while achieving comparable quality to full fine-tuning. Prompt Engineering Layer 2906 ensures behavioral and formatting consistency across diverse queries. The MCP protocol provides modular extensibility, allowing new tools and data sources to be added via new MCP servers without modifying the base model or existing integrations. Structured context management via MCP improves traceability and explainability by attributing responses to specific external sources, facilitating verification and auditing. The layered architecture 2900 enforces a separation of concerns, enabling independent optimization of behavioral adaptation (LoRA), knowledge access (MCP), and operational guidelines (system prompts). In some cases, in which Model Layer 2910 employs the ML model 404 of the present disclosure, architecture 2900 achieves an additional advantage: the compressed token count 606 of the compressed reasoning trace 406 reduces inference latency within Model Layer 2910 relative to the natural language model of equivalent parameter count, allowing architecture 2900 to deliver final answers 420 with lower end-to-end latency from User Interaction Layer 2904 through Output Layer 2912.

Data flow proceeds sequentially: user input is received at User Interaction Layer 2904 and passed to Prompt Engineering Layer 2906 for application of system-prompt specifications; the structured query is forwarded to Context Layer 2908, where the MCP Host coordinates retrieval of relevant context from multiple MCP servers; the augmented prompt (system prompt+context+query) is then passed to Model Layer 2910 for processing by the base LLM and LoRA adapters; and finally the generated response is output via Output Layer 2912 with grounding, styling, guideline adherence, and source attribution.

Context Layer 2908 implements communication between MCP clients and servers using JSON-RPC 2.0, a stateless, lightweight remote procedure call protocol that uses JSON for message encoding. JSON-RPC 2.0 standardizes requests, responses, and notifications exchanged between MCP clients and servers. Transport mechanisms include STDIO transport for local MCP servers (via standard input/output streams between processes on the same machine) and HTTP with Server-Sent Events for remote MCP servers (HTTP POST requests from client to server and SSE for streaming notifications and progress updates from server to client). The HTTP transport supports distributed deployments and integrates with standardized authentication mechanisms such as OAuth 2.1, wherein access tokens obtained via OAuth flows are included in HTTP headers to authenticate client requests to protected MCP servers.

System architecture 2900 thus provides a comprehensive, modular, and technically advanced implementation for AI applications requiring dynamic knowledge access, parameter-efficient adaptation, and explainable response generation. By integrating prompt engineering in Prompt Engineering Layer 2906, MCP-based retrieval in Context Layer 2908, low-rank adaptation in Model Layer 2910, and structured output in Output Layer 2912, the system produces responses that are simultaneously grounded in authoritative external sources, tailored to domain-specific requirements, consistent with operational guidelines, and traceable to verifiable information sources.

### Technical Improvements Over Traditional Machine Learning Techniques

Having described the Model Context Protocol architecture and its integration into a layered AI system, the following description addresses the technical improvements and performance enhancements that LLM-based architectures provide over traditional machine-learning techniques.

Large language models (LLMs) deliver several concrete technical improvements over traditional machine-learning architectures by enabling unified, context-aware processing pipelines that avoid the need for handcrafted feature engineering or rigid data schemas. Classical models (e.g., support vector machines (SVMs), logistic regression, shallow neural networks) typically operate on sparse or fixed-dimensional vectorizations derived from manual feature-engineering pipelines and cannot easily capture long-range dependencies or nuanced semantic relationships without extensive custom preprocessing.

In contrast, LLMs employ transformer-based attention mechanisms capable of jointly modeling heterogeneous inputs—including free-text descriptions, structured clinical data, metadata, and domain-specific features—within a single computational graph. This design supports automatic extraction of latent semantic relationships, implicit reasoning patterns, and long-range dependencies that traditional models cannot represent without significant architectural complexity.

LLMs also support zero-shot and few-shot inference, allowing new tasks and datasets to be accommodated via prompting, without retraining end-to-end pipelines. Parameter-efficient tuning methods such as LoRA and QLoRA further reduce retraining overhead by restricting updates to a small fraction of model parameters. LLM-based workflows can integrate explainability signals (e.g., attention visualizations, prompt-based reasoning traces) directly into prompts, enabling hybrid symbolic-neural reasoning that is difficult to replicate with conventional models.

Moreover, LLMs exhibit improved robustness to class imbalance, temporal domain drift, and missing structured fields by leveraging contextual embeddings instead of relying solely on brittle structured inputs. Collectively, these capabilities constitute specific technical enhancements that materially improve the functioning of underlying computing systems relative to conventional machine-learning approaches.

Specific technical improvements enabled by LLM-based architectures include:

Context-integrated feature extraction: The model dynamically derives semantic, syntactic, and relational information directly from raw unstructured input, eliminating brittle TF-IDF-style pipelines and enabling richer token-level representations.

Cross-modal and cross-source data fusion: LLMs can process heterogeneous inputs-such as narratives, timestamps, medication lists, and structured fields-within a single attention mechanism, improving throughput and reducing the need for multiple complex preprocessing stages.

Parameter-efficient adaptation: Techniques such as LoRA and QLoRA allow rapid domain adaptation using as little as 0.1-1% of parameters, avoiding full-model retraining cycles that are typical of conventional ML workflows.

Long-range dependency modeling: Transformer attention captures relationships across hundreds or thousands of tokens, beyond the receptive-field limits of logistic regression, SVMs with bag-of-words features, and shallow neural networks.

Domain-adaptive prompt conditioning: LLMs can adjust their behavior based on structured prompts, chain-of-thought instructions, or domain-specific reasoning templates without altering model weights, enabling fast task adaptation with no retraining.

Robustness to missing or incomplete structured data: LLMs can compensate for absent features by inferring latent relationships from available context, whereas traditional ML models typically require explicit imputation logic and remain sensitive to missing data patterns.

Together, these capabilities represent concrete, non-conventional technical enhancements that materially improve data processing, inference quality, and system-level functionality beyond what traditional machine-learning architectures can provide.

### Accuracy and Performance Improvements Over Traditional Machine Learning Techniques

LLM-based architectures also deliver measurable accuracy and performance improvements over traditional machine-learning models across a wide variety of classification, extraction, and prediction tasks. Empirical studies show that transformer-based LLMs often achieve substantially higher accuracy, precision, recall, and F1 scores than classical baselines such as SVMs, logistic regression, convolutional neural networks (CNNs), long short-term memory networks (LSTMs), and rule-based NLP systems.

Across multiple domains including enhancement-approval prediction, sentiment and sarcasm detection, biomedical information extraction, and clinical risk prediction LLMs have been shown to provide double-digit gains in accuracy and significant improvements in minority-class recall, addressing failure modes in which traditional models underperform. When combined with parameter-efficient fine-tuning or structured, multi-layer prompting, LLMs can outperform or closely match state-of-the-art classical models even under challenging evaluation regimes, such as chronologically split test sets or cost-sensitive settings that heavily penalize critical errors like false negatives.

LLMs also demonstrate markedly higher robustness to noisy, ambiguous, or incomplete inputs and can effectively leverage contextual signals (e.g., metadata, rich narratives, and long-form text) that traditional models cannot easily encode. These improvements show that LLM-based architectures not only enhance prediction quality but also materially elevate overall system performance beyond what is achievable with conventional machine-learning techniques.

For example, enhanced transformer models such as HS-BERT achieve approximately 96-98% accuracy on e-commerce comment classification tasks, substantially outperforming SVM, CNN, and Naive Bayes baselines that typically achieve around 70-92% accuracy. In software-engineering workflows, LoRA-tuned LLMs such as Llama 3.1 can improve enhancement-approval prediction accuracy from roughly 64% to 79%, while increasing minority-class (“approved”) recall by more than 12 percentage points relative to LSTM- and SVM-based systems.

In clinical and biomedical settings, LLMs such as GPT-4o, PaLM-2, and Llama-2 demonstrate superior extraction accuracy for complex tasks like identifying immunosuppressed patients from free-text notes or extracting patient-reported outcomes, consistently outperforming rule-based NLP pipelines and structured-data ML models. In cost-sensitive medical prediction tasks, LLMs equipped with structured prompts achieve higher recall and lower critical false-negative rates than traditional classifiers, improving performance under real-world risk-weighted metrics.

Even in domains where classical models already perform strongly such as fake-news detection BERT-style language models provide incremental but statistically significant gains, improving accuracy from about 99.81% to 99.98% and F1 scores from approximately 0.9980 to 0.9998.

Collectively, these results demonstrate that LLMs consistently deliver measurable, statistically significant improvements in accuracy, recall, F1 score, robustness, and minority-class performance compared to traditional machine-learning techniques, thereby materially improving the overall functioning and effectiveness of AI-driven computing systems.

1. A computer-implemented method, the method comprising:
receiving an input query;
generating, by a machine learning model and based on the input query, a compressed reasoning trace comprising a sequence of symbolic reasoning steps, wherein the compressed reasoning trace has a compressed token count that is less than a natural language token count of a natural language reasoning trace produced by a natural language model using natural language reasoning and produced in response to the input query, wherein the lesser token count reduces an inference-time computational load on the machine learning model, individual symbolic reasoning steps in the sequence of symbolic reasoning steps comprising:
an operator token selected from a set of predefined reasoning operators, and
a symbolic expression token associated with the operator token;
identifying, in the compressed reasoning trace, an answer operator indicating that the compressed reasoning trace has concluded; and
extracting a final answer from an answer expression that corresponds to the answer operator in the compressed reasoning trace.

2. The method of claim 1, wherein the symbolic expression token is associated with the operator token, the symbolic expression token encoding a logical operation applicable to:
a variable,
a numerical value,
a function symbol, or
any combination thereof.

3. The method of claim 1, wherein the machine learning model has an inference-time latency that is at least 25% less than the inference-time latency of the natural language model.

4. The method of claim 1, wherein the compressed token count of the compressed reasoning trace is between 1% and 15% of the natural language token count of the natural language reasoning trace.

5. The method of claim 1, wherein the compressed reasoning trace has at least 90% of an accuracy of the natural language reasoning trace generated by the natural language model in response to the input query.

6. A server comprising:
one or more processors; and
one or more non-transitory computer readable media storing instructions executable by the one or more processors to perform operations comprising:
receiving an input query;
generating, by a machine learning model and based on the input query, a compressed reasoning trace comprising a sequence of symbolic reasoning steps, wherein the compressed reasoning trace has a compressed token count that is less than a natural language token count of a natural language reasoning trace produced by a natural language model using natural language reasoning and produced in response to the input query, wherein the lesser token count reduces an inference-time computational load on the machine learning model, individual symbolic reasoning steps in the sequence of symbolic reasoning steps comprising:
an operator token selected from a set of predefined reasoning operators, and
a symbolic expression token associated with the operator token;
identifying, in the compressed reasoning trace, an answer operator indicating that the compressed reasoning trace has concluded; and
extracting a final answer from an answer expression that corresponds to the answer operator in the compressed reasoning trace.

7. The server of claim 6, wherein generating, by the machine learning model and based on the input query, the compressed reasoning trace comprises:
generating, by the machine learning model and based on:
the sequence of symbolic reasoning steps and
a complexity of the input query,
the compressed reasoning trace; and
wherein the complexity of the input query comprises:
a number of reasoning operations required to produce the final answer.

8. The server of claim 7, wherein the complexity of the input query is determined based at least in part on determining, in the input query:
a number of words,
a number of instructions,
a number of conditions; or
any combination thereof.

9. The server of claim 6, wherein the sequence of symbolic reasoning steps is ordered based on a time that individual reasoning steps, in the sequence of symbolic reasoning steps, are generated, wherein a second reasoning step generated at a later time than a first reasoning step is located after the first reasoning step in the sequence of symbolic reasoning steps.

10. The server of claim 6, wherein the machine learning model autoregressively generates the compressed reasoning trace by sequentially predicting individual tokens in the compressed reasoning trace conditioned on previously generated tokens.

11. The server of claim 6, wherein the set of predefined reasoning operators comprises:
a variable assignment operator,
an equation definition operator,
a case branching operator,
a solve operator,
a calculation operator,
a difference operator,
an answer operator; or
any combination thereof.

12. The server of claim 6, wherein individual symbolic reasoning steps comprise an ordered sequence that includes:
the operator token,
a delimiter character, and
the symbolic expression token.

13. The server of claim 6, wherein successive individual symbolic reasoning steps are concatenated using a step separator character to generate the compressed reasoning trace.

14. A non-transitory memory device to store instructions executable by one or more processors to perform operations comprising:
receiving an input query;
generating, by a machine learning model and based on the input query, a compressed reasoning trace comprising a sequence of symbolic reasoning steps, wherein the compressed reasoning trace has a compressed token count that is less than a natural language token count of a natural language reasoning trace produced by a natural language model using natural language reasoning and produced in response to the input query, wherein the lesser token count reduces an inference-time computational load on the machine learning model, individual symbolic reasoning steps in the sequence of symbolic reasoning steps comprising:
an operator token selected from a set of predefined reasoning operators, and
a symbolic expression token associated with the operator token;
identifying, in the compressed reasoning trace, an answer operator indicating that the compressed reasoning trace has concluded; and
extracting a final answer from an answer expression that corresponds to the answer operator in the compressed reasoning trace.

15. The non-transitory memory device of claim 14, wherein the compressed reasoning trace does not include natural language words.

16. The non-transitory memory device of claim 14, wherein the machine learning model is a large language model (LLM).

17. The non-transitory memory device of claim 14, wherein before receiving the input query, the machine learning model is trained by reinforcement learning comprising:
determining a set of training prompt-response pairs;
generating, by the machine learning model, a response to individual training prompts in a set of responses, wherein individual responses in the set of responses comprise a sequence of symbolic reasoning steps without natural language words;
performing a comparison of individual responses in the set of responses to a set of criteria;
computing a reward of individual responses based at least in part on the comparison; and
updating parameters of the machine learning model based on the rewards to bias the machine learning model toward generating correct responses while preserving accuracy of the correct responses.

18. The non-transitory memory device of claim 17, wherein the reward comprises a normalizing reward of the compressed reasoning trace of individual responses in the set of responses, based on a relative position of advantage of the compressed reasoning trace of individual responses within a range defined by a least advantaged and a most advantaged in the set of responses.

19. The non-transitory memory device of claim 17, wherein:
performing the comparison comprises performing the comparison of the final answer of individual responses in the set of responses to the set of criteria to create a subset of correct responses to individual training prompts;
computing the reward comprises computing the reward of a correct compressed reasoning trace of individual correct responses in the subset of correct responses based at least in part on a conditional reward function that applies different reward computations based on a composition of the subset of correct responses, the conditional reward function:
computing a length-preference reward of the correct compressed reasoning trace based on a relative position of a length of the correct compressed reasoning trace of individual correct responses within a range defined by a shortest length and a longest length in the subset of correct responses; and
updating parameters comprises updating parameters of the machine learning model based on the length-preference rewards to bias the machine learning model toward generating shorter correct responses while preserving accuracy of the correct responses.

20. The non-transitory memory device of claim 17, wherein before the machine learning model is trained by reinforcement learning, the machine learning model is trained by supervised fine-tuning to yield the machine learning model that is configured to generate the compressed reasoning trace in a symbolic reasoning language without translating from natural language, wherein the symbolic reasoning language is a native output format of the machine learning model.
